SEQUENCE LISTING (67 sequences)

SEQ ID NO: 1 - protein, 79 aa, Nicotiana benthamiana
PHDLKFLTEDESWILLEKKVFHKDKCPPELELSGKSIAKKCNGLPLAIVVIAGALIGKGKTSREWKQVDESVGEHLINK

SEQ ID NO: 2 - protein, 39 aa, Nicotiana benthamiana
PHDLKFLTEDESWILLEKKVFHKDKCPPELELSGKSIAK

SEQ ID NO: 3 - protein, 20 aa, Nicotiana benthamiana
HDLKFLTEDESWILLEKKVF

SEQ ID NO: 4 - protein, 79 aa, Nicotiana benthamiana; identical to SEQ ID NO: 1

SEQ ID NO: 5 - protein, 39 aa, Nicotiana benthamiana; identical to SEQ ID NO: 2

SEQ ID NO: 6 - protein, 20 aa, Nicotiana benthamiana
HDLKFLTEDESWILLEKKVF

SEQ ID NO: 7 - protein, 79 aa, Nicotiana benthamiana
PHDLKFLTENESWELLEKRVFHKEKCPFELELPGKSIAKKCRGLPLAIVVIAGALIGKGKTTREWELVADSVGEHLINR

SEQ ID NO: 8 - protein, 39 aa, Nicotiana benthamiana
PHDLKFLTENESWELLEKRVFHKEKCPFELELPGKSIAK

SEQ ID NO: 9 - protein, 20 aa, Nicotiana benthamiana
HDLKFLTENESWELLEKRVF

SEQ ID NO: 10 - protein, 79 aa, Nicotiana benthamiana
PHDLKFLTPKESFELLVKRVFGKKPCPKDLVGHGESIAGKCGGVPLAVVVIAGALRGRPNTSDWIRVERNVVQHLYTNS

SEQ ID NO: 11 - protein, 39 aa, Nicotiana benthamiana
PHDLKFLTPKESFELLVKRVFGKKPCPKDLVGHGESIAG

SEQ ID NO: 12 - protein, 20 aa, Nicotiana benthamiana
HDLKFLTPKESFELLVKRVF

SEQ ID NO: 13 - protein, 80 aa, Solanum tuberosum
PHDLKFLSEDESWILLEKKVFHKDKCPPELVVPSGKSIAKKCKGLPLAIVVIAGALIGKGKTPREWKQVDDSVSEHLINR

SEQ ID NO: 14 - protein, 40 aa, Solanum tuberosum
PHDLKFLSEDESWILLEKKVFHKDKCPPELVVPSGKSIAK

SEQ ID NO: 15 - protein, 20 aa, Solanum tuberosum
HDLKFLSEDESWILLEKKVF

SEQ ID NO: 16 - protein, 79 aa, Solanum tuberosum
PHDLKFLTEDESWELLEKKVFHKEKCPPELELPGKSIAEKCMGLPLAIVVIAGALIGKGKTTREWELVAASVREHLINR

SEQ ID NO: 17 - protein, 39 aa, Solanum tuberosum
PHDLKFLTEDESWELLEKKVFHKEKCPPELELPGKSIAE

SEQ ID NO: 18 - protein, 19 aa, Solanum tuberosum
DLKFLTEDESWELLEKKVF

SEQ ID NO: 19 - protein, 79 aa, Solanum tuberosum
PHVLKFLKTEESFELLVMRVFGKGSCPNELVVTGKKIAEKCGGVPLVVVVIAGALRGRSDKKDWERVDKNVVQHFGEHT

SEQ ID NO: 20 - protein, 39 aa, Solanum tuberosum
PHVLKFLKTEESFELLVMRVFGKGSCPNELVVTGKKIAE

SEQ ID NO: 21 - protein, 20 aa, Solanum tuberosum
HELKFLEKEESFELLVMRVF

SEQ ID NO: 22 - protein, 79 aa, Solanum lycopersicum
PHDLKFLTDEESWILLEKRAFHKAKCLPELETNGKSIARKCKGLPLAIVVIAGALIGKSKTIKEWEQVDQSVGEHFINR

SEQ ID NO: 23 - protein, 39 aa, Solanum lycopersicum
PHDLKFLTDEESWILLEKRAFHKAKCLPELETNGKSIAR

SEQ ID NO: 24 - protein, 19 aa, Solanum lycopersicum
HDLKFLTDEESWILLEKRA

SEQ ID NO: 25 - protein, 79 aa, Solanum lycopersicum
PHDLKFLTEDESWILLEKKVFHKDKCPPELVLSGKSIAKKCKGLPLAIVVIAGALIGKGKTPREWKQVDDSVSEHLINR

SEQ ID NO: 26 - protein, 39 aa, Solanum lycopersicum
PHDLKFLTEDESWILLEKKVFHKDKCPPELVLSGKSIAK

SEQ ID NO: 27 - protein, 20 aa, Solanum lycopersicum
HDLKFLTEDESWILLEKKVF

SEQ ID NO: 28 - protein, 79 aa, Solanum lycopersicum
PHDLKFLTKDESWELLEKKVFHKEKCPPELELPGISIAEKCMGLPLAIVVIAGALIGKGKTTREWELVAASVGEHLINR

SEQ ID NO: 29 - protein, 39 aa, Solanum lycopersicum
PHDLKFLTKDESWELLEKKVFHKEKCPPELELPGISIAE

SEQ ID NO: 30 - protein, 20 aa, Solanum lycopersicum
HDLKFLTKDESWELLEKKVF

SEQ ID NO: 31 - DNA, 2661 bp, Nicotiana benthamiana
  source 1..2661: mol_type = genomic DNA
atggcgaacgttgcggtggagtttctggtgcagaacttgatgcagctgctaagagacaatgcggagctgattgttggagttaaggattcggctgagagtctgcttcaagatctcaatgatttcaacgcttttctcaagcaaactgctaagtcccgcactgagaacgatgtccacaaagaattggtgaagaaaattaagactgtggtcaactctgctgaagatgccattgataagtttgtgattgaggctaagcttcacaaggacaaaggggttggcagatttgtggatgttaagcattataaaagagtgtatgatgtagcaggggagattaaaactattagagacaaagtcaaagaaattcgtctcaataatgcacttgaccttcaggcccttcaagatgaagatcaatctgccaaaggtgtccaagaaagaaagcctccggtggtagaggaagatgatgtggttggatttgaagaggaagcagataaagtaatcaaccgtcttctgggaggatcgagtggactagaagttgttccagttgttggaatgcctggtctcggcaaaacgacactggcaaataagatttacaagcatcctgacatcgggtaccagttttttactcgcatttgggtttatgtttctcaatcatacaggagaagagaattatttctcaacatcatcagcaaattcactcgcaataccaaacaataccacgatatgtgtgaggaggatttagctgatgaaatagaagattttttgggcaagggagggaaatacttgattgtcttggatgatgtatggtctcctgacgcttgggaacgtatcagaatagctttcccaaacaacaacaaatccaatagaatattattgaccacccgagatagcaaagttgctaagcaatgcaagcagtgcattggtatacctcatgatttaaaatttctgactgaagatgaaagttggattttactggagaagaaagttttccataaagataaatgtcctcctgaattggaactatctggaaagagcatagccaaaaaatgtaatggactaccccttgcaattgttgttattgcaggagcactaattgggaaaggtaagacatcaagagagtggaaacaagtggatgagagtgtgggtgaacacctcataaataaagaccagcctgagaattgtaacaaattggtgcaactgagttatgatcgcttgtcttacgacttgaaagcatgttttttgtattgtggtgcatttcctggaggctttgagatccctgcttggaagctaatccgtttgtggatcgcagaagggttcatacagtataaaggccacttatctcttgagtgtaaagcagaggataacttgaatgatctaattaacaggaatctagtgatggtaatgcaaagaacatctgatggtcaaatcaaaacatgtcgtcttcatgacatgttgcacgagttttgcagacaagaggctatgaaggaagaaaatcttttccaagaaataaaactaggtgctgagcaatatttccctggaaaacgggaactagccacctaccgtcgcttatgcattcattcctcagttttggaatttatctctacaaagccctcaggtgaacatgtcaggtcattcttatctttttctttaaaaaagattgagatgccatctgtcgacatcccaaccataccaaaaggctttccgttgctgagggtttttgatgtcgagtccatcaacttcagtcgcttttccaaggagtttttccagttatatcatttgaggtatattgctttctcatctgacacaatcaagatcattcctaaacacattggagaactgtggaacatccaaacactcataattaacacgcaacaacgctctcttgatatccaagcaaacatatggaatatggaacgactaaggcatctgcacactaactcttctgctaaattgcctgttcctgtgaccccaagaagtagtaaagttcctttggtaaatcaaagcctgcaaactctctccaccattgctcccgaaagctgcacagaagaagtgtttgcaaggactccaaacctgaaaaaactgggtattcgtgggaaaatagctgtgcttcttgaacctaataagtcattgttaaaaaatgtgaagaaactagaatcccttgaaaacttgaagctgataaatgatagtagtcaaacaggaaaagggttacgccttcctccctcatatatatttcccacgaagttgaggaagctatctttagtagatacctggctggagtggaatgatatgtctatattgggacagatggaacaccttgaagtcctgaagctgaaagaaaatgggtttatgggagaatgctgggagtcagttggaggtttttgttccctactggtgctgtggattgaaaggacagatttagtttcttggaaagcatcagctgatcactttccaagacttaagcatcttgttctcatctgctgcgataagcttaaggaaatccccattggcctggctgatatacgcagcttccaagtgatggagttgcaaaactcaaccaaaacagcagcaatatctgcacggggaatacgagacaaaaaagacaagcaaactcaagaagggactaacaataatgggttcaagctctctatattccctcccgatctctaa

SEQ ID NO: 32 - protein, 886 aa, Nicotiana benthamiana
MANVAVEFLVQNLMQLLRDNAELIVGVKDSAESLLQDLNDFNAFLKQTAKSRTENDVHKELVKKIKTVVNSAEDAIDKFVIEAKLHKDKGVGRFVDVKHYKRVYDVAGEIKTIRDKVKEIRLNNALDLQALQDEDQSAKGVQERKPPVVEEDDVVGFEEEADKVINRLLGGSSGLEVVPVVGMPGLGKTTLANKIYKHPDIGYQFFTRIWVYVSQSYRRRELFLNIISKFTRNTKQYHDMCEEDLADEIEDFLGKGGKYLIVLDDVWSPDAWERIRIAFPNNNKSNRILLTTRDSKVAKQCKQCIGIPHDLKFLTEDESWILLEKKVFHKDKCPPELELSGKSIAKKCNGLPLAIVVIAGALIGKGKTSREWKQVDESVGEHLINKDQPENCNKLVQLSYDRLSYDLKACFLYCGAFPGGFEIPAWKLIRLWIAEGFIQYKGHLSLECKAEDNLNDLINRNLVMVMQRTSDGQIKTCRLHDMLHEFCRQEAMKEENLFQEIKLGAEQYFPGKRELATYRRLCIHSSVLEFISTKPSGEHVRSFLSFSLKKIEMPSVDIPTIPKGFPLLRVFDVESINFSRFSKEFFQLYHLRYIAFSSDTIKIIPKHIGELWNIQTLIINTQQRSLDIQANIWNMERLRHLHTNSSAKLPVPVTPRSSKVPLVNQSLQTLSTIAPESCTEEVFARTPNLKKLGIRGKIAVLLEPNKSLLKNVKKLESLENLKLINDSSQTGKGLRLPPSYIFPTKLRKLSLVDTWLEWNDMSILGQMEHLEVLKLKENGFMGECWESVGGFCSLLVLWIERTDLVSWKASADHFPRLKHLVLICCDKLKEIPIGLADIRSFQVMELQNSTKTAAISARGIRDKKDKQTQEGTNNNGFKLSIFPPDL

SEQ ID NO: 33 - DNA, 2661 bp, Nicotiana benthamiana
  source 1..2661: mol_type = genomic DNA
atggcgaacgttgcggtggaatttttggtgcagaacttgatgcagctgctgagggacaatgcggagctgattattggggttaaggattcggctgagagcctgcttcaagatctcaatgatttcaacgcttttctcaagcaagctgccaagtcccgcattgagaacgatgtccacaaagaattggtgaagaaaattaagacaatggtcaactctgctgaagatgccattgataagtttgtaattgaggctaagcttcacaaggacaaaggggttggcagatttgtagatgttaagcattataaaagagtgtatgatgtagcagccgagatcaaaggtatcagagaaaaagtgaaagaaatccgtcagaataatgcccttgaccttcaagcccttcaagatgaagatcaatctgccaaaggtgtcgaagaaagaaagcctccagtggtagaggaagatgatgtggttggatttgaagaggaagcagataaagtaatcaaccgtcttctgggaggattgagtggactagaagttgttccagttgttggaatgcctggtctcggcaaaacgacgctagcaaataagatttacaagcatcctgacatcgggtaccagttttttactcgcatttgggtttatgtttctcaatcatacaggagaagagaattatttctcaacatcatcagcaaattcactcgaaataccaaacaataccatgatatgtgtgaggaggatttagctgatgaaatacaagattttttgggcaaaggaggaaaatacttgattgtcttggatgatgtatggtctcctgacgcttgggaacacatcaaaatagctttcccgaacaacaacaaatccaatagaatattattgaccactcgagatagcaaagttgctaagcaatgcaagcagtgcattggtatacctcatgatttaaaatttctgactgaagatgaaagttggattttactggagaagaaagttttccataaagataaatgtcctcctgaattggaactatctggaaagagcatagccaaaaaatgtaatggactaccccttgcgattgttgttattgcaggagcactaattgggaaaggtaagacatcaagagagtggaaacaagtggatgagagtgtgggtgaacacctcataaataaagaccagcctgagaattgtaacaaattggtgcaactgagttatgatcgcttgtcttatgacttgaaagcatgttttttatattgtggtgcatttcccggaggctttgagatccctgcttggaagttaatccgtttgtggatcgcagaagggttcatacagtataaaggccacttatctcttgagtgtaaagcagaggataacttgaatgatctcatcaacaggaatctagtgatggtaatgcaaaggacatctgatgggcaaatcaaaacatgtcgtcttcatgacatgttgcacgagttttgcagacaagaggcgatgaaggaagaaaatcttttccaagaaataaaattaggtgctgagcaatatttcccaggaaaacgggaactagccacctaccgtcgcttatgcattcattcctcagttttggaatttatctctacaaagccctcaggtgaacatgtcaggtcattcttatctttttctttaaaaaaggtcgagatgccatctgtcgacatcccaaccataccaaaaggcttcccgttgctgagggtttttgatgtcgagtccatcaacttcagtcgcttttccaaggagtttttccagttatatcatttgaggtatattgctttctcatctgacacaatcaagatcattcctaaacacattggagaactgtggaacatccaaacactcataattaacacgcaacaacactctcttgatatccaagcaaacatatggaatatggcacgactaaggcatctgcacactaactcttctgctaaattgcctgttcctgtgaccccaagaagtagtaaagttcctttggtaaatcaaagcctgcaaactctctccaccattgctcccgaaagctgcacagaagaagtgtttgcaaggactccaaacctgaaaaagttgggtattcgtgggaaagttgctgtgcttcttgaacctaataagtcattgttaaaaaatgtgaagaaactagaatcccttgaaaacttgaagctgataaatgatagtagtcaaacaggaaaagggttacgccttccaccctcatatatatttcccacgaagttgaggaagctatctttagtagatacctggctggagtggaacgatatgtctatattggggcagatggaacaccttgaagttctgaagctgaaagaaaatgggtttatgggagaatgctgggagtcagttggaggtttttgttccctattggtgttgtggattgaaaggacagacttagtttcttggaaagcatcagctgatcactttccaagacttaagcatcttgttctcatctgctgcgataagattaaggaaatccccattggcctggctgatatacacagcttccaagtgatggagttgcaaaactccaccaaaacagcagcaatatctgcacgggaaatacgagacaaaaaagacaagcaaactcaagaggggactaacaacaatgggttcaagctctctatattccctcccgatctctga

SEQ ID NO: 34 - protein, 886 aa, Nicotiana benthamiana
MANVAVEFLVQNLMQLLRDNAELIIGVKDSAESLLQDLNDFNAFLKQAAKSRIENDVHKELVKKIKTMVNSAEDAIDKFVIEAKLHKDKGVGRFVDVKHYKRVYDVAAEIKGIREKVKEIRQNNALDLQALQDEDQSAKGVEERKPPVVEEDDVVGFEEEADKVINRLLGGLSGLEVVPVVGMPGLGKTTLANKIYKHPDIGYQFFTRIWVYVSQSYRRRELFLNIISKFTRNTKQYHDMCEEDLADEIQDFLGKGGKYLIVLDDVWSPDAWEHIKIAFPNNNKSNRILLTTRDSKVAKQCKQCIGIPHDLKFLTEDESWILLEKKVFHKDKCPPELELSGKSIAKKCNGLPLAIVVIAGALIGKGKTSREWKQVDESVGEHLINKDQPENCNKLVQLSYDRLSYDLKACFLYCGAFPGGFEIPAWKLIRLWIAEGFIQYKGHLSLECKAEDNLNDLINRNLVMVMQRTSDGQIKTCRLHDMLHEFCRQEAMKEENLFQEIKLGAEQYFPGKRELATYRRLCIHSSVLEFISTKPSGEHVRSFLSFSLKKVEMPSVDIPTIPKGFPLLRVFDVESINFSRFSKEFFQLYHLRYIAFSSDTIKIIPKHIGELWNIQTLIINTQQHSLDIQANIWNMARLRHLHTNSSAKLPVPVTPRSSKVPLVNQSLQTLSTIAPESCTEEVFARTPNLKKLGIRGKVAVLLEPNKSLLKNVKKLESLENLKLINDSSQTGKGLRLPPSYIFPTKLRKLSLVDTWLEWNDMSILGQMEHLEVLKLKENGFMGECWESVGGFCSLLVLWIERTDLVSWKASADHFPRLKHLVLICCDKIKEIPIGLADIHSFQVMELQNSTKTAAISAREIRDKKDKQTQEGTNNNGFKLSIFPPDL

SEQ ID NO: 35 - DNA, 2667 bp, Nicotiana benthamiana
  source 1..2667: mol_type = genomic DNA
atggcagatgtagcagcagatgtagcagtaaaattcctagtagaaaacttgatgcaattattaatcgacaacgctgacttgattattggtataaagggtgaagttgaaaatttactacaagatctgaatgatttcaatgcttttctcaaacaagcagctaaatcaaggagagacaatgaagtcttgaaatcactagtaaagaagataaggaaagtggtaaatgatgctgaagattcaattgataagtttgtgattgaagctaaaagacatgacgataaaaataaatttgctcagtggtttcatcttactcatgttgctagagcaaaaggggttgctgatgagattaaaactataagggaaagggtgaaggaaattcgccaaaatgatgcttatggacttcaagctataacttcttatgataatttcaaccaaggtgctcaggagaggaaggtccctgtagttgaggaagacgacgtggtaggctttgatgacgaagcgaaaactgtaattgatcgcctcattggaggatcagattatgttgtgccggtcgttggtatgcctggtcttggaaaaacaactttggcatataagattttcaaggattccacagttgagtatgagtttttcaaccgcatatgggtatatgtctctcaatcattcaacagaagggaaatatttctcaacatcatcagcaaattcactcgaaacaccaaacaataccatgatacaccagaggaggaattagcaaatgaaataaaggagctgcttgggaagggtgggaaatatcttgttgttttagatgatgtgtggacaagagaagcttgggatcgcattaaaattgctttccccaataataataaacggaatagagtcttgatgactactagacaaaacaatgtggctaagtcttgcaacgataaacctcatgatctaaagtttttgactgaaaatgaaagttgggagctacttgagaagagagtttttcacaaggaaaaatgtccatttgagttagaattacctggaaaaagtatagccaaaaaatgtaggggcttaccacttgcaatagttgtcattgctggagctttaataggcaaaggaaagacgacaagggagtgggagctggtggctgacagtgtgggtgagcacctcataaatagagatccagagaactgcaagaaattggtgcagatgagttatgaccgtttgccttatgatttaaaggcgtgcttcttatattgtggggcttttcctggcggttctgaaatttcagctcagaagttgatttgtttatggattgcggaaggattcatacaataccaaggaccattgactctagaggatatagcagaggaccacttgaatgatcttgtcaataggaacttagtgatggtaatgaaaaggagttctagcggtcaaatcaaaacatgtcgtgttcatgacatgttgcatgagttctgcagacatgaagctatgatggaggaaaatctattccaagaaatcaaacgagggcaagaacattcttttccagagaaacaagagttagcctcttatcgtcgcttgtgcattcattcgtctgtttctgaattcttgtcaacaaaaccctttgctgaacatgttaggtcgttcttatgctttgcctcgaaaaaatttgagatgcctttaggtgaaataccagccatccccagagcctttcccttgcttagggtactcgatgctgaatcaatcaagttcagtcgtttttctagagagttcttcaaattattccatttgaggtacattgctttctcaactgactcgattatgaccattcctacaaacattgggaatctttggaacgtacaaacacttataattgagacacaacagggtactcttgacattaaagcagacatttggaatatgacaagattaaggcatgtgtgcataaacgcctctgctacattgccttcccctaagcgccccaagagtagcaaggacaacttggtgaatcgttgcctacaaacactttctaccatagcacctgaatgttgcacggcagaagtttttactagaactcctaatctcaagaaattaggtgttcgtggaaaaatagatgcacttctagaaaccagtaaggacgggtccagttctggtttgttcagcaacataggaaagttagattgcctcgaaaagctgaagttggtaaatgatactcgtcaaagtagaaaacagctacaccttcctccagcatatatttttcctcagaagctaaaaaaactcactctgatagatacgtggttcgaatggaaagatatgtctatattaggtctattagagtaccttgaagtgctgaagttgaaggaaaatgcgtttaggggacagtcgtgggaaccagaggatagtggttttcctcgtctgcaggtcctatggattgaaaggacagatctatcttcttggaaggcctcatctggaaactttccacgactaaaatgccttgttcttatagcatgtgataatcttaaggaactaccagctgaactggctgatgtggaaaacctccagttaatggaactccagagtaccagtgtgtctgctgctaaatctgcacgagcaatactaaagaagaaacaacaaaaagttggcagcggattcaagctttctgtatttcctccagatctcggattgtaa

SEQ ID NO: 36 - protein, 888 aa, Nicotiana benthamiana
MADVAADVAVKFLVENLMQLLIDNADLIIGIKGEVENLLQDLNDFNAFLKQAAKSRRDNEVLKSLVKKIRKVVNDAEDSIDKFVIEAKRHDDKNKFAQWFHLTHVARAKGVADEIKTIRERVKEIRQNDAYGLQAITSYDNFNQGAQERKVPVVEEDDVVGFDDEAKTVIDRLIGGSDYVVPVVGMPGLGKTTLAYKIFKDSTVEYEFFNRIWVYVSQSFNRREIFLNIISKFTRNTKQYHDTPEEELANEIKELLGKGGKYLVVLDDVWTREAWDRIKIAFPNNNKRNRVLMTTRQNNVAKSCNDKPHDLKFLTENESWELLEKRVFHKEKCPFELELPGKSIAKKCRGLPLAIVVIAGALIGKGKTTREWELVADSVGEHLINRDPENCKKLVQMSYDRLPYDLKACFLYCGAFPGGSEISAQKLICLWIAEGFIQYQGPLTLEDIAEDHLNDLVNRNLVMVMKRSSSGQIKTCRVHDMLHEFCRHEAMMEENLFQEIKRGQEHSFPEKQELASYRRLCIHSSVSEFLSTKPFAEHVRSFLCFASKKFEMPLGEIPAIPRAFPLLRVLDAESIKFSRFSREFFKLFHLRYIAFSTDSIMTIPTNIGNLWNVQTLIIETQQGTLDIKADIWNMTRLRHVCINASATLPSPKRPKSSKDNLVNRCLQTLSTIAPECCTAEVFTRTPNLKKLGVRGKIDALLETSKDGSSSGLFSNIGKLDCLEKLKLVNDTRQSRKQLHLPPAYIFPQKLKKLTLIDTWFEWKDMSILGLLEYLEVLKLKENAFRGQSWEPEDSGFPRLQVLWIERTDLSSWKASSGNFPRLKCLVLIACDNLKELPAELADVENLQLMELQSTSVSAAKSARAILKKKQQKVGSGFKLSVFPPDLGL

SEQ ID NO: 37 - protein, 886 aa, Nicotiana benthamiana
MANVAVEFLVQNLMQLLRDNAELIVGVKDSAESLLQDLNDFNAFLKQTAKSRTENDVHKELVKKIKTVVNSAEDAIDKFVIEAKLHKDKGVGRFVDVKHYKRVYDVAGEIKTIRDKVKEIRLNNALDLQALQDEDQSAKGVQERKPPVVEEDDVVGFEEEADKVINRLLGGSSGLEVVPVVGMPGLGKTTLANKIYKHPDIGYQFFTRIWVYVSQSYRRRELFLNIISKFTRNTKQYHDMCEEDLADEIEDFLGKGGKYLIVLDDVWSPDAWERIRIAFPNNNKSNRILLTTRDSKVAKQCKQCIGIPHDLKFLTEKESWILLEKKVFHKDKCPPELELSGKSIAKKCNGLPLAIVVIAGALIGKGKTSREWKQVDESVGEHLINKDQPENCNKLVQLSYDRLSYDLKACFLYCGAFPGGFEIPAWKLIRLWIAEGFIQYKGHLSLECKAEDNLNDLINRNLVMVMQRTSDGQIKTCRLHDMLHEFCRQEAMKEENLFQEIKLGAEQYFPGKRELATYRRLCIHSSVLEFISTKPSGEHVRSFLSFSLKKIEMPSVDIPTIPKGFPLLRVFDVESINFSRFSKEFFQLYHLRYIAFSSDTIKIIPKHIGELWNIQTLIINTQQRSLDIQANIWNMERLRHLHTNSSAKLPVPVTPRSSKVPLVNQSLQTLSTIAPESCTEEVFARTPNLKKLGIRGKIAVLLEPNKSLLKNVKKLESLENLKLINDSSQTGKGLRLPPSYIFPTKLRKLSLVDTWLEWNDMSILGQMEHLEVLKLKENGFMGECWESVGGFCSLLVLWIERTDLVSWKASADHFPRLKHLVLICCDKLKEIPIGLADIRSFQVMELQNSTKTAAISARGIRDKKDKQTQEGTNNNGFKLSIFPPDL

SEQ ID NO: 38 - protein, 886 aa, Nicotiana benthamiana
MANVAVEFLVQNLMQLLRDNAELIIGVKDSAESLLQDLNDFNAFLKQAAKSRIENDVHKELVKKIKTMVNSAEDAIDKFVIEAKLHKDKGVGRFVDVKHYKRVYDVAAEIKGIREKVKEIRQNNALDLQALQDEDQSAKGVEERKPPVVEEDDVVGFEEEADKVINRLLGGLSGLEVVPVVGMPGLGKTTLANKIYKHPDIGYQFFTRIWVYVSQSYRRRELFLNIISKFTRNTKQYHDMCEEDLADEIQDFLGKGGKYLIVLDDVWSPDAWEHIKIAFPNNNKSNRILLTTRDSKVAKQCKQCIGIPHDLKFLTEKESWILLEKKVFHKDKCPPELELSGKSIAKKCNGLPLAIVVIAGALIGKGKTSREWKQVDESVGEHLINKDQPENCNKLVQLSYDRLSYDLKACFLYCGAFPGGFEIPAWKLIRLWIAEGFIQYKGHLSLECKAEDNLNDLINRNLVMVMQRTSDGQIKTCRLHDMLHEFCRQEAMKEENLFQEIKLGAEQYFPGKRELATYRRLCIHSSVLEFISTKPSGEHVRSFLSFSLKKVEMPSVDIPTIPKGFPLLRVFDVESINFSRFSKEFFQLYHLRYIAFSSDTIKIIPKHIGELWNIQTLIINTQQHSLDIQANIWNMARLRHLHTNSSAKLPVPVTPRSSKVPLVNQSLQTLSTIAPESCTEEVFARTPNLKKLGIRGKVAVLLEPNKSLLKNVKKLESLENLKLINDSSQTGKGLRLPPSYIFPTKLRKLSLVDTWLEWNDMSILGQMEHLEVLKLKENGFMGECWESVGGFCSLLVLWIERTDLVSWKASADHFPRLKHLVLICCDKIKEIPIGLADIHSFQVMELQNSTKTAAISAREIRDKKDKQTQEGTNNNGFKLSIFPPDL

SEQ ID NO: 39 - protein, 888 aa, Nicotiana benthamiana
MADVAADVAVKFLVENLMQLLIDNADLIIGIKGEVENLLQDLNDFNAFLKQAAKSRRDNEVLKSLVKKIRKVVNDAEDSIDKFVIEAKRHDDKNKFAQWFHLTHVARAKGVADEIKTIRERVKEIRQNDAYGLQAITSYDNFNQGAQERKVPVVEEDDVVGFDDEAKTVIDRLIGGSDYVVPVVGMPGLGKTTLAYKIFKDSTVEYEFFNRIWVYVSQSFNRREIFLNIISKFTRNTKQYHDTPEEELANEIKELLGKGGKYLVVLDDVWTREAWDRIKIAFPNNNKRNRVLMTTRQNNVAKSCNDKPHDLKFLTEKESWELLEKRVFHKEKCPFELELPGKSIAKKCRGLPLAIVVIAGALIGKGKTTREWELVADSVGEHLINRDPENCKKLVQMSYDRLPYDLKACFLYCGAFPGGSEISAQKLICLWIAEGFIQYQGPLTLEDIAEDHLNDLVNRNLVMVMKRSSSGQIKTCRVHDMLHEFCRHEAMMEENLFQEIKRGQEHSFPEKQELASYRRLCIHSSVSEFLSTKPFAEHVRSFLCFASKKFEMPLGEIPAIPRAFPLLRVLDAESIKFSRFSREFFKLFHLRYIAFSTDSIMTIPTNIGNLWNVQTLIIETQQGTLDIKADIWNMTRLRHVCINASATLPSPKRPKSSKDNLVNRCLQTLSTIAPECCTAEVFTRTPNLKKLGVRGKIDALLETSKDGSSSGLFSNIGKLDCLEKLKLVNDTRQSRKQLHLPPAYIFPQKLKKLTLIDTWFEWKDMSILGLLEYLEVLKLKENAFRGQSWEPEDSGFPRLQVLWIERTDLSSWKASSGNFPRLKCLVLIACDNLKELPAELADVENLQLMELQSTSVSAAKSARAILKKKQQKVGSGFKLSVFPPDLGL

SEQ ID NO: 40 - DNA, 2661 bp, Solanum tuberosum
  source 1..2661: mol_type = genomic DNA
atggcgaacgtagcagtagaatttctcgttgagaacttgatgcagctgctgcgggacaacgcagagctaattagtggagttaaagaggctgctgagagtctacttcaagatctaaatgatttcaacgcctttcttaagcaagctgccaagtgccacatcaacgagaacgaagttctgagagaactcgtcaagaaaatcagaacagtggttaactctgctgaagatgctattgataaatttgtgattgaagctaagctacacaaggataagggtatgaccagagtattagaccttccccattataaaagagtcagggaggtagctggtgagatcaaagctatacgaaacaaagtcaaagagatcagacagaatgatgccattggacttcaggcccttcaagatgatgattcatctgccagaggtttcgaagaaagaaagcctccagtggtagaggaagatgatgtggttggatttgacgaagaagcagatattgtaatcaaacgccttcttggagaatcaaatcgtctagaagttgttccagttgttggtatgcctggtctcggcaaaacgaccctagcaaataaaatatacaagcatcctaaaatcgggtatgaattttttactcgtatttgggtttatgtatctcaatcatacaggagaagagaattatttctcaacatcatcagcaaattcactcgaaatacgaaacaatatcatgggatgtgtgaggaggatttggctgatgaaatacaagaattcttgggaaagggaggaaaatacttggttgtcttggatgatgtatggtcggatgaagcttgggaacgtatcaagatagctttcccaaataacaacaaaccgaatcgagtattgttgaccactcgagattccaaagtggctaaacaatgcactcccatacctcatgatttaaaatttctgagtgaagatgaaagttggatattactggagaagaaggtttttcacaaggataaatgtcctcctgaattggtggtaccatctgggaagagcatagcaaaaaaatgtaagggactaccccttgcgattgttgttatcgcaggagcactaatagggaaaggcaagacaccaagagagtggaaacaagtggatgatagtgtgagtgaacacctcataaatagagaccaccctgagaattgtaacaaactggtgcaaatgagttatgatcgtttaccttacgacttgaaagcgtgctttttatattgtagtgcatttcccggaggctttcaaatccctgcttggaagttaatccgtttgtggattgcagaaggtttcatacagtataaaggccacttatctcttgagtgtaaaggagaggacaacttgaatgatctcatcaacaggaatctagtgatggtaatggaaagaacgtctgatggtcaaatcaaaacatgtcgtcttcatgacatgttgcacgagttttgcaggcaagaagctatgaaggaagaaaatcttttccaagaaataaagctaggatctgagcaatattttcctggaaaacgagaactatccacgtaccgtcgcttatgcattcattcctcagttttggattttatctctacgaaaccttcagctgaacatgtgaggtcattcttatctttttcttcaaaaaagattgagatgccatctgctgacatcccaaccataccaaaaggctttccgttgctaagagttttggatgtagaatccatcaacttcagtcgcttttccaaggagttttaccagttatatcatttgaggtatgttgctttttcatctgactcaatcaagattcttcctaaactcatgggagaactttggaacatccaaaccatcataattaacacgcaacaacgcactcttgatattcaagcaaatatatggaatatggaacgactaaggcatctgcacactaactcttctgctaaattgcctgtccctgttgctccaaaaaatagtaaagttacattggtaaatcaaagcctgcaaactctctccacaatagctcccgaaagctgcactgaagaagtgtttgcaaggactccaaacctgaaaaagctgggcatccgtgggaaaatagctgtgcttcttgataataagtctgctgtgtcgttaaaaaatgtgaagaggctagaataccttgaaaacttgaagctgataaatgatagtagcattcaaacaggaaagttacgccttccacctgcatacatatttccaacaaagttgaggaagctgactttattagacacctggttggagtggaaagatatgtctatattgggtcagttggaacaccttgaagtcctgaagatgaaagaaaatgggtttacgggagagtcctgggagtctactggtggtttttgttccctactggtgttgtggattgaaaggacaaacttagttacttggaaagcatcagctgatgactttccaagacttaagcatcttgttctcatctgctgtgattaccttaaggaagtccccatcgccctagctgatatacgcagtttccaagtcatgatgttgcaaaattccaccaaaacagcagcaatatctgcacgtcaaatacaagccaaaaaagacaatcaaactcaacaagggactaaaaacattgccttcaagctttccatattccctcctgatctctga

SEQ ID NO: 41 - protein, 886 aa, Solanum tuberosum
MANVAVEFLVENLMQLLRDNAELISGVKEAAESLLQDLNDFNAFLKQAAKCHINENEVLRELVKKIRTVVNSAEDAIDKFVIEAKLHKDKGMTRVLDLPHYKRVREVAGEIKAIRNKVKEIRQNDAIGLQALQDDDSSARGFEERKPPVVEEDDVVGFDEEADIVIKRLLGESNRLEVVPVVGMPGLGKTTLANKIYKHPKIGYEFFTRIWVYVSQSYRRRELFLNIISKFTRNTKQYHGMCEEDLADEIQEFLGKGGKYLVVLDDVWSDEAWERIKIAFPNNNKPNRVLLTTRDSKVAKQCTPIPHDLKFLSEDESWILLEKKVFHKDKCPPELVVPSGKSIAKKCKGLPLAIVVIAGALIGKGKTPREWKQVDDSVSEHLINRDHPENCNKLVQMSYDRLPYDLKACFLYCSAFPGGFQIPAWKLIRLWIAEGFIQYKGHLSLECKGEDNLNDLINRNLVMVMERTSDGQIKTCRLHDMLHEFCRQEAMKEENLFQEIKLGSEQYFPGKRELSTYRRLCIHSSVLDFISTKPSAEHVRSFLSFSSKKIEMPSADIPTIPKGFPLLRVLDVESINFSRFSKEFYQLYHLRYVAFSSDSIKILPKLMGELWNIQTIIINTQQRTLDIQANIWNMERLRHLHTNSSAKLPVPVAPKNSKVTLVNQSLQTLSTIAPESCTEEVFARTPNLKKLGIRGKIAVLLDNKSAVSLKNVKRLEYLENLKLINDSSIQTGKLRLPPAYIFPTKLRKLTLLDTWLEWKDMSILGQLEHLEVLKMKENGFTGESWESTGGFCSLLVLWIERTNLVTWKASADDFPRLKHLVLICCDYLKEVPIALADIRSFQVMMLQNSTKTAAISARQIQAKKDNQTQQGTKNIAFKLSIFPPDL

SEQ ID NO: 42 - DNA, 2676 bp, Solanum tuberosum
  source 1..2676: mol_type = genomic DNA
atggcggatgtagcagtaaagtttttagtagaaaatttgatgcaattactaatcgacaacgccgatttgattctcggaatcaaaggcgaagttgaaaatctactccgagatctcaatgacttcaatgctttcctcaaacaagctgctaaatcccgcagggaaaatgaggttttgaaagaaatggtgaagaaaatcagaaaagtggtgaatgatgctgaggattcaattgataaatttgtgattgaagctaagagacatgatgataaaaacaaatttgctcaatggtttcatattactcatgtggctagagcaaaaggggtagcagatgagatcaaaagtataaaggaaagagtgaaggaaattagagaaaatgacgcttatggccttcaagcgataactttagatgataatttcaatagaggtgatgaagagaggaaggcccctgtagttgaggaagatgatgtggttggttttgatgatgaagcaaaaattgtaattgatcgtctcattggaggatcagattatgttgaggttgtgccagttgttggtatgcctggtcttggtaaaacaactttggcatataagatttacaaggatccgaaggttgagtatgagttcttcacccgcgtttgggtatatgtctctcaaacgttcaagagaagggaaatatttctcaacattatcagcaagttcactcgaaacaccaaacaatatcatgatacaccagaggatgacttagcaaatgaagtgaaggagcttcttggaaaaggtggaaaatatcttattgttttggatgatgtgtggacgatggaagcttgggatcgtatcaaaattgctttccctaataatggtaaacggaatagagtgttgatgaccacgagagaatcaaatgtggctaagtgttgtaatgataaacctcatgatcttaagtttttaactgaagatgaaagttgggagctacttgagaagaaggtttttcacaaggaaaagtgtccacctgagttagaattgcccgggaaaagtatagctgaaaaatgtatgggcttacctcttgcaatagtggttatcgccggagctttgataggcaaaggaaagacaacaagggagtgggagttggtggctgccagtgtccgtgagcacctcataaatcgagatccagagaactgcaagaaattggttcaaatgagttatgatcgtttgccttatgacctaaaggcgtgttttttatactgtggggcatttccaggaggttctcaaattcctgctaagaagttgattcgtttatggatcgcggaaggattcatacaataccaaggaccattggctctagaggatgtagcagaggaccacttgaatgatcttgtcaataggaacttagtgatggtaatgcaaaggagttgcagtggtcaaatcaaaatatgtcgcgttcatgacatgttgcatgagttctgcagacacgaggctatgacagaggaggatctttttcaagaaatcaaacaagggcaagaacgatcttttccagggaaacaagagttagccacttatcgtcgtttgtgtattcattccggggttcctgaattcttatcaacaaagccctccggtgagcatgtcaggtcattcttatgtgttggctcgaagaaaattgacatgcctccaaatgaaataccatccatcccgaaagcctttccattgcttagggtactagatgctgaatcaatcaagttcaatcgattttctcgagagttcttcaaattatttcatttgaggtacattgctctctcaactgacaagattaagaccattcccgtggattttgggaatctttggaacgtacaaacacttatcgttgagacgcaagaggctactcttgacatcaaagcagacatttggaatatgacaagattaaggcatgtgtgcacaaacgcctctgctacattgccttccactaagcgccccaagagcagcaagggcaacttggtgaatcgttgcttacaaacactttcaactatagcacctgaatgttgcacagcagaagtttttactagaactcctaatctcaagaaattaggcgttcgtgggaaaatagatgcacttttagaaaccagtaaagacgggtctggttctgtcttgttcagtaacataggtaagttagcttgcctcgaatatctgaagttggtaaatgatactcgtataagtagtaaaccgctacaccttcctccagcatatattttccctcagaagctaaaaaagctctctctggtagacacttggtttgaatggaaagacatgtctatattaggtctattaccagaccttgaggtgctgaagttgaaagaaaatgcgtttaaaggacagtcgtgggagcaagaggacggtggttttcctcgtctgcaggtcttatggattgaaaggacagatttaacttcttggaaagcctcatcagggaactttccgcgactaaaacacctcgcactcatatcatgtgataagcttgaggaactcccagctgaactggctgatgtgaaaaacctccagctaattgaactccagagtagcagtgaatctgcagctagatctgcacgagcgatactaaaaaggaatcaagaaaaagaacaggaaggcgataaaggcactggattcaagctttctatattccctcatgatcttggattgtaa

SEQ ID NO: 43 - protein, 891 aa, Solanum tuberosum
MADVAVKFLVENLMQLLIDNADLILGIKGEVENLLRDLNDFNAFLKQAAKSRRENEVLKEMVKKIRKVVNDAEDSIDKFVIEAKRHDDKNKFAQWFHITHVARAKGVADEIKSIKERVKEIRENDAYGLQAITLDDNFNRGDEERKAPVVEEDDVVGFDDEAKIVIDRLIGGSDYVEVVPVVGMPGLGKTTLAYKIYKDPKVEYEFFTRVWVYVSQTFKRREIFLNIISKFTRNTKQYHDTPEDDLANEVKELLGKGGKYLIVLDDVWTMEAWDRIKIAFPNNGKRNRVLMTTRESNVAKCCNDKPHDLKFLTEDESWELLEKKVFHKEKCPPELELPGKSIAEKCMGLPLAIVVIAGALIGKGKTTREWELVAASVREHLINRDPENCKKLVQMSYDRLPYDLKACFLYCGAFPGGSQIPAKKLIRLWIAEGFIQYQGPLALEDVAEDHLNDLVNRNLVMVMQRSCSGQIKICRVHDMLHEFCRHEAMTEEDLFQEIKQGQERSFPGKQELATYRRLCIHSGVPEFLSTKPSGEHVRSFLCVGSKKIDMPPNEIPSIPKAFPLLRVLDAESIKFNRFSREFFKLFHLRYIALSTDKIKTIPVDFGNLWNVQTLIVETQEATLDIKADIWNMTRLRHVCTNASATLPSTKRPKSSKGNLVNRCLQTLSTIAPECCTAEVFTRTPNLKKLGVRGKIDALLETSKDGSGSVLFSNIGKLACLEYLKLVNDTRISSKPLHLPPAYIFPQKLKKLSLVDTWFEWKDMSILGLLPDLEVLKLKENAFKGQSWEQEDGGFPRLQVLWIERTDLTSWKASSGNFPRLKHLALISCDKLEELPAELADVKNLQLIELQSSSESAARSARAILKRNQEKEQEGDKGTGFKLSIFPHDLGL

SEQ ID NO: 44 - protein, 886 aa, Solanum tuberosum
MANVAVEFLVENLMQLLRDNAELISGVKEAAESLLQDLNDFNAFLKQAAKCHINENEVLRELVKKIRTVVNSAEDAIDKFVIEAKLHKDKGMTRVLDLPHYKRVREVAGEIKAIRNKVKEIRQNDAIGLQALQDDDSSARGFEERKPPVVEEDDVVGFDEEADIVIKRLLGESNRLEVVPVVGMPGLGKTTLANKIYKHPKIGYEFFTRIWVYVSQSYRRRELFLNIISKFTRNTKQYHGMCEEDLADEIQEFLGKGGKYLVVLDDVWSDEAWERIKIAFPNNNKPNRVLLTTRDSKVAKQCTPIPHDLKFLSEKESWILLEKKVFHKDKCPPELVVPSGKSIAKKCKGLPLAIVVIAGALIGKGKTPREWKQVDDSVSEHLINRDHPENCNKLVQMSYDRLPYDLKACFLYCSAFPGGFQIPAWKLIRLWIAEGFIQYKGHLSLECKGEDNLNDLINRNLVMVMERTSDGQIKTCRLHDMLHEFCRQEAMKEENLFQEIKLGSEQYFPGKRELSTYRRLCIHSSVLDFISTKPSAEHVRSFLSFSSKKIEMPSADIPTIPKGFPLLRVLDVESINFSRFSKEFYQLYHLRYVAFSSDSIKILPKLMGELWNIQTIIINTQQRTLDIQANIWNMERLRHLHTNSSAKLPVPVAPKNSKVTLVNQSLQTLSTIAPESCTEEVFARTPNLKKLGIRGKIAVLLDNKSAVSLKNVKRLEYLENLKLINDSSIQTGKLRLPPAYIFPTKLRKLTLLDTWLEWKDMSILGQLEHLEVLKMKENGFTGESWESTGGFCSLLVLWIERTNLVTWKASADDFPRLKHLVLICCDYLKEVPIALADIRSFQVMMLQNSTKTAAISARQIQAKKDNQTQQGTKNIAFKLSIFPPDL

SEQ ID NO: 45 - protein, 891 aa, Solanum tuberosum
MADVAVKFLVENLMQLLIDNADLILGIKGEVENLLRDLNDFNAFLKQAAKSRRENEVLKEMVKKIRKVVNDAEDSIDKFVIEAKRHDDKNKFAQWFHITHVARAKGVADEIKSIKERVKEIRENDAYGLQAITLDDNFNRGDEERKAPVVEEDDVVGFDDEAKIVIDRLIGGSDYVEVVPVVGMPGLGKTTLAYKIYKDPKVEYEFFTRVWVYVSQTFKRREIFLNIISKFTRNTKQYHDTPEDDLANEVKELLGKGGKYLIVLDDVWTMEAWDRIKIAFPNNGKRNRVLMTTRESNVAKCCNDKPHDLKFLTEKESWELLEKKVFHKEKCPPELELPGKSIAEKCMGLPLAIVVIAGALIGKGKTTREWELVAASVREHLINRDPENCKKLVQMSYDRLPYDLKACFLYCGAFPGGSQIPAKKLIRLWIAEGFIQYQGPLALEDVAEDHLNDLVNRNLVMVMQRSCSGQIKICRVHDMLHEFCRHEAMTEEDLFQEIKQGQERSFPGKQELATYRRLCIHSGVPEFLSTKPSGEHVRSFLCVGSKKIDMPPNEIPSIPKAFPLLRVLDAESIKFNRFSREFFKLFHLRYIALSTDKIKTIPVDFGNLWNVQTLIVETQEATLDIKADIWNMTRLRHVCTNASATLPSTKRPKSSKGNLVNRCLQTLSTIAPECCTAEVFTRTPNLKKLGVRGKIDALLETSKDGSGSVLFSNIGKLACLEYLKLVNDTRISSKPLHLPPAYIFPQKLKKLSLVDTWFEWKDMSILGLLPDLEVLKLKENAFKGQSWEQEDGGFPRLQVLWIERTDLTSWKASSGNFPRLKHLALISCDKLEELPAELADVKNLQLIELQSSSESAARSARAILKRNQEKEQEGDKGTGFKLSIFPHDLGL

SEQ ID NO: 46 - DNA, 2667 bp, Solanum lycopersicum
  source 1..2667: mol_type = genomic DNA
atggttgatgtaggggttgaatttctgttagagaacttgaagcaattggtactggacaatgtggagttaatcggaggagctaaagatgaaatcgagaatctgcgtgatgatttgagtgaattcaatgcctttctcaagcaagctgcaatggtccgcagcgaaaacccagttctcaaagaactagtgaggagtatcagaaaagtggtgaatcgtgctgaagatgctgttgataaatttgtaattgaagctaaagttcataaagacaaagggtttaaaggggttttcgataaacctggacattatagaagagtgagggatgcagctgtggagattaaaggtatcagagataaaatgagagaaattcggcaaaataaggcacatggccttcaggctctacttcaagatcatgatgattcaatcagcagaggtggagaagagagacagcctcctgtggttgaggaagatgatgtggtgggctttgacgatgaggcgcagacggtaatcgaccgtcttcttgaaggatcaggtgatttagaggttattccagtagttggaatgcctggtcttggcaaaactacactagccactaagatcttcaagcatccgaagattgagtacgagttctttactagactttggctttacgtttcccaatcatacaagacaagagaattatatcttaacatcatcagtaaattcaccggaaacaccaaacattgccgtgatatgtctgaaaaggatttagctcttaaggtacaagagattttggaagaaggaggaaaatacttgattgtcttggatgatgtctggtcgacagatgcttgggatcgtatcaagattgctttcccgaaaaatgacaagggcaatagagtattgttgactactcgagaccaccgtgttgcaagatattgcaataggagtccacatgatttaaaatttctgactgatgaagagagttggattttactggagaaaagagcttttcacaaagctaaatgtctccccgaattggaaacaaacggaaaaagcatagccaggaagtgtaaaggactaccccttgctattgtggtgattgcaggagctctaattgggaaaagcaaaacaataaaggaatgggagcaagtggatcagagtgtgggcgaacatttcataaatagagatcagccaaatagttgtgataaattggtacggatgagttatgatgttttgccttatgactggaaagcttgctttttatacttcggtacattccccagaggctatttaatccctgccaggaaattgatccgcttatggatcgcggaagggtttatccagtacagaggggacttatcccctgagtgtaaagcagaggagtacttgaatgaactcgtaaatagaaacttagtgatggtaatgcaaaggacggttgatggacaaatcaaaacttgtcgtgttcatgacatgttgtatgagttttgctggcaagaggctacgacagaggaaaatcttttccatgaagtaaaattcggtggtgagcaatctgttcgtgaagtatccactcatcgtcgcttgtgcattcattcctctgttgtggagttcatttctaagaagccctctggtgagcatgttaggtcgttcctatgtttttctccagaaaaaattgacactcccccaactgtcagtgcaaacatatcaaaagcctttccattgctaagggtgtttgatactgaatccatcaaaatcaatcgcttttgcaaggagttctttcaattgtatcatctgaggtatattgctttctcatttgactcgattaaagtcattccgaaacatgttggggaactttggaacgtacaaaccctcattgtcaacacacaacagatcaaccttgatattcaagcagacatattgaacatgccccggctgaggcatctgctcaccaacacgtctgctaaattgcctgcgcttgctaaccccaaaacaagtaagactaccttggtaaatcaaagcctgcaaaccctctccacaattgcaccagaaagctgcactgagtatgttctctcgagggctccaaacttgaaaaaactgggcattcgtggaaaaatagctaagctaatggaaccaagtcagtctgtattgttgaacaatgttaagaggctgcaatttcttgagaacttgaagctgataaatgttggtcagattgatcagacacaattacgccttcctccagcatctatatttccaacaaagttgaggaagctgactttattagatacctggttggagtgggatgatatgtctgtattgaaacagctggagaaccttcaagtcttgaagctgaaggacaatgcatttaagggagagaactgggaactaaatgatggaggttttcctttcctacaagtgttatgcattgaaagggcaaacttagtttcttggaatgcttcaggtgatcacttcccgagacttaaacatcttcacatatcatgtgataaacttgagaagatccccattggcctggctgatatatgcagcctccaagtgatggatttgcgaaattccactaaatcagcagcaaaatctgccagagagatacaagccaaaaaaaacaagctgcaacctgctaaatcccagaagttcgagctttctgtattccctcctgattctgatgtacagacagcttcttag

SEQ ID NO: 47 - protein, 888 aa, Solanum lycopersicum
MVDVGVEFLLENLKQLVLDNVELIGGAKDEIENLRDDLSEFNAFLKQAAMVRSENPVLKELVRSIRKVVNRAEDAVDKFVIEAKVHKDKGFKGVFDKPGHYRRVRDAAVEIKGIRDKMREIRQNKAHGLQALLQDHDDSISRGGEERQPPVVEEDDVVGFDDEAQTVIDRLLEGSGDLEVIPVVGMPGLGKTTLATKIFKHPKIEYEFFTRLWLYVSQSYKTRELYLNIISKFTGNTKHCRDMSEKDLALKVQEILEEGGKYLIVLDDVWSTDAWDRIKIAFPKNDKGNRVLLTTRDHRVARYCNRSPHDLKFLTDEESWILLEKRAFHKAKCLPELETNGKSIARKCKGLPLAIVVIAGALIGKSKTIKEWEQVDQSVGEHFINRDQPNSCDKLVRMSYDVLPYDWKACFLYFGTFPRGYLIPARKLIRLWIAEGFIQYRGDLSPECKAEEYLNELVNRNLVMVMQRTVDGQIKTCRVHDMLYEFCWQEATTEENLFHEVKFGGEQSVREVSTHRRLCIHSSVVEFISKKPSGEHVRSFLCFSPEKIDTPPTVSANISKAFPLLRVFDTESIKINRFCKEFFQLYHLRYIAFSFDSIKVIPKHVGELWNVQTLIVNTQQINLDIQADILNMPRLRHLLTNTSAKLPALANPKTSKTTLVNQSLQTLSTIAPESCTEYVLSRAPNLKKLGIRGKIAKLMEPSQSVLLNNVKRLQFLENLKLINVGQIDQTQLRLPPASIFPTKLRKLTLLDTWLEWDDMSVLKQLENLQVLKLKDNAFKGENWELNDGGFPFLQVLCIERANLVSWNASGDHFPRLKHLHISCDKLEKIPIGLADICSLQVMDLRNSTKSAAKSAREIQAKKNKLQPAKSQKFELSVFPPDSDVQTAS

SEQ ID NO: 48 - DNA, 2658 bp, Solanum lycopersicum
  source 1..2658: mol_type = genomic DNA
atggcgaacgtagcagtggaatttctggttgagaacttgatgcagttgctgcgggacaacgtagagctaattagtggagttaaagaggctgctgagagtctacttcaagatctaaatgatttcaatgcttttcttaagcaagctgccaagtgtcacatcaacgagaacgaagtcctcagagaacttgttaagaaaataagaacagtcgttaactctgctgaagatgctattgataaatttgttattgaagctaagctacacaaggataagggtgtcaccagagtattagatcttccccattataaaagagtcaaagaggtagctggtgagatcaaagctatacgaaacaaagtcagagagatccgacagactgatgccattggacttcaggcccttcaagatgatgatttatctgctagaggttccgaagaaagaaagcctccagtggtagaggaagatgacgtggttggatttgacgaagaagcagatattgtaatcaaccgccttcttggagaatcgaatcatctagaagttgttccagttgttggtatgcctggtctcggcaaaacgaccctagcaaataagatttacaagcatcctaaaatcgggtatgagttttttactcgtatttgggtttatgtgtctcaatcatacaggagaagagaattatttctcaacatcatcagcaaattcactcgaaatacaaaacaatatcatgggatgtgtgaggaggatttggctgatgaaatacaagaattcttgggaaagggaggaaaatacttggttgtgttggatgacgtatggtcggatgaagcttgggaacgtatcaagatagcattcccaaataacaacaaaccgaatcgagtattgttgaccactcgagattccaaagtggctaaacaatgcaatcccatacctcatgatctaaaatttctgactgaagatgaaagttggatattactggagaagaaggtttttcacaaggacaaatgtcctcctgaattggtactatctggaaagagcatagcaaaaaaatgcaagggactaccacttgcgattgttgttattgcaggagcactaatagggaaaggcaagacaccaagagagtggaaacaagtggatgatagtgttagtgaacacctcataaatagagaccaccctgagaattgtaacaaactggtgcaaatgagttatgatcgtttaccttacgacttgaaagcatgctttttatattgtagtgcatttcctggaggctttcaaatccctgcttggaagttaatccgtttgtggattgcggaaggattcatacagtataaaggccatttatctcttgagtgtaaaggagaggacaacttgaatgatctcatcaataggaatctagtgatggtaatggaaagaacttctgatggtcaaatcaaaacatgtcgtcttcatgacatgttgcacgagttttgcaggcaagaagctatgaaggaagaaaatcttttccaagaaataaagttaggatctgagcaatattttcctggaaaacgagaactatccacctaccgtcgcttatgcatccattcctcagttttggattttttctctacgaaaccttcagctgaacatgtcaggtcattcttatctttttcttcgaaaaagattgagatgccatctgctgacatcccaaccatacccaaaggctttccgttgctaagggttttggatgtagaatcaatcaacttcagtcgcttttccagggagttttaccagttatatcatttgaggtatgttgctttttcatctgactcaatcaagattcttcctaaactcatgggagaactttggaacatccaaaccatcataattaacacgcaacaacgcactcttgatattcaagcaaacatatggaatatggaacgactaaggcatctgcacactaactcttccgctaagttgcctgtccctgttgctccaaaaaatagtaaggttacattggtaaatcaaagcctgcaaactctctccaccatagctcccgaaagctgcaccgaggaagtgtttgcgaggactccaaacctgaaaaagctgggcatccgtgggaaaatatcagtgcttcttgataataagtctgctgcgtcgttaaaaaatgtgaagaggctagaataccttgaaaacttgaagctgataaatgatagtagcattcaaacaagtaagttacgccttccacctgcatacatatttccaacaaagttgaggaagctgactttattagacacgtggttggagtggaaagatatgtctatattgggtcagttggaacaccttgaagtcctgaagatgaaagaaaatgggttttcgggagagtcctgggagtctactggtggtttttgttccctactggtgttgtggattgaaaggacaaacttagtttcatggaaagcatcagctgatgattttccaagacttaagcatctcgttctcatctgctgtgataatcttaaggaagtccccatcgccctagctgatatacgcagtttccaagtcatgatgttgcaaaattccaccaaaacagcagcaatatctgcacgtcaaatacaagccaaaaaagacaatcaaactcaacaagggactaaaaacattgccttcaagctttccatattccctcctgatctctga

SEQ ID NO: 49 - protein, 885 aa, Solanum lycopersicum
MANVAVEFLVENLMQLLRDNVELISGVKEAAESLLQDLNDFNAFLKQAAKCHINENEVLRELVKKIRTVVNSAEDAIDKFVIEAKLHKDKGVTRVLDLPHYKRVKEVAGEIKAIRNKVREIRQTDAIGLQALQDDDLSARGSEERKPPVVEEDDVVGFDEEADIVINRLLGESNHLEVVPVVGMPGLGKTTLANKIYKHPKIGYEFFTRIWVYVSQSYRRRELFLNIISKFTRNTKQYHGMCEEDLADEIQEFLGKGGKYLVVLDDVWSDEAWERIKIAFPNNNKPNRVLLTTRDSKVAKQCNPIPHDLKFLTEDESWILLEKKVFHKDKCPPELVLSGKSIAKKCKGLPLAIVVIAGALIGKGKTPREWKQVDDSVSEHLINRDHPENCNKLVQMSYDRLPYDLKACFLYCSAFPGGFQIPAWKLIRLWIAEGFIQYKGHLSLECKGEDNLNDLINRNLVMVMERTSDGQIKTCRLHDMLHEFCRQEAMKEENLFQEIKLGSEQYFPGKRELSTYRRLCIHSSVLDFFSTKPSAEHVRSFLSFSSKKIEMPSADIPTIPKGFPLLRVLDVESINFSRFSREFYQLYHLRYVAFSSDSIKILPKLMGELWNIQTIIINTQQRTLDIQANIWNMERLRHLHTNSSAKLPVPVAPKNSKVTLVNQSLQTLSTIAPESCTEEVFARTPNLKKLGIRGKISVLLDNKSAASLKNVKRLEYLENLKLINDSSIQTSKLRLPPAYIFPTKLRKLTLLDTWLEWKDMSILGQLEHLEVLKMKENGFSGESWESTGGFCSLLVLWIERTNLVSWKASADDFPRLKHLVLICCDNLKEVPIALADIRSFQVMMLQNSTKTAAISARQIQAKKDNQTQQGTKNIAFKLSIFPPDL

SEQ ID NO: 50 - DNA, 2676 bp, Solanum lycopersicum
  source 1..2676: mol_type = genomic DNA
atggcggatgtagcagtaaagttcttattagaaaatttgacgcagttactaatcgacaacgccgatttgattctcggaatccaaggcgaagttgaaaatctactcacagatctcaattactttaatgctttcctcaaagaagctgctaaatcccgcagggaaaatgaggttttgaaagaattggtgaagaaaatcagaaaagttgtgaacgatgctgaagattcgattgataaatttgtggttgaagctaagagacatgatgataaaaacaaatttgctcaatggtttcatattactcatgtggctagagccaaaggggtagcggatgagatcaaaagtataagggaaagagtgaaggaaattagagataatgacgcttatggccttcaagctataactttggatgataatttcaacagaggtgacgaagagaggaaggcccctgtagttgaggaagatgatgtggttggttttgatgatgaagcaaaaactgtaattgatcgtctcatcggaggatcagactatgttgaggttgtgccagttgttggtatgcctggtcttggtaaaacaactttggcatataagatttacaaggatccaaaggttgagtatgagttcttcacccgcgtttgggtatatgtctctcaaacattcaagagaagggaaatatttctcaacattatcagcaagtttactcgaaacaccaaacaatatgatgatacaccagaggatgacttagcaaatgaagtgaaggagcttcttggaaaaggtggaaaatatcttattgttttggatgatgtgtggacgatggaagcttgggatcgtatcaaaattgctttccctaataatggtaaacgaaatagagtgttgatgaccacgagacaatcaaatgtggcgaagcgttgtaatgataaacctcatgatcttaagtttttaacaaaagatgaaagttgggagctacttgagaagaaggtttttcacaaggaaaagtgtccacctgagttagaattacccgggataagtatagccgaaaaatgtatgggcttacctctagctatagtggttattgccggagctttgataggcaaaggaaagacaacaagggagtgggagttggtggctgccagtgtcggtgagcacctcataaatcgagatccagagaactgcaagaaattggttcaaatgagttatgatcgtttgccttatgacctaaaggcgtgttttttatactgtggggcatttccaggaggttctcaaattccagctaagaagttgattcgtttatggatcgcggaaggattcatacaataccaaggaccattggctctggaggatgtagcagaggaccacttaaatgatcttgtcaataggaacttagtgatggtaacgcaaaggagttgcagtggtcaaatcaaaacatgtcgtgttcatgacatgttgcatgagttctgcagacacgaggccatgatggaggagaatctttttcaagaaatcaaacaagggcaagaacgatcttttccagggaaacaagagttagccacttatcgtcgtttgtgtattcaatccttgattcctgaattcttatcaatgaagccctctggtgagcatgtaaggtcattcttatgtgttggctcgaagaaaattgacatgcctccaaatgaaataccgtccatcccgaaagcctttccattgcttagggtactagatgctgaatcaatcaagttcagtcgattttctcgtgagttcttcaaattatttcatttgaggtacattgctctctcaactgacaagattaagaccattcccgcggattttgggaatctttggaacatacaaacacttatcgttgagacgcaacaggctactcttgacatcaaggcagacatttggaatatgacaagactaaggcatgtgtgcacaaacgcctcagctacattgccttctactaagcgccccaagagcagcaaggacaacttggtgaatcgatgcttacaaacactttcaactatagcaccagagtgttgcacagcagaggtttttactagaactcctaatctcaagaaattaggcgttcgtgggaaaatagatgcacttttagaatccagtaaagacgggtctggttctggcttgttcagtaacataggtaagttaggttgcctcgaatatctgaagttagtaaatgatactcgtctaagtagtaaacctctacaccttcctcctgcatatattttccctcagaagctaaaaaagctctctctggtagacacttggtttgaatggaaagatatgtctatattaggtctattaccagaacttgaggtgctgaagttgaaagaaaatgcgtttaaaggacagtcgtgggagcaagaggacggtggttttcctcgtctgcaggtcttatggattgaaaggactgatctaacttcttggaaagcctcatcagggaactttccgcgactaaaacacctcgcactcatatcatgtgataagcttgaggaactcccagctgaactggctgatgtgaaaaacctccagctaatcgaactccagagtagcagtgagtctgcagctagatctgcacgagcgatactaaaaaggaatcaagaaaaagaacaggatggcgataaaggcactggattcaagctttctatattccctcatgatcttggattgtaa

SEQ ID NO: 51 - protein, 891 aa, Solanum lycopersicum
MADVAVKFLLENLTQLLIDNADLILGIQGEVENLLTDLNYFNAFLKEAAKSRRENEVLKELVKKIRKVVNDAEDSIDKFVVEAKRHDDKNKFAQWFHITHVARAKGVADEIKSIRERVKEIRDNDAYGLQAITLDDNFNRGDEERKAPVVEEDDVVGFDDEAKTVIDRLIGGSDYVEVVPVVGMPGLGKTTLAYKIYKDPKVEYEFFTRVWVYVSQTFKRREIFLNIISKFTRNTKQYDDTPEDDLANEVKELLGKGGKYLIVLDDVWTMEAWDRIKIAFPNNGKRNRVLMTTRQSNVAKRCNDKPHDLKFLTKDESWELLEKKVFHKEKCPPELELPGISIAEKCMGLPLAIVVIAGALIGKGKTTREWELVAASVGEHLINRDPENCKKLVQMSYDRLPYDLKACFLYCGAFPGGSQIPAKKLIRLWIAEGFIQYQGPLALEDVAEDHLNDLVNRNLVMVTQRSCSGQIKTCRVHDMLHEFCRHEAMMEENLFQEIKQGQERSFPGKQELATYRRLCIQSLIPEFLSMKPSGEHVRSFLCVGSKKIDMPPNEIPSIPKAFPLLRVLDAESIKFSRFSREFFKLFHLRYIALSTDKIKTIPADFGNLWNIQTLIVETQQATLDIKADIWNMTRLRHVCTNASATLPSTKRPKSSKDNLVNRCLQTLSTIAPECCTAEVFTRTPNLKKLGVRGKIDALLESSKDGSGSGLFSNIGKLGCLEYLKLVNDTRLSSKPLHLPPAYIFPQKLKKLSLVDTWFEWKDMSILGLLPELEVLKLKENAFKGQSWEQEDGGFPRLQVLWIERTDLTSWKASSGNFPRLKHLALISCDKLEELPAELADVKNLQLIELQSSSESAARSARAILKRNQEKEQDGDKGTGFKLSIFPHDLGL

SEQ ID NO: 52 - protein, 888 aa, Solanum lycopersicum
MVDVGVEFLLENLKQLVLDNVELIGGAKDEIENLRDDLSEFNAFLKQAAMVRSENPVLKELVRSIRKVVNRAEDAVDKFVIEAKVHKDKGFKGVFDKPGHYRRVRDAAVEIKGIRDKMREIRQNKAHGLQALLQDHDDSISRGGEERQPPVVEEDDVVGFDDEAQTVIDRLLEGSGDLEVIPVVGMPGLGKTTLATKIFKHPKIEYEFFTRLWLYVSQSYKTRELYLNIISKFTGNTKHCRDMSEKDLALKVQEILEEGGKYLIVLDDVWSTDAWDRIKIAFPKNDKGNRVLLTTRDHRVARYCNRSPHDLKFLTKEESWILLEKRAFHKAKCLPELETNGKSIARKCKGLPLAIVVIAGALIGKSKTIKEWEQVDQSVGEHFINRDQPNSCDKLVRMSYDVLPYDWKACFLYFGTFPRGYLIPARKLIRLWIAEGFIQYRGDLSPECKAEEYLNELVNRNLVMVMQRTVDGQIKTCRVHDMLYEFCWQEATTEENLFHEVKFGGEQSVREVSTHRRLCIHSSVVEFISKKPSGEHVRSFLCFSPEKIDTPPTVSANISKAFPLLRVFDTESIKINRFCKEFFQLYHLRYIAFSFDSIKVIPKHVGELWNVQTLIVNTQQINLDIQADILNMPRLRHLLTNTSAKLPALANPKTSKTTLVNQSLQTLSTIAPESCTEYVLSRAPNLKKLGIRGKIAKLMEPSQSVLLNNVKRLQFLENLKLINVGQIDQTQLRLPPASIFPTKLRKLTLLDTWLEWDDMSVLKQLENLQVLKLKDNAFKGENWELNDGGFPFLQVLCIERANLVSWNASGDHFPRLKHLHISCDKLEKIPIGLADICSLQVMDLRNSTKSAAKSAREIQAKKNKLQPAKSQKFELSVFPPDSDVQTAS

SEQ ID NO: 53 - protein, 885 aa, Solanum lycopersicum
MANVAVEFLVENLMQLLRDNVELISGVKEAAESLLQDLNDFNAFLKQAAKCHINENEVLRELVKKIRTVVNSAEDAIDKFVIEAKLHKDKGVTRVLDLPHYKRVKEVAGEIKAIRNKVREIRQTDAIGLQALQDDDLSARGSEERKPPVVEEDDVVGFDEEADIVINRLLGESNHLEVVPVVGMPGLGKTTLANKIYKHPKIGYEFFTRIWVYVSQSYRRRELFLNIISKFTRNTKQYHGMCEEDLADEIQEFLGKGGKYLVVLDDVWSDEAWERIKIAFPNNNKPNRVLLTTRDSKVAKQCNPIPHDLKFLTEKESWILLEKKVFHKDKCPPELVLSGKSIAKKCKGLPLAIVVIAGALIGKGKTPREWKQVDDSVSEHLINRDHPENCNKLVQMSYDRLPYDLKACFLYCSAFPGGFQIPAWKLIRLWIAEGFIQYKGHLSLECKGEDNLNDLINRNLVMVMERTSDGQIKTCRLHDMLHEFCRQEAMKEENLFQEIKLGSEQYFPGKRELSTYRRLCIHSSVLDFFSTKPSAEHVRSFLSFSSKKIEMPSADIPTIPKGFPLLRVLDVESINFSRFSREFYQLYHLRYVAFSSDSIKILPKLMGELWNIQTIIINTQQRTLDIQANIWNMERLRHLHTNSSAKLPVPVAPKNSKVTLVNQSLQTLSTIAPESCTEEVFARTPNLKKLGIRGKISVLLDNKSAASLKNVKRLEYLENLKLINDSSIQTSKLRLPPAYIFPTKLRKLTLLDTWLEWKDMSILGQLEHLEVLKMKENGFSGESWESTGGFCSLLVLWIERTNLVSWKASADDFPRLKHLVLICCDNLKEVPIALADIRSFQVMMLQNSTKTAAISARQIQAKKDNQTQQGTKNIAFKLSIFPPDL

SEQ ID NO: 54 - protein, 891 aa, Solanum lycopersicum; identical to SEQ ID NO: 51

SEQ ID NO: 55 - protein, 886 aa, Solanum tuberosum
MANVAVEFLVENLMQLLRDNAELISGVKEAAESLLQDLNDFNAFLKQAAKCHINENEVLRELVKKIRTVVNSAEDAIDKFVIEAKLHKDKGMTRVLDLPHYKRVREVAGEIKAIRNKVKEIRQNDAIGLQALQDDDSSARGFEERKPPVVEEDDVVGFDEEADIVIKRLLGESNRLEVVPVVGMPGLGKTTLANKIYKHPKIGYEFFTRIWVYVSQSYRRRELFLNIISKFTRNTKQYHGMCEEDLADEIQEFLGKGGKYLVVLDDVWSDEAWERIKIAFPNNNKPNRVLLTTRDSKVAKQCTPIPHDLKFLSEDEKWILLEKKVFHKDKCPPELVVPSGKSIAKKCKGLPLAIVVIAGALIGKGKTPREWKQVDDSVSEHLINRDHPENCNKLVQMSYDRLPYDLKACFLYCSAFPGGFQIPAWKLIRLWIAEGFIQYKGHLSLECKGEDNLNDLINRNLVMVMERTSDGQIKTCRLHDMLHEFCRQEAMKEENLFQEIKLGSEQYFPGKRELSTYRRLCIHSSVLDFISTKPSAEHVRSFLSFSSKKIEMPSADIPTIPKGFPLLRVLDVESINFSRFSKEFYQLYHLRYVAFSSDSIKILPKLMGELWNIQTIIINTQQRTLDIQANIWNMERLRHLHTNSSAKLPVPVAPKNSKVTLVNQSLQTLSTIAPESCTEEVFARTPNLKKLGIRGKIAVLLDNKSAVSLKNVKRLEYLENLKLINDSSIQTGKLRLPPAYIFPTKLRKLTLLDTWLEWKDMSILGQLEHLEVLKMKENGFTGESWESTGGFCSLLVLWIERTNLVTWKASADDFPRLKHLVLICCDYLKEVPIALADIRSFQVMMLQNSTKTAAISARQIQAKKDNQTQQGTKNIAFKLSIFPPDL

SEQ ID NO: 56 - protein, 891 aa, Solanum tuberosum
MADVAVKFLVENLMQLLIDNADLILGIKGEVENLLRDLNDFNAFLKQAAKSRRENEVLKEMVKKIRKVVNDAEDSIDKFVIEAKRHDDKNKFAQWFHITHVARAKGVADEIKSIKERVKEIRENDAYGLQAITLDDNFNRGDEERKAPVVEEDDVVGFDDEAKIVIDRLIGGSDYVEVVPVVGMPGLGKTTLAYKIYKDPKVEYEFFTRVWVYVSQTFKRREIFLNIISKFTRNTKQYHDTPEDDLANEVKELLGKGGKYLIVLDDVWTMEAWDRIKIAFPNNGKRNRVLMTTRESNVAKCCNDKPHDLKFLTEDEKWELLEKKVFHKEKCPPELELPGKSIAEKCMGLPLAIVVIAGALIGKGKTTREWELVAASVREHLINRDPENCKKLVQMSYDRLPYDLKACFLYCGAFPGGSQIPAKKLIRLWIAEGFIQYQGPLALEDVAEDHLNDLVNRNLVMVMQRSCSGQIKICRVHDMLHEFCRHEAMTEEDLFQEIKQGQERSFPGKQELATYRRLCIHSGVPEFLSTKPSGEHVRSFLCVGSKKIDMPPNEIPSIPKAFPLLRVLDAESIKFNRFSREFFKLFHLRYIALSTDKIKTIPVDFGNLWNVQTLIVETQEATLDIKADIWNMTRLRHVCTNASATLPSTKRPKSSKGNLVNRCLQTLSTIAPECCTAEVFTRTPNLKKLGVRGKIDALLETSKDGSGSVLFSNIGKLACLEYLKLVNDTRISSKPLHLPPAYIFPQKLKKLSLVDTWFEWKDMSILGLLPDLEVLKLKENAFKGQSWEQEDGGFPRLQVLWIERTDLTSWKASSGNFPRLKHLALISCDKLEELPAELADVKNLQLIELQSSSESAARSARAILKRNQEKEQEGDKGTGFKLSIFPHDLGL

SEQ ID NO: 57 - protein, 888 aa, Solanum lycopersicum
MVDVGVEFLLENLKQLVLDNVELIGGAKDEIENLRDDLSEFNAFLKQAAMVRSENPVLKELVRSIRKVVNRAEDAVDKFVIEAKVHKDKGFKGVFDKPGHYRRVRDAAVEIKGIRDKMREIRQNKAHGLQALLQDHDDSISRGGEERQPPVVEEDDVVGFDDEAQTVIDRLLEGSGDLEVIPVVGMPGLGKTTLATKIFKHPKIEYEFFTRLWLYVSQSYKTRELYLNIISKFTGNTKHCRDMSEKDLALKVQEILEEGGKYLIVLDDVWSTDAWDRIKIAFPKNDKGNRVLLTTRDHRVARYCNRSPHDLKFLTDKESWILLEKRAFHKAKCLPELETNGKSIARKCKGLPLAIVVIAGALIGKSKTIKEWEQVDQSVGEHFINRDQPNSCDKLVRMSYDVLPYDWKACFLYFGTFPRGYLIPARKLIRLWIAEGFIQYRGDLSPECKAEEYLNELVNRNLVMVMQRTVDGQIKTCRVHDMLYEFCWQEATTEENLFHEVKFGGEQSVREVSTHRRLCIHSSVVEFISKKPSGEHVRSFLCFSPEKIDTPPTVSANISKAFPLLRVFDTESIKINRFCKEFFQLYHLRYIAFSFDSIKVIPKHVGELWNVQTLIVNTQQINLDIQADILNMPRLRHLLTNTSAKLPALANPKTSKTTLVNQSLQTLSTIAPESCTEYVLSRAPNLKKLGIRGKIAKLMEPSQSVLLNNVKRLQFLENLKLINVGQIDQTQLRLPPASIFPTKLRKLTLLDTWLEWDDMSVLKQLENLQVLKLKDNAFKGENWELNDGGFPFLQVLCIERANLVSWNASGDHFPRLKHLHISCDKLEKIPIGLADICSLQVMDLRNSTKSAAKSAREIQAKKNKLQPAKSQKFELSVFPPDSDVQTAS

SEQ ID NO: 58 - protein, 885 aa, Solanum lycopersicum
MANVAVEFLVENLMQLLRDNVELISGVKEAAESLLQDLNDFNAFLKQAAKCHINENEVLRELVKKIRTVVNSAEDAIDKFVIEAKLHKDKGVTRVLDLPHYKRVKEVAGEIKAIRNKVREIRQTDAIGLQALQDDDLSARGSEERKPPVVEEDDVVGFDEEADIVINRLLGESNHLEVVPVVGMPGLGKTTLANKIYKHPKIGYEFFTRIWVYVSQSYRRRELFLNIISKFTRNTKQYHGMCEEDLADEIQEFLGKGGKYLVVLDDVWSDEAWERIKIAFPNNNKPNRVLLTTRDSKVAKQCNPIPHDLKFLTEDEKWILLEKKVFHKDKCPPELVLSGKSIAKKCKGLPLAIVVIAGALIGKGKTPREWKQVDDSVSEHLINRDHPENCNKLVQMSYDRLPYDLKACFLYCSAFPGGFQIPAWKLIRLWIAEGFIQYKGHLSLECKGEDNLNDLINRNLVMVMERTSDGQIKTCRLHDMLHEFCRQEAMKEENLFQEIKLGSEQYFPGKRELSTYRRLCIHSSVLDFFSTKPSAEHVRSFLSFSSKKIEMPSADIPTIPKGFPLLRVLDVESINFSRFSREFYQLYHLRYVAFSSDSIKILPKLMGELWNIQTIIINTQQRTLDIQANIWNMERLRHLHTNSSAKLPVPVAPKNSKVTLVNQSLQTLSTIAPESCTEEVFARTPNLKKLGIRGKISVLLDNKSAASLKNVKRLEYLENLKLINDSSIQTSKLRLPPAYIFPTKLRKLTLLDTWLEWKDMSILGQLEHLEVLKMKENGFSGESWESTGGFCSLLVLWIERTNLVSWKASADDFPRLKHLVLICCDNLKEVPIALADIRSFQVMMLQNSTKTAAISARQIQAKKDNQTQQGTKNIAFKLSIFPPDL

SEQ ID NO: 59 - protein, 891 aa, Solanum lycopersicum
MADVAVKFLLENLTQLLIDNADLILGIQGEVENLLTDLNYFNAFLKEAAKSRRENEVLKELVKKIRKVVNDAEDSIDKFVVEAKRHDDKNKFAQWFHITHVARAKGVADEIKSIRERVKEIRDNDAYGLQAITLDDNFNRGDEERKAPVVEEDDVVGFDDEAKTVIDRLIGGSDYVEVVPVVGMPGLGKTTLAYKIYKDPKVEYEFFTRVWVYVSQTFKRREIFLNIISKFTRNTKQYDDTPEDDLANEVKELLGKGGKYLIVLDDVWTMEAWDRIKIAFPNNGKRNRVLMTTRQSNVAKRCNDKPHDLKFLTKDEKWELLEKKVFHKEKCPPELELPGISIAEKCMGLPLAIVVIAGALIGKGKTTREWELVAASVGEHLINRDPENCKKLVQMSYDRLPYDLKACFLYCGAFPGGSQIPAKKLIRLWIAEGFIQYQGPLALEDVAEDHLNDLVNRNLVMVTQRSCSGQIKTCRVHDMLHEFCRHEAMMEENLFQEIKQGQERSFPGKQELATYRRLCIQSLIPEFLSMKPSGEHVRSFLCVGSKKIDMPPNEIPSIPKAFPLLRVLDAESIKFSRFSREFFKLFHLRYIALSTDKIKTIPADFGNLWNIQTLIVETQQATLDIKADIWNMTRLRHVCTNASATLPSTKRPKSSKDNLVNRCLQTLSTIAPECCTAEVFTRTPNLKKLGVRGKIDALLESSKDGSGSGLFSNIGKLGCLEYLKLVNDTRLSSKPLHLPPAYIFPQKLKKLSLVDTWFEWKDMSILGLLPELEVLKLKENAFKGQSWEQEDGGFPRLQVLWIERTDLTSWKASSGNFPRLKHLALISCDKLEELPAELADVKNLQLIELQSSSESAARSARAILKRNQEKEQDGDKGTGFKLSIFPHDLGL

SEQ ID NO: 60 - protein, 88 aa, Nicotiana benthamiana
RDDKTHRPELLSPDNSWLFCNVAFAANDGTCERPELEDVGKEIVTKCKGLPLTIKAVGGLLLCKDHVYHEWRRIAEHFQDELRGNTSE

SEQ ID NO: 61 - protein, 84 aa, Nicotiana benthamiana
CIGIPHDLKFTEDESWILLEKKVFHKDKCPPELELSGKSIAKKCNGLPLAIVVIAGALIGKGKTSREWKQVDESVGEHLINKDQ

SEQ ID NO: 62 - protein, 83 aa, Nicotiana benthamiana
ANSDPHDLKFLTPKESFELLVKRVFGKKPCPKDLVGHGESIAGKCGGVPLAVVVIAGALRGRPNTSDWIRVERNVVQHLYTNS

SEQ ID NO: 63 - protein, 48 aa, Solanum lycopersicum
RDDKTHRPELLSPDNSWLLFCNVAFAANDGTCERPELEDVGKEIVTKC

SEQ ID NO: 64 - protein, 45 aa, Solanum lycopersicum
CNRSPHDLKFLTDEESWILLEKRAFHKAKCLPELETNGKSIARKC

SEQ ID NO: 65 - protein, 45 aa, Nicotiana benthamiana
CIGIPHDLKFLTEDESWILLEKKVFHKDKCPPELELSGKSIAKKC

SEQ ID NO: 66 - protein, 45 aa, Nicotiana benthamiana
CNDKPHDLKFLTENESWELLEKRVFHKEKCPFELELPGKSIAKKC

SEQ ID NO: 67 - protein, 45 aa, Nicotiana benthamiana
ANSDPHDLKFLTPKESFELLVKRVFGKKPCPKDLVGHGESIAGKC

(19) United States
(12) Patent Application Publication
(10) Pub. No.: US 2026/0201405 A1
(43) Pub. Date: Jul. 16, 2026

(54) METHODS OF MODULATING IMMUNE FUNCTION IN PLANTS

(71) Applicant: RESURRECT BIO LTD, London (GB)

(72) Inventors: Sophien Kamoun, Norwich (GB); Lida Derevnina, London (GB); Mauricio Contreras, London (GB)

(21) Appl. No.: 19/134,828

(22) PCT Filed: Dec. 5, 2023
(86) PCT No.: PCT/EP2023/084349

§ 371 Date: Jun. 2, 2025

(30) Foreign Application Priority Data
Dec. 5, 2022 (LU) 503137

(51) Int. Cl. C12N 15/82; C07K 14/415

(52) U.S. Cl. CPC C12N 15/8285; C07K 14/415; C12N 15/8216

(57) ABSTRACT

The present invention relates to genetically altered plants, parts thereof and plant cells that comprise one or more mutations in one or more NLR proteins, such as the helper NLR proteins, NRC2 and/or NRC3, as well as methods of providing or improving immunity of a plant to a pathogen or pest by introducing one or more mutations into one or more NLR genes.

### TECHNICAL FIELD

The present invention relates to genetically altered plants, parts thereof and plant cells that comprise one or more mutations in one or more NLR proteins, such as the helper NLR proteins, NRC2 and/or NRC3, as well as methods of providing or improving immunity of a plant to a pathogen or pest by introducing one or more mutations into one or more NLR genes.

### BACKGROUND

Plant diseases and pests threaten agricultural productivity and food quality and pose a clear and present danger to our food systems. Up to 30% of global crop yields can be lost due to pathogens and pests, costing hundreds of billions of dollars in lost food production. The amount lost would be enough to feed several billion people. By 2050, global food production is estimated to require a ~70% increase to feed the expected human population.

Outbreaks caused by plant diseases have increased in frequency due to global trade, climate change, and the propensity of plant pathogens to break down the disease resistance that had been painstakingly bred into crop plants. Current crop disease management strategies rely on chemical control, but many of the popular fungicides/pesticides face increasingly rigorous regulations due to environmental and health concerns. The most sustainable strategy to manage plant pathogens and pests is to breed broad-spectrum disease resistance crops. However, current disease resistance breeding approaches can be slow and inefficient and haven't fully tapped into our fundamental understanding of plant immunity.

A sustainable way to combat pathogens is through genetic improvement of crops. Although plants have the genetic toolkit to fight diseases, the capacity of pathogens to adapt and evade the plant immune system has constrained resistance breeding. Plants defend against parasites through specialized disease resistance proteins that are encoded as immune sensors, which activate plant immunity upon sensing pathogens. Yet, some pathogens evade or suppress plant disease resistance, limiting the potency of these immune sensors in agriculture. Engineering of disease resistance functions is an alternative strategy but historically has been limited by our superficial understanding of underlying mechanisms.

There therefore exists a need to develop new genetic mechanisms to provide disease resistance to plants, and in particular to the commercially important crops.

The nucleotide binding and leucine-rich repeat (NLR) class of intracellular immune receptors are an important component of innate immunity in plants and animals. They mediate intracellular recognition of pathogens and subsequently initiate an array of immune responses in order to counteract infection. NLRs can be activated by pathogen-secreted virulence proteins, termed effectors, which pathogens deliver into host cells to modulate host physiology. A hallmark of plant and animal NLR activation is their oligomerization into higher order immune complexes termed resistosomes or inflammasomes, respectively. These complexes initiate immune signaling via diverse mechanisms, often leading to a form of programmed cell death, termed hypersensitive response (HR) in plants or pyroptosis in animals. Recent studies have reported NLR-like proteins mediating antiviral immunity and programmed cell death in prokaryotes via mechanisms analogous to those found in eukaryotic NLRs, suggesting that this is a conserved defense mechanism across all three domains of life. Remarkably, pathogen effectors can act both as triggers and suppressors of NLR-mediated immunity. In some cases, adapted pathogens deploy effectors which directly or indirectly interfere with NLR signaling via diverse strategies to suppress immune activation. The exact mechanisms by which pathogen effectors can compromise NLR-mediated immunity to promote disease remain largely unknown. Moreover, while multiple strategies to engineer novel effector recognition specificities in NLRs have been proposed in recent years, approaches to mitigate the impact of effector-mediated immune suppression of NLRs are lacking.

### SUMMARY

The technology described herein allows for the engineering of disease resistant crops with minimal changes to the genome, thus increasing food production whilst minimising chemical usage. Up until now, it has been challenging to determine ways of using modern breeding techniques to improve disease resistance, therefore the invention presented here represents a beneficial tool to support farmers in their fight against plant diseases and help their struggle to feed the world.

The following embodiments apply to all aspects of the invention.

In one aspect of the invention, there is provided a genetically altered plant, plant part thereof or plant cell, wherein the plant, part thereof or plant cell comprises at least one mutation in at least one nucleic acid sequence encoding a nucleotide-binding domain and leucine-rich repeat-containing (NLR) protein, wherein the NLR protein comprises a HD1 domain, and wherein the at least one mutation is a mutation of one or more amino acids in an N-terminal portion of the HD1 domain.

In one embodiment, the at least one mutation reduces or prevents inhibition by a pathogen effector of initiation of an immune response by the NLR protein.

In another aspect of the invention, there is provided a genetically altered plant, plant part thereof or plant cell, wherein the plant, part thereof or plant cell comprises at least one mutation in at least one nucleic acid sequence encoding a nucleotide-binding domain and leucine-rich repeat-containing (NLR) protein, wherein the NLR protein initiates an immune response pathway, and wherein the at least one mutation reduces or prevents the inhibition by a pathogen effector of the initiation of said immune response pathway by the NLR protein.

In another aspect of the invention, there is provided a method of providing or improving pathogen resistance in a plant, the method comprising introducing at least one mutation into at least one nucleic acid sequence encoding a nucleotide-binding domain and leucine-rich repeat-containing (NLR) protein, wherein the NLR protein comprises a HD1 domain; and wherein the at least one mutation is a mutation of one or more amino acids in an N-terminal portion of the HD1 domain.

In another aspect of the invention, there is provided a method of providing or improving pathogen resistance in a plant, the method comprising introducing and expressing a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

In another aspect of the invention, there is provided a method of producing a plant with improved pathogen resistance, the method comprising introducing at least one mutation into at least one nucleic acid sequence encoding a nucleotide-binding domain and leucine-rich repeat-containing (NLR) protein, wherein the NLR protein comprises a HD1 domain; and wherein the at least one mutation is a mutation of one or more amino acids in an N-terminal portion of the HD1 domain.

In another aspect of the invention, there is provided a method of producing a plant with improved pathogen resistance, the method comprising introducing and expressing a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

In another aspect of the invention, there is provided a method of producing an altered NLR protein, wherein a pathogen effector is either unable or has reduced ability to bind to the NLR protein, wherein the method comprises introducing at least one mutation into at least one nucleic acid sequence encoding an NLR protein, wherein the NLR protein comprises a HD1 domain; and wherein the at least one mutation is a mutation of one or more amino acids in an N-terminal portion of the HD1 domain.

In one embodiment, the NLR protein initiates the immune response by interacting with one or more downstream signalling partners, and the at least one mutation preserves said interaction(s).

In another aspect of the invention, there is provided a genetically altered plant, plant part thereof or plant cell, wherein the plant, part thereof or plant cell expresses a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

In one embodiment, the NLR protein is a nucleotide-binding domain and leucine-rich repeat-containing required for cell-death (NRC) protein.

In one embodiment, the at least one mutation reduces or prevents inhibition by a pathogen effector of oligomerization of the NRC protein into a complex for initiating an immune response.

In one embodiment, the mutation reduces or prevents binding between the NLR protein and the pathogen effector.

In one embodiment, the N-terminal portion of the HD1 domain comprises no more than the N-terminal 42 amino acids of the HD1 domain, more preferably no more than the N-terminal 38 amino acids of the HD1 domain.

In one embodiment, the N-terminal portion of the HD1 domain comprises a sequence as defined in SEQ ID NO: 2, 5, 8, 14, 17, 23, 26 or 29 or a variant or fragment thereof. Preferably, the variant has at least 60% overall sequence identity to SEQ ID NO: 2, 5, 8, 14, 17, 23, 26 or 29.

In one embodiment, the at least one mutation is in a RNBS-C motif of the HD1 domain. Preferably, the RNBS-C motif comprises a sequence as defined in SEQ ID NO: 3, 6, 9, 15, 18, 24, 27 or 30 or variant or fragment thereof. More preferably, the variant has at least 60% overall sequence identity to the sequence defined in SEQ ID NO: 3, 6, 9, 15, 18, 24, 27 or 30.

In one embodiment, the mutation is a substitution.

Preferably, the mutation is a substitution at one or more positions in the amino acid sequence, where the position is selected from 315, 316 or 317 of any of SEQ ID NO: 32, 34, 36, 41, 43, 47, 79 or 51 or a homolog or functional variant thereof. Alternatively, the substitution is a homologous position in a homologous sequence.

In one embodiment, the substitution is a substitution to a hydrophilic amino acid and/or a positively charged amino acid.

In one embodiment, the mutation is a substitution of a D residue. Alternatively, the mutation is a substitution of an E residue. Alternatively, the mutation is a substitution of an N residue. Alternatively, the mutation is a substitution of an S residue.

In one embodiment, the substitution is a D317K substitution or a homologous position in a homologous sequence in SEQ ID NO: 32 or 34 or a homologue or functional variant thereof.

In one embodiment, the substitution is a N317K substitution or a homologous position in a homologous sequence in SEQ ID NO: 36 or a homologue or functional variant thereof.

In one embodiment, the substitution is a D315K substitution or a homologous position in a homologous sequence in SEQ ID NO: 41, 43, 49 or 51 or a homologue or functional variant thereof.

In one embodiment, the substitution is a D316K substitution or a homologous position in a homologous sequence in SEQ ID NO: 47, or a homologue or functional variant thereof.

In one embodiment, the substitution is a S317K substitution or a homologous position in a homologous sequence in SEQ ID NO: 41, 43, 49 or 51 or a homologue or functional variant thereof.

In one embodiment, the substitution is a E317K substitution or a homologous position in a homologous sequence in SEQ ID NO: 47 or a homologue or functional variant thereof.

Preferably, as shown in FIG. 5, the substitution is preferably at position D317 of SEQ ID NO: 32 or 34 or a homologous position in a homologous sequence, wherein preferably the substitution is D317K.

In one embodiment, the mutation is a substitution of all or a proportion of the N-terminal portion of the HD1 domain for a corresponding portion in a second NLR protein, wherein the second NLR protein is not inhibited by the pathogen effector.

In one embodiment, the mutation is a substitution of all or a significant proportion of the RNBS-C motif for a corresponding RNBS-C motif in a second NLR protein, wherein the second NLR protein is not inhibited by the pathogen effector.

In one embodiment, the nucleic acid construct expresses a nucleic acid sequence that encodes a NLR protein, wherein the NLR protein is selected from SEQ ID NO: 37, 38, 39, 44, 45, 52, 53, 54, 55, 56, 57, 58, or 59, and wherein preferably the regulatory sequence is operably linked to a regulatory sequence.

In one embodiment, the pathogen is a potato cyst nematode. Preferably, the pathogen effector is SPRYSEC15.

In one embodiment, the plant is a monocot or dicot. Preferably, the plant is a crop plant. More preferably, the plant is a solanaceous plant.

In one embodiment, the NLR protein is NRC1 and/or NRC2 and/or NRC3.

In one embodiment, the plant part is a seed or grain.

In another aspect of the invention, there is provided a plant obtained or obtainable by the method of the invention.

In another aspect of the invention, there is provided method of screening a population of plants and identifying and/or selecting a plant that will exhibit pathogen resistance or improved pathogen resistance, the method comprising detecting in the plant or plant germplasm at least one polymorphism in a NRC2 and/or NRC3 gene, wherein preferably the polymorphism is in an N-terminal portion of the HD1 domain of the NRC2 and/or NRC3 gene.

In one embodiment, the N-terminal portion of the HD1 domain comprises no more than the N-terminal 42 amino acids of the HD1 domain, more preferably no more than the N-terminal 38 amino acids of the HD1 domain.

In another aspect of the invention, there is provided, an isolated nucleotide-binding domain and leucine-rich repeat-containing (NLR) protein, wherein the NLR protein comprises a sequence selected from SEQ ID NO: 37, 38, 39, 44, 45, 52, 53 54, 55, 56, 57, 58, 59, or a functional variant or homologue thereof.

### BRIEF DESCRIPTION OF THE DRAWINGS

The invention is further described in the following non-limiting figures:

FIG. 1: SS15 directly inhibits NRC2 formation.

(A) Schematic representation of an NRC immune receptor network, comprising multiple sensor NLRs (Prf, Gpa2, Rx, R1 and Rpi-blb2) and their downstream helper NLRs, NRC2 and NRC4. Effector-triggered activation of one or more of the sensors leads to downstream helper oligomerization and resistosome formation. The Globodera rostochiensis effector SS15 can directly inhibit NRC2 by directly binding to the NB-ARC domain. (B) BN-PAGE assays with inactive and activated Rx together with NRC2 or NRC4, in the absence or presence of SS15. C-terminally V5-tagged Rx and C-terminally 4×Myc tagged NRC2^{EEE} or NRC4^{AAA} were co-expressed with either free GFP or C-terminally GFP-tagged PVX CP (potato virus X coat protein). These effector-sensor-helper combinations were co-infiltrated together with a 6×HA-mCherry fusion protein or with N-terminally 6×HA-tagged SS15. Total protein extracts were run on native and denaturing PAGE assays in parallel and immunoblotted with the appropriate antisera labelled below. Approximate molecular weights (kDa) of the proteins are shown on the left. The experiment was repeated three times with similar results.

FIG. 2: The HD1-1 region of the NB-ARC domain determines SS15 association and inhibition of NRCs.

(A) Schematic representation of NRC domain architecture, highlighting regions within the NB-ARC domain swapped between NRC2-NRC4 chimeric proteins, namely NRC4^{2HD1-1} and NRC4^{2HD1-2}). Association (+) or lack thereof (−) between these NLR immune receptors and SS15 by in planta co-immunoprecipitation is detailed on the right.

(B) Close-up view of amino acid sequence alignment between AtZAR1, NRC2 and NRC4 (N. benthamiana) focused on the HD1 region of the NB-ARC domain. Predicted secondary structure is shown above the alignment. Well-characterized motifs within this region, such as RNBS-C and GLPL are underlined below the alignment. (C) Co-Immunoprecipitation (Co-IP) assays between SS15 and chimeric NRC2-NRC4 variants. C-terminally 4×Myc-tagged NRC proteins were transiently co-expressed with N-terminally 4×HA-tagged SS15. IPs were performed with agarose beads conjugated to Myc antibodies (Myc IP). Total protein extracts were immunoblotted with the appropriate antisera labelled on the left. Approximate molecular weights (kDa) of the proteins are shown on the right. Rubisco loading control was carried out using Ponceau stain (PS). The experiment was repeated three times with similar results. (D) Photo of representative leaves from N. benthamiana nrc2/3/4 KO plants showing HR after co-expression of Rx and PVX CP with NRC2, NRC4 and two NRC2-NRC4 chimeras, NRC42HD1-1 and NRC42HD1-2. These effector-sensor-helper combinations were co-expressed with a free mCherry-6×HA fusion protein (EV) or with N-terminally 4×HA-tagged SS15. (E) BN-PAGE assay with inactive and activated Rx together with NRC4 or an NRC2-NRC4 chimeric protein in the absence or presence of SS15. C-terminally V5-tagged Rx and C-terminally 4×Myc tagged NRC4^{AAA} or NRC4^{AAA-2HD1-1} were co-expressed with either free GFP or C-terminally GFP-tagged PVX CP. These effector-sensor-helper combinations were co-infiltrated together with a mCherry-6×HA fusion protein or with N-terminally 4×HA-tagged SS15. Total protein extracts were run on native and denaturing PAGE assays in parallel and immunoblotted with the appropriate antisera labelled on the left. Approximate molecular weights (kDa) of the proteins are shown on the left. The experiment was repeated three times with similar results.

FIG. 3: Identification of SS15-NRC binding interface enables engineering of NRC2 to evade pathogen suppression.

(A) Structure of the SS15-NRC1^{NB-ARC} complex (B) Alignment of HD1-1 region of AtZAR1, NRC1 (tomato), NRC2, NRC3, and NRC4 (N. benthamiana). Candidate residues were shortlisted based on the interface identified in the co-crystal structure of SS15 and the NRC1 NB-ARC domain, as well as being conserved in NRC1, NRC2 and NRC3 but not NRC4 and AtZAR1. 13 NRC2 variants were generated by mutating individual candidate positions to the corresponding amino acid in NRC4 (detailed underneath the alignment). (C) Photo of representative leaves from N. benthamiana nrc2/3/4 KO plants showing HR after co-expression of Rx and PVX CP with NRC2, or different NRC2 variants generated. These effector-sensor-helper combinations were co-expressed with a free mCherry-6×HA fusion protein (EV) or with N-terminally 4×HA-tagged SS15. (D) Co-Immunoprecipitation (Co-IP) assays between SS15 and NRC2 variants. C-terminally 4×Myc-tagged NRC2 variants were transiently co-expressed with N-terminally 4×HA-tagged SS15. IPs were performed with agarose beads conjugated to Myc antibodies (Myc IP). Total protein extracts were immunoblotted with appropriate antisera labelled on the left. Approximate molecular weights (kDa) of the proteins are shown on the right. Rubisco loading control was carried out using Ponceau stain (PS). The experiment was repeated three times with similar results.

FIG. 4: Crystal structure of SS15 in complex with NRC1NB-ARC

(A) Electron density map showing the relative orientation and arrangement of SS15 and NRC1^{NB-ARC} within an asymmetric unit in tomato. 2Fo-Fc map countered at 10. (B) Two possible interfaces between SS15 and NRC1^{NB-ARC} revealed from the crystal packing. Both interfaces (Interface 1 and Interface 2) are outlined (Left). Modelling of both potential binding interfaces for SS15 complex with full length NRC1 reveals a steric clash between the CC-domain of NRC1 and SS15, making interface 2 unlikely to be biologically relevant in the full-length context (Right). (C) Close up view of interaction between SS15-NRC1NB-ARC interaction interface relative to the ATP-binding site within the NB-ARC domain of NRC1. The phosphate moiety of ATP is oriented facing opposite the SS15 binding interface (shown as ball and sticks, suggesting that SS15 is unlikely to displace bound ATP or prevent ATP hydrolysis. (D) Structure of SS15-NRC1NB-ARC is superimposed over the NB-ARC domain of AtZAR1 in its inactive, intermediate, and active conformations. Visualizing these three states reveals the trajectory of the NB domain as it moves relative to the HD1 and WHD domains while changing from inactive to activated states. Without wishing to be bound by theory, the binding of SS15 at the critical hinge region between the NB and HD1-WHD domains likely immobilizes this loop, preventing these critical intramolecular rearrangements and therefore preventing NLR activation.

FIG. 5: The engineered NRC2^{D317K} helper supports immune signaling of multiple sensor NLRs in the presence of SS15.

(A) Photo of representative leaves from N. benthamiana nrc2/3/4 KO plants showing HR after co-expression of different NRC2-dependent sensor NLRs together with NRC2, NRC2^{E316P} or NRC2^{D317K} in the absence or presence of SS15. (B) BN-PAGE assays with inactive and activated Rx together with NRC2 or NRC2^{D317K}, in the absence or presence of SS15. C-terminally V5-tagged Rx and C-terminally 4×Myc tagged NRC2^{EEE} or NRC2^{EEE-D317K} were co-expressed with either free GFP or C-terminally GFP-tagged PVX CP. These effector-sensor-helper combinations were co-infiltrated together with a 6×HA-mCherry fusion protein or with N-terminally 4×HA-tagged SS15. Total protein extracts were run on native and denaturing PAGE assays in parallel and immunoblotted with the appropriate antisera labelled below. Approximate molecular weights (kDa) of the proteins are shown on the left. The experiment was repeated three times with similar results.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics, which are within the skill of the art. Such techniques are explained fully in the literature.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

For the purposes of the invention, a “genetically altered plant” is a plant that has been genetically altered compared to the naturally occurring wild type (WT) plant. In one embodiment, a genetically altered plant is a plant that has been altered compared to the naturally occurring wild type (WT) plant using a mutagenesis method, such as targeted genome modification or genome editing. In one embodiment, the plant genome has been altered compared to the wild-type using a mutagenesis method. Such plants have an altered phenotype as described herein, such as increased immunity to a pathogen. Therefore, in this example, these phenotypes are conferred by the presence of an altered plant genome, for example the mutation of at least one gene encoding an NLR gene. In particular, the aspects of the invention involve recombination DNA technology and exclude embodiments that are solely based on generating plants by traditional breeding methods.

According to all aspects of the invention, including the method below and including the plants, methods and uses as described below, the term “regulatory sequence” is used interchangeably herein with “promoter” and all terms are to be taken in a broad context to refer to regulatory nucleic acid sequences capable of effecting expression of the sequences to which they are ligated. The term “regulatory sequence” also encompasses a synthetic fusion molecule or derivative that confers, activates or enhances expression of a nucleic acid molecule in a cell, tissue or organ.

In one embodiment, the promoter may be a constitutive or a strong promoter. Alternatively, the promoter may be a tissue-specific promoter.

A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, maize ubiquitin promoter, rubisco small subunit, maize or alfalfa H3 histone, OCS, SAD1 or 2, GOS2 or any promoter that gives enhanced expression.

A “strong promoter” refers to a promoter that leads to increased or overexpression of the gene. Examples of strong promoters include, but are not limited to, CaMV-35S, CaMV-35Somega, Arabidopsis ubiquitin UBQ1, rice ubiquitin, actin, or Maize alcohol dehydrogenase 1 promoter (Adh-1).

The term “operably linked” as used herein refers to a functional linkage between the promoter sequence and the gene of interest, such that the promoter sequence is able to initiate transcription of the gene of interest.

In one embodiment, the progeny plant is stably transformed with the nucleic acid construct described herein and comprises the exogenous polynucleotide, which is heritably maintained in the plant cell. The method may include steps to verify that the construct is stably integrated. The method may also comprise the additional step of collecting seeds from the selected progeny plant.

In one aspect of the present disclosure, there is provided a genetically altered plant, plant part thereof or plant cell, wherein the plant, part thereof or plant cell comprises at least one mutation in at least one nucleic acid sequence encoding a nucleotide-binding domain and leucine-rich repeat-containing (NLR) protein.

In an alternative aspect of the invention, there is provided a genetically altered plant, plant part thereof or plant cell, wherein the plant, part thereof or plant cell expresses a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain, as described herein.

In one embodiment, the nucleic acid construct expresses a nucleic acid sequence that encodes a NLR protein, wherein the NLR protein is selected from SEQ ID NO: 37, 38, 39, 44, 45, 52, 53 or 54 or a functional variant or homologue therein and wherein preferably the regulatory sequence is operably linked to a regulatory sequence

NLRs belong to the signal ATPases with numerous domains (STAND) superfamily. They typically exhibit a tripartite domain architecture consisting of an N-terminal signaling domain, a central nucleotide binding domain and C-terminal superstructure forming repeats. The central domain, termed NB-ARC (nucleotide binding adaptor shared by APAF-1, plant R proteins and CED-4) or NACHT (shared by NAIP2, C2TA, HET-E and TP1) in plant and animal NLRs, respectively, is a hallmark of this protein family and plays a key role as a molecular switch, mediating conformational changes required for activation. It consists of a nucleotide binding domain (NB), a helical domain (HD1), and a winged-helix domain (WHD) in NB-ARCs, with NACHT domains exhibiting a second helical domain (HD2). Diverse NLR activation and signaling strategies are found in nature. In some cases one NLR protein, termed a singleton, can mediate both elicitor perception and subsequent immune signaling (19). However, some NLRs can function as receptor pairs or in higher order configurations termed immune receptor networks (13, 20). In these cases, one NLR acts as a pathogen sensor, requiring a second “helper” NLR to initiate immune signaling. Such is the case in the solanaceous NRC immune receptor network, which is comprised of multiple sensor NLRs that require an array of downstream helper NLRs termed NRCs (NLRs required for cell death) to successfully initiate immune signaling. The NRC network can encompass up to half of the NLRome in some solanaceous plant species and plays a key role in mediating immunity against a variety of plant pathogens including oomycetes, bacteria, viruses, nematodes and insects.

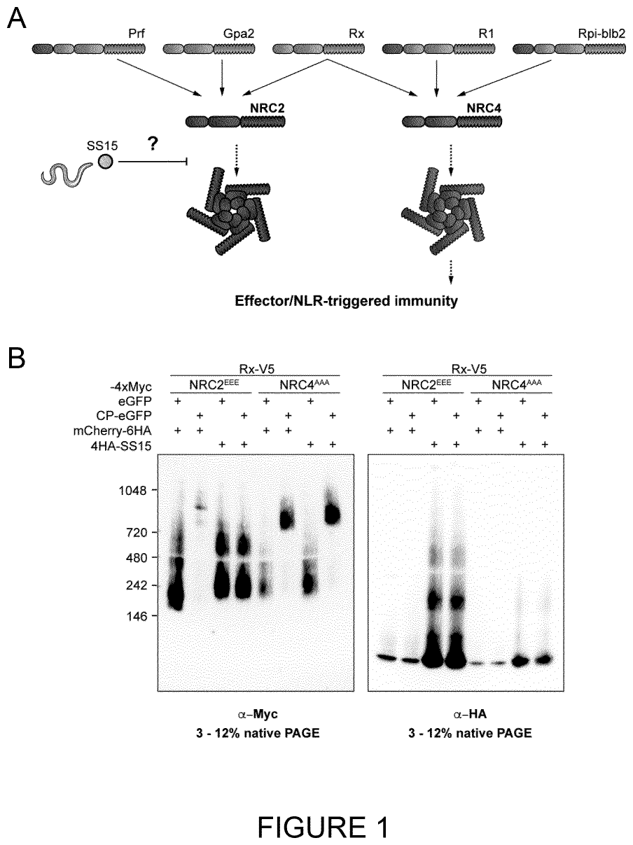

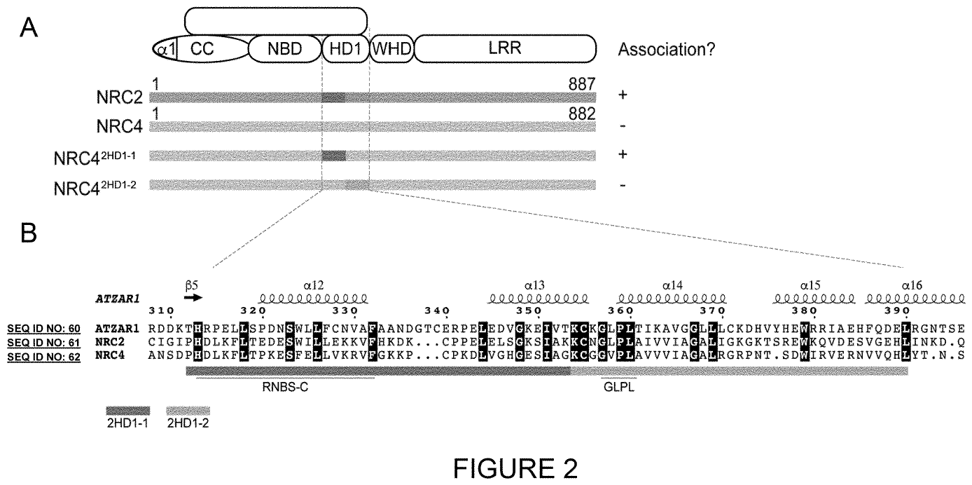

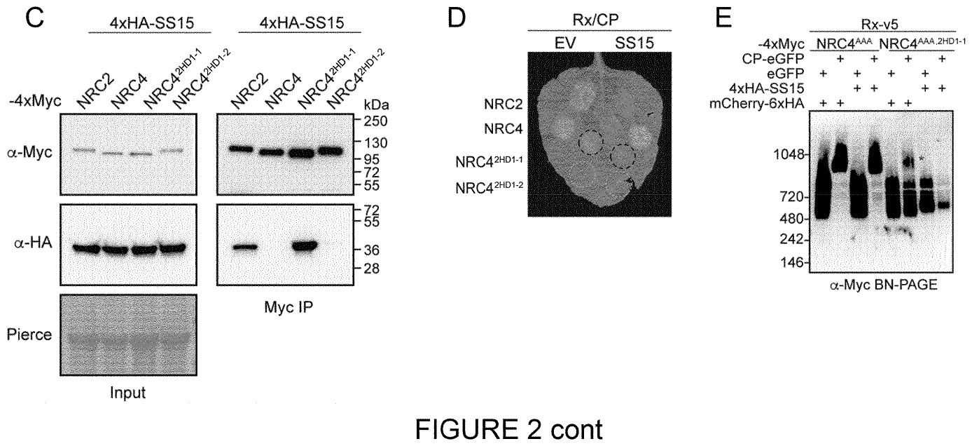

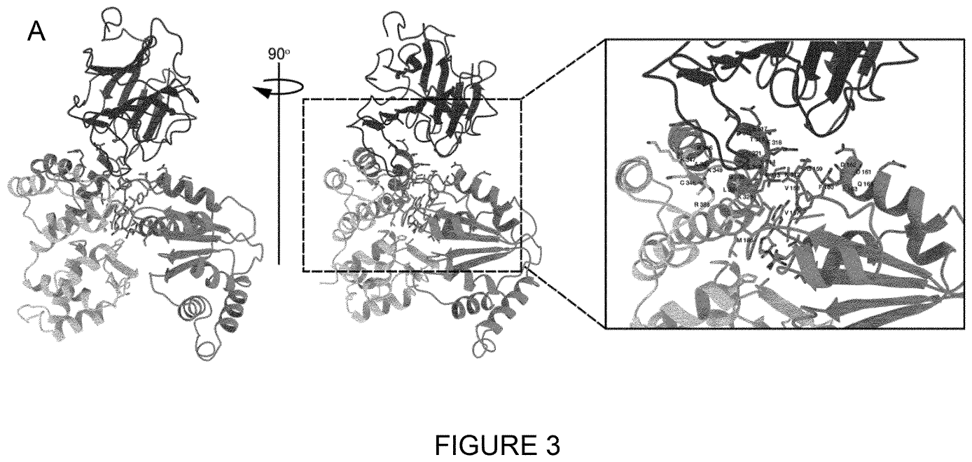

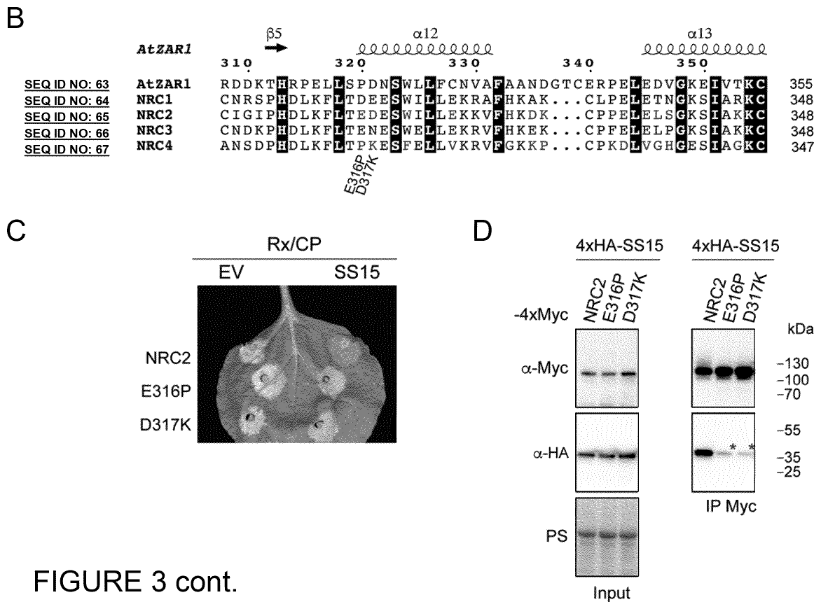

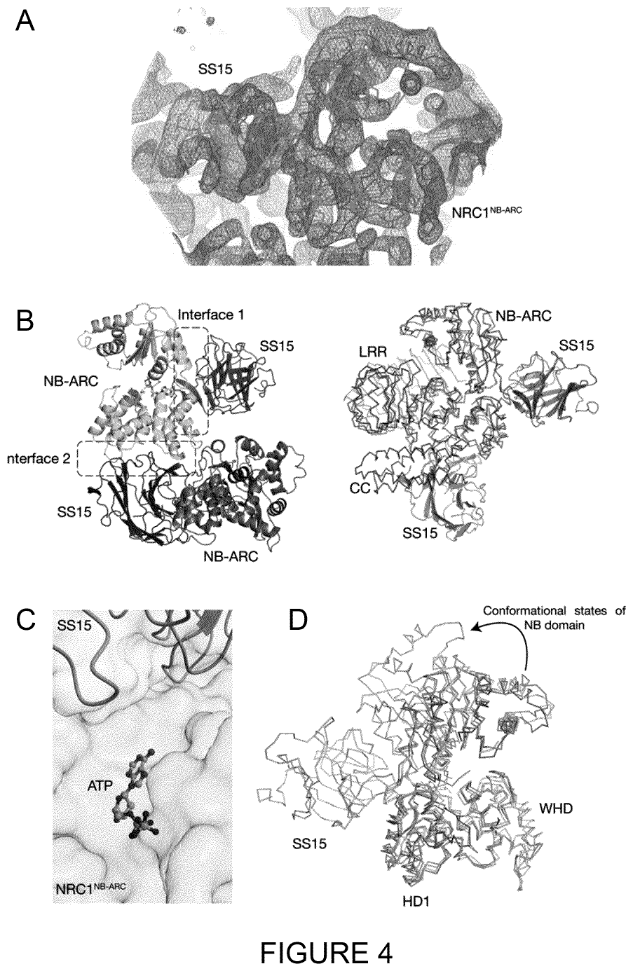

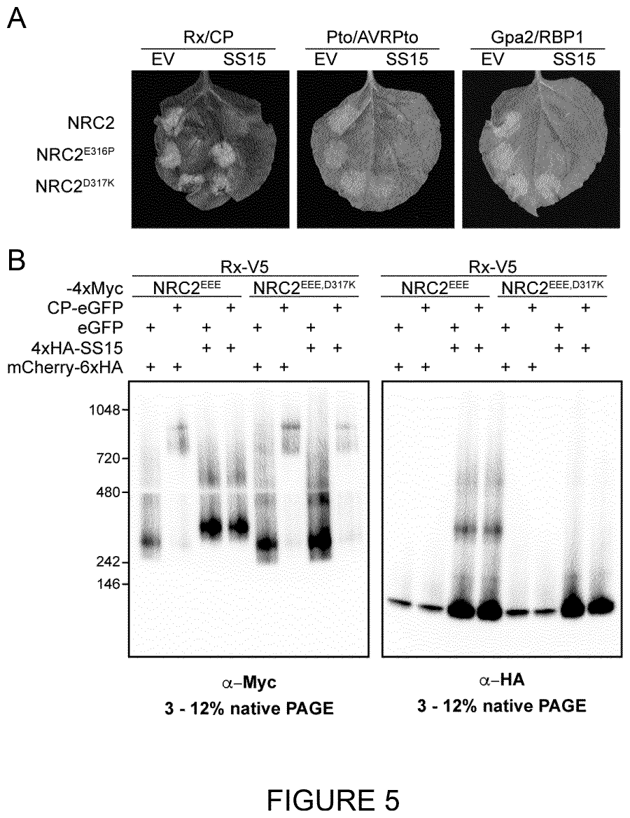

Being engaged in a co-evolutionary arms-race with their hosts, diverse plant and metazoan pathogens have evolved effectors that can interfere with host NLR signaling to promote disease via different strategies. In some cases, effectors can suppress NLR-mediated immunity indirectly by interfering with host proteins that either modulate or act downstream of NLR signaling (15, 17, 21, 22). In other cases, some effectors have evolved to directly interact with NLRs to inhibit their functions (15, 16, 23). One example is the potato cyst nematode effector, SS15, which can suppress signaling mediated by helper NLRs NRC1, NRC2 and NRC3, by directly binding to their central NB-ARC domains (15) (FIG. 1A).

Accordingly, the genetically altered plant, plant part thereof or plant cell, may comprise at least one mutation in at least one nucleic acid sequence encoding a sensor NLR, a singleton NLR, or a helper NLR, such as an NRC protein (for example NRC2 or NRC3). Collectively, singleton NLRs and helper NLRs may be referred to as executor NLRs.

By “at least one mutation in at least one nucleic acid sequence encoding an NLR” is meant that where the NLR gene is present as more than one copy or homeologue (with the same or slightly different sequence) there is at least one mutation in at least one (endogenous) gene. In one embodiment, all genes are mutated. Additionally or alternatively, by “at least one mutation in at least one nucleic acid sequence encoding an NLR” is meant that the nucleic acid sequence of one or more NLR proteins are mutated. For example, just NRC2 (and at least one or all homeologues) is mutated. Alternatively, just NRC3 (and at least one or all homeologues). Or both NRC2 and NRC3 are mutated (and at least one or all homeologues thereof). Preferably, the NLR is not NRC4.

An ‘endogenous’ nucleic acid or gene may refer to the native or natural sequence in the plant genome.

In one embodiment, the NLR may be at least one of NRC1, NRC2 or NRC3.

The nucleic acid sequence of NRC1 may encode an NRC1 protein as defined in SEQ ID NO: 47 or a functional variant or homologue thereof. Preferably, the nucleic acid sequence of NRC2 comprises or consists of SEQ ID NO: 46 or a functional variant or homologue thereof.

The nucleic acid sequence of NRC2 may encode an NRC2 protein as defined in SEQ ID NO: 32, 34, 41 or 49 or a functional variant or homologue thereof. Preferably, the nucleic acid sequence of NRC2 comprises or consists of SEQ ID NO: 31, 33, 40 or 48 or a functional variant or homologue thereof.

The nucleic acid sequence of NRC3 may encode an NRC3 protein as defined in SEQ ID NO: 36, 43 or 51. Preferably, the nucleic acid sequence of NRC3 comprises or consists of SEQ ID NO: 35, 42 or 50 or a functional variant or homologue thereof.

In one embodiment, the NLR protein comprises a HD1 domain, and the at least one mutation is a mutation of one or more nucleotides in the HD1 domain. The HD1 domain may also be referred to as the helical domain or the ARC1 domain. Such terms may be used interchangeably herein.

The HD1 domain is a conserved domain that comprises one or more conserved motifs. Preferably, the HD1 domain comprises a RNBS-C and/or a GLPL motif. In particular however, the HD1 domain may be considered to be a domain of between 50 and 100 amino acids, and more preferably around 60 to 90 amino acids, particularly around 80 amino acids, and that contains a RNBS-C motif.

The RNBS (resistance nucleotide binding site)-C motif may comprise the following consensus sequence: LxxxExWxLF), where the leucines at positions 0 and 8 are highly conserved. In a further embodiment, the RNBS-C motif may comprise an amino acid sequence as defined in SEQ ID NO: 3, 6, 9, 15, 18, 24, 27, or 30 or a functional fragment or variant thereof.

As described with respect to FIG. 2, we have found that only the N-terminal half of the HD1 domain is responsible for binding a pathogen effector, and that in addition, mutations in specifically this region leads to an NLR protein that can evade pathogen effector suppression while retaining complete functionality (e.g. can oligomerize and mediate cell death when activated by effectors or sensor NLRs).

Accordingly, the at least one mutation in an NLR protein is preferably at least one mutation in an N-terminal portion of the HD1 domain. In one example, the N-terminal portion of the HD1 domain containing the one or more mutations may comprise no more than the N-terminal 42 amino acids (e.g. 1 to 42) of the HD1 domain. Preferably, the N-terminal portion of the HD1 domain may comprise no more than the N-terminal 38 amino acids (e.g. amino acids 1 to 38) of the HD1 domain. Preferably the N-terminal portion of the HD1 domain comprises or consists of the RNBS-C motif, as described above. The N-terminal portion of HD1 may also be referred to as HD1-1 herein.

The HD1-1 region may comprise or consist of a nucleic acid sequence that encodes an amino acid sequence as defined in SEQ ID NO: 2, 5, 8, 14, 17, 23, 26 or 29 or a functional variant or fragment thereof. Preferably, the functional variant or fragment comprises at least a RNBC-C motif as described above. Preferably, the at least one mutation is in the RNBC-C motif.

The term “variant” or “functional variant” as used herein with reference to any of the sequences defined herein refers to a variant gene sequence or part of the gene sequence which retains the biological function of the full non-variant sequence. Accordingly, in the context of an NLR protein, a functional variant may be a variant that is able to mediate an immune response, such as the hypersensitive (HR) response. For an NRC protein, a functional variant may be able to oligomerise to initiate the immune response. In the context of HD1 or HD1-1, a functional variant may be a variant that facilitates the oligomerisation of the NRC protein to initiate the immune response.

A functional variant also comprises a variant of the gene of interest, which has sequence alterations that do not affect function, for example in non-conserved residues. Also encompassed is a variant that is substantially identical, i.e. has only some sequence variations, for example in non-conserved residues, compared to the wild type sequences as shown herein and is biologically active (e.g. is able to oligomerise and cause cell death). Alterations in a nucleic acid sequence that result in the production of a different amino acid at a given site that does not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

As used in any aspect of the invention described herein a “variant” or a “functional variant” has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates an NLR gene orthologue from other plant species. Suitable homologues can be identified by sequence comparisons and identifications of conserved domains as described above. There are predictors in the art that can be used to identify such sequences. The function of the homologue can be identified as described herein and a skilled person would thus be able to confirm the function, for example when overexpressed in a plant.

A homolog may also have, in increasing order of preference, at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid sequences referenced herein or to the nucleic acid sequences referenced herein. Functional variants of NLR gene homologs as defined above are also within the scope of the invention.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognised that residue positions that are not identical often differ by conservative amino acid substitutions, where amino acids residues are substituted for other amino acid residues with similar chemical properties (e.g., charge or hydrophobicity) and therefore do not change the functional properties of the molecule.

Where sequences differ in conservative substitutions, the percent sequence identity may be adjusted upwards to correct for the conservative nature of the substitution. Means for making this adjustment are well known to those of skill in the art. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Non-limiting examples of algorithms that are suitable for determining percent sequence identity and sequence similarity are the BLAST and BLAST 2.0 algorithms. The overall sequence identity of a variant can be determined using any number of sequence alignment programs known in the art. As an example, Emboss Stretcher from the EMBL-EBI may be used: https://www.ebi.ac.uk/Tools/psa/emboss_stretcher/(using default parameters: pair output format, Matrix=BLOSUM62, Gap open=1, Gap extend=1 for proteins; pair output format, Matrix=DNAfull, Gap open=16, Gap extend=4 for nucleotides).

The skilled person would understand that suitable homologues and the homologous positions in these sequences can be identified by sequence comparisons (e.g. BLAST, alignments) and identifications of conserved domains. Phylogenetic tree analysis using nucleotide or amino acid sequences can be used to establish orthology to an NLR gene. There are predictors in the art that can be used to identify such sequences. The function of the homologue can be identified as described herein and a skilled person would thus be able to confirm the function, for example using a programmed cell death assay. Homologous positions or as used herein “corresponding positions in homologous sequences” can thus be determined by performing sequence alignments once the homologous sequence has been identified. For example, homologues can be identified using a BLAST search of the plant genome of interest using the S. tuberosum or N. benthamiana NRC2 or NRC3 sequence as a query (i.e. one of the sequences defined in SEQ ID Nos: 31 to 36 or 40 to 43).

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domains structure (e.g. presence of a RNBS-C motif) can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, New York).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Duration of hybridization is generally less than 24 hours, usually about 4 to 12. Stringent conditions may also be achieved with the addition of destabilizing agents such as formamide.

In a further embodiment, a variant as used herein can comprise a nucleic acid sequence encoding an NLR polypeptide as defined herein that is capable of hybridising under stringent conditions as defined herein to a nucleic acid sequence as defined herein.

In one embodiment, there is provided a genetically altered plant, plant part thereof or plant cell, wherein the plant, part thereof or plant cell comprises at least one mutation in at least one nucleic acid sequence encoding at least one NLR protein, preferably NRC1, NRC2 and/or NRC3,
wherein the NRC1 gene comprises or consists of
   a. a nucleic acid sequence encoding a polypeptide as defined in one of SEQ ID NOs: 47; or
   b. a nucleic acid sequence as defined in one of SEQ ID NOs: 46 or
   c. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
   d. a nucleic acid sequence encoding a NRC2 polypeptide as defined herein that is capable of hybridising under stringent conditions as defined herein to the nucleic acid sequence of any of (a) to (c); and wherein the NRC2 gene comprises or consists of
   e. a nucleic acid sequence encoding a polypeptide as defined in one of SEQ ID NOs: 32, 34, 41 or 49; or
   f. a nucleic acid sequence as defined in one of SEQ ID NOs: 31, 33, 40 or 48 or
   g. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (e) or (f); or
   h. a nucleic acid sequence encoding a NRC2 polypeptide as defined herein that is capable of hybridising under stringent conditions as defined herein to the nucleic acid sequence of any of (e) to (g); and
wherein the NRC3 gene comprises or consists of
   i. a nucleic acid sequence encoding a polypeptide as defined in one of SEQ ID NOs: 36, 43 or 51; or
   j. a nucleic acid sequence as defined in one of SEQ ID NOs: 35, 42 or 50 or
   k. a nucleic acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to either (a) or (b); or
   l. a nucleic acid sequence encoding a NRC3 polypeptide as defined herein that is capable of hybridising under stringent conditions as defined herein to the nucleic acid sequence of any of (a) to (c).

Preferably, the at least one mutation that is introduced into at least one NLR protein, and specifically, the HD1-1 region of an NLR protein, leads to an alteration or mutation of the corresponding amino acid sequence of the NLR protein. As shown in FIG. 5, where the NLR protein is an NRC protein, the at least one mutation preferably reduces or prevents the inhibition by a pathogen effector of oligomerization of the NRC protein into a complex for initiating an immune response. That is, the pathogen effector is no longer able to suppress the function (immune activating) of the NRC protein. The oligomerization of a NRC protein can be measured by, for example, BN-PAGE.

More preferably, the at least one mutation reduces or prevents binding between the NLR protein and the pathogen effector. Binding between the NLR protein and the pathogen effector can be determined by any routine methods in the art, such as co-immunoprecipitation, as described in the Examples.

Importantly, the mutation does not affect the function of the NLR protein. In one example, the at least one mutation reduces or prevents the inhibition by a pathogen effector of the initiation of said immune response pathway by the NLR protein. In the case of an NRC protein, the NRC protein is (still) able to oligomerise and/or mediate an immune response, such as a cell death response. In one example, the mutation preserves (or does not alter) the downstream interactions/signalling pathway of the native NLR protein. For example, where the native NLR protein initiates an immune response via one or more interactions with one or more downstream molecules, the mutated NLR protein initiates an immune response via the same interactions.

As shown in FIG. 3 and FIG. 5, we have also shown that mutation of the HD1-1 region in particular, maintains upstream signalling with sensor NLRs, evades suppression by the pathogen effector, and compromises pathogen effector binding to the NLR (i.e. has reduced or no binding to the NLR).

The at least one mutation that is introduced into at least one at least one nucleic acid sequence encoding at least one NRC protein can be selected from the following mutation types:
1. a “missense mutation”, which is a change in the nucleic acid sequence that results in the substitution of one amino acid for another amino acid;
2. a “nonsense mutation” or “STOP codon mutation”, which is a change in the nucleic acid sequence that results in the introduction of a premature STOP codon and, thus, the termination of translation (resulting in a truncated protein); in plants, the translation stop codons may be selected from “TGA” (UGA in RNA), “TAA” (UAA in RNA) and “TAG” (UAG in RNA); thus any nucleotide substitution, insertion, deletion which results in one of these codons to be in the mature mRNA being translated (in the reading frame) will terminate translation;
3. an “insertion mutation” of one or more nucleotides or one or more amino acids, due to one or more codons having been added in the coding sequence of the nucleic acid;
4. a “deletion mutation” of one or more nucleotides or of one or more amino acids, due to one or more codons having been deleted in the coding sequence of the nucleic acid;
5. a “frameshift mutation”, resulting in the nucleic acid sequence being translated in a different frame downstream of the mutation. A frameshift mutation can have various causes, such as the insertion, deletion or duplication of one or more nucleotides;
6. a “splice site” mutation, which is a mutation that results in the insertion, deletion or substitution of a nucleotide at the site of splicing (i.e. either a splice acceptor or splice donor mutation),
   wherein preferably, any one or more of the above mutations leads to reduced or abolished binding between the NLR protein and a pathogen effector. As described above, by “binding” is meant a directly or indirect association with each other. That is, the NLR and the pathogen effector co-immunoprecipitate when expressed in planta. The reduction in binding between a mutated NLR and a pathogen effector may be at least 10%, at least 20%, at least 30%, at least 40%, at least 50%, at least 60%, at least 70%, at least 80%, at least 90%, at least 95% compared to the amount of binding (measurable, for example, by co-ip) between the wild-type NLR and the pathogen effector. Alternatively, no interaction between the NLR and the pathogen effector can be detected—that is, binding is abolished.

Preferably, the one or more mutation is a substitution mutation. For example, the mutation may be any substitution in HD1-1 or, more preferably, any substitution in the RNBS-C motif of HD1-1.

As shown in FIG. 3, the substitution one or more positions in the amino acid sequence, where the position is selected from 315, 316 or 317 of any of SEQ ID NO: 32, 34, 36, 41, 47, 49 or 51 or a homolog or functional variant thereof. Alternatively, the substitution is a homologous position in a homologous sequence.

Preferably, the substitution is a substitution to a hydrophilic and/or positively charged amino acid. In one embodiment, the substitution is a substitution with an amino acid of opposite biochemical property—for example, but not limited to, Lysine to Aspartic Acid (Positive to Negative charge), Serine to Alanine (Polar to non-polar).

In one embodiment, the mutation is a substitution of a D residue. Alternatively, the mutation is a substitution of a E residue. Alternatively, the mutation is a substitution of a N residue. Alternatively, the mutation is a substitution of a S residue.

In one embodiment, the substitution is a D317K substitution or a homologous position in a homologous sequence in SEQ ID NO: 32 or 34 or a homologue or functional variant thereof.

In one embodiment, the substitution is a N317K substitution or a homologous position in a homologous sequence in SEQ ID NO: 36 or a homologue or functional variant thereof.

In one embodiment, the substitution is a D315K substitution or a homologous position in a homologous sequence in SEQ ID NO: 41, 43, 49 or 51 or a homologue or functional variant thereof.

In one embodiment, the substitution is a D316K substitution or a homologous position in a homologous sequence in SEQ ID NO: 47, or a homologue or functional variant thereof.

In one embodiment, the substitution is a S317K substitution or a homologous position in a homologous sequence in SEQ ID NO: 41, 43, 49 or 51 or a homologue or functional variant thereof.

In one embodiment, the substitution is a E317K substitution or a homologous position in a homologous sequence in SEQ ID NO: 47 or a homologue or functional variant thereof.

Preferably, as shown in FIG. 5, the substitution is preferably at position D317 of SEQ ID NO: 32 or 34 or a homologous position in a homologous sequence, wherein preferably the substitution is D317K.

Homologous positions or as used herein “corresponding positions in homologous sequences” can be determined by performing sequence alignments once the homologous sequence has been identified. For example, homologues can be identified using a BLAST search of the plant genome of interest using the S. tuberosum or N. benthamiana NRC1, NRC2 or NRC3 sequence as a query (i.e. one of the sequences defined in SEQ ID NOs: SEQ ID Nos: 32, 34, 36, 41, 43, 47, 49 or 51).

Alternatively, the mutation is a substitution of all or a significant proportion of the HD1-1 region for a corresponding HD1-1 region in a second NLR protein, wherein the second NLR protein is not inhibited by the pathogen effector.

The mutation may also be a substitution of all or a significant proportion of the RNBS-C motif for a corresponding RNBS-C motif in a second NLR protein, wherein the second NLR protein is not inhibited by the pathogen effector.

By “not inhibited” is meant that either the pathogen effector cannot bind or significantly bind to the second NLR protein, or that the pathogen effector does not inhibit or prevent the second NLR protein from eliciting an immune response, such as cell death. In one example, the second NLR protein may be NRC4.

The nucleic acid sequence of the NRC4 RNBS-C motif may encode a RNBS-C motif as defined in SEQ ID NO: 12 or a homologue or functional variant thereof.

In one embodiment, the mutation is introduced using targeted genome editing. That is, in one embodiment, the invention relates to a method and plant that has been generated by genetic engineering methods as described above, and does not encompass naturally occurring varieties or generating plants by traditional breeding methods.

Targeted genome modification or targeted genome editing is a genome engineering technique that uses targeted DNA double-strand breaks (DSBs) to stimulate genome editing through homologous recombination (HR)-mediated recombination events.

In a preferred embodiment, the genome editing method that is used according to the various aspects of the invention is CRISPR. Alternatively, targeted genome editing can be performed using TALENs.

In a preferred embodiment of any aspect of the invention described herein, the sgRNA can be used with a modified Cas9 protein, such as nickase Cas9 or nCas9 or a “dead” Cas9 (dCas9) fused to a “Base Editor”-such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made (Kim et al., 2017; Gaudelli et al. 2017). Alternatively, the method may use sgRNA together with a template or donor DNA constructs, to introduce a targeted SNP or mutation, in particular one of the substitutions described herein, into a NRC gene. In this embodiment, the introduction of a template DNA strand, following a sgRNA-mediated snip in the double-stranded DNA, can be used to produce a specific targeted mutation (i.e. a SNP) in the gene using homology directed repair. As a further alternative, prime editing can be used to introduce the specific mutation (Anzalone et al., 2019). Here a catalytically impaired Cas9 endonuclease is fused to an engineered reverse transcriptase programmed with a prime editing guide RNA (pegRNA) that is both specific to the target site and encodes the desired edit. Using the above methods we can mutate the nucleotides that encode the conserved HD1-1 domain and therefore lead to one of the above-described substitutions in the amino acid sequence.

Once targeted genome editing has been performed, rapid high-throughput screening procedures can be used to analyse amplification products for the presence of a mutation in the NLR gene, and in particular, in the HD1-1 domain. Once a mutation is identified, and screened for the increased immunity to a pathogen effector, stable, non-segregating plant lines are produced. Mutants with one or more mutations in an NLR gene, and in particular in the HD1-1 region, and as a result, increased immunity to a given pathogen compared to a control plant can thus be identified.

In one embodiment, one targeted genome editing has been performed, the mutant in NLR protein is transiently expressed in N. Benthamiana to confirm that the mutant is suppression insensitive (i.e. resurrected). This may be determined using a programmed cell death assay, either activating it with auto active mutations or activating it with sensor+avirulence effector protein. In a further embodiment, a proof of concept experiment is carried out in a crop plant, such as soybean.

Plants obtained or obtainable and seeds or other reproductive material obtained or obtainable from such plants by such method which carry a functional mutation in at least one endogenous NLR gene (preferably NRC2 and/or NRC3) are also within the scope of the invention.

In one embodiment, the progeny plant is stably transformed with the CRISPR constructs, and comprises the exogenous polynucleotide which is heritably maintained in the plant cell. The method may include steps to verify that the construct is stably integrated. The method may also comprise the additional step of collecting seeds or other reproductive material from the selected progeny plant.

In one example, the plant may not comprise a mutation introduced into any other gene. In other words, only a mutation is introduced into one or more NLR proteins as described above.

In another aspect of the invention, there is provided a method of providing or improving pathogen resistance in a plant, the method comprising introducing at least one of the above-described mutations into at least one nucleic acid sequence encoding a nucleotide-binding domain and leucine-rich repeat-containing (NLR) protein, wherein the NLR protein comprises a HD1 domain; and wherein the at least one mutation is a mutation of one or more nucleotides in an N-terminal portion of the HD1 domain, as described above.

In another aspect of the invention, there is provided a method of providing or improving pathogen resistance in a plant, the method comprising introducing and expressing a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

In another aspect of the invention, there is also provided a method of producing a plant with improved pathogen resistance, the method comprising introducing at least one of the above-described mutations into at least one nucleic acid sequence encoding a nucleotide-binding domain and leucine-rich repeat-containing (NLR) protein, wherein the NLR protein comprises a HD1 domain; and wherein the at least one mutation is a mutation of one or more nucleotides in an N-terminal portion of the HD1 domain, as described above.

In another aspect of the invention, there is also provided a method of producing a plant with improved pathogen resistance, the method comprising introducing and expressing a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

By “providing or improving pathogen resistance” is meant a reduction in at least one of pathogen/pest growth, fitness or an improvement of the fecundity on the plant carrying a mutated NLR as described above, compared to a wild-type or control plant. A reduction or improvement as used herein may be may be at least 10%, at least 20%, at least 30%, at least 40%, at least 50%, at least 60%, at least 70%, at least 80%, at least 90% or at least 95% compared to the level of pathogen/pest growth or fitness in a control or wild-type plant or compared to the fecundity of a control or wild-type plant.

A “pathogen” may include any disease causing agent, particularly a disease causing organism such as bacteria, viruses, nematodes and fungi. Pathogens infect plants and cause disease, and may, for example, reduce yield or otherwise damage the plant, for example through the action of toxins. “Pests” may include organisms such as animals, insects or nematodes, and may infect plants with pathogens or cause damage to the plant directly (e.g. through sap-feeding, boring into stems and fruits, or and cutting the root stem and leaves).

Alternatively, the provision of or an improvement in pathogen resistance can lead to an increase in yield or seed yield. As such, the provision of or an improvement in pathogen resistance can be measured by measuring an increase in yield or seed yield.

The term “yield” in general means a measurable produce of economic value, typically related to a specified crop, to an area, and to a period of time. Individual plant parts directly contribute to yield based on their number, size and/or weight. The actual yield is the yield per square meter for a crop and year, which is determined by dividing total production (includes both harvested and appraised production) by planted square metres.

Preferably, increased yield comprises at least one of an increased number and/or weight of seeds, increased number of pods per plant (where the plant contains pods), increased thousand kernel weight (TKW), increased biomass, increased fresh weight and increased growth, preferably root growth. Yield is increased relative to a control or wild-type plant. For example, the yield is increased by may be at least 10%, at least 20%, at least 30%, at least 40%, at least 50%, at least 60%, at least 70%, at least 80%, at least 90%, at least 95% compared to a wild-type or control plant.

In another aspect of the invention, there is provided a method of improving plant immunity, the method comprising introducing at least one of the above-described mutations into at least one nucleic acid sequence encoding a nucleotide-binding domain and leucine-rich repeat-containing (NLR) protein, wherein the NLR protein comprises a HD1 domain; and wherein the at least one mutation is a mutation of one or more nucleotides in an N-terminal portion of the HD1 domain, as described above.

In another aspect of the invention, there is provided a method of improving plant immunity, wherein the method comprises introducing and expressing a nucleic acid construct comprising a nucleic acid sequence encoding a mutated NLR protein, as described herein, wherein the NLR protein comprises a HD1 domain, and wherein the NLR protein comprises a mutation of one or more amino acids in an N-terminal portion of the HD1 domain.

In one embodiment, the nucleic acid construct expresses a nucleic acid sequence that encodes a NLR protein, wherein the NLR protein is selected from SEQ ID NO: 37, 38, 39, 44, 45, 52, 53, 54, 55, 56, 57, 58 or 59 or a functional variant or homologue therein and wherein preferably the regulatory sequence is operably linked to a regulatory sequence

In another aspect of the invention, there is also provided a method of producing a plant with improved immunity, the method comprising introducing at least one of the above-described mutations into at least one nucleic acid sequence encoding a nucleotide-binding domain and leucine-rich repeat-containing (NLR) protein, wherein the NLR protein comprises a HD1 domain; and wherein the at least one mutation is a mutation of one or more nucleotides in an N-terminal portion of the HD1 domain, as described above.

In another aspect of the invention, there is also provided a method of producing a plant with improved immunity, wherein the method comprises introducing and expressing a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain, as described herein.

In one embodiment, the nucleic acid construct expresses a nucleic acid sequence that encodes a NLR protein, wherein the NLR protein is selected from SEQ ID NO: 37, 38, 39, 44, 45, 52, 53, 54, 55, 56, 57, 58 or 59 or a functional variant or homologue therein and wherein preferably the regulatory sequence is operably linked to a regulatory sequence

An improvement in plant immunity can be measured by any technique in the art. In one embodiment, an improvement in immunity can be measured by measuring cell death in the presence of a pathogen or pathogen effector compared to cell death in a wild-type or control plant. An example of a cell death assay that may be carried out is described in the Examples.

By “improvement” is meant an improvement of at least 10%, at least 20%, at least 30%, at least 40%, at least 50%, at least 60%, at least 70%, at least 80%, at least 90%, at least 95% in immunity compared to a wild-type or control plant.

In another aspect of the invention, there is provided a method of producing an altered NLR protein, wherein a pathogen effector is either unable or has reduced ability to bind to the NLR protein. “Binding” is defined above. Preferably, the method comprises introducing at least one of the above-described mutations into an N-terminal portion of an NLR protein, as described above.

The method may comprise
a. selecting a part of the plant;
b. transforming at least one cell of the part of the plant of paragraph (a) with at least one CRISPR construct or sgRNA molecule, wherein the CRISPR construct or sgRNA molecule targets the NRC gene, and introduces at least one mutation into the HD1-1 region as described above;
c. regenerating at least one plant derived from the transfected cell or cells;
d. selecting one or more plants obtained according to paragraph (c) that show at least one mutation in the HD1-1 region.

In one embodiment, the method may comprise obtaining a DNA sample from a transformed plant and carrying out DNA amplification to detect the at least one mutation in the HD1-1 region. In a further embodiment of any of the methods described herein, the method may further comprise at least one or more of the steps of assessing the phenotype of the genetically altered plant and measuring at least one of immunity to a given pathogen In other words, the method may involve the step of screening the plants for the desired phenotype.

Alternatively, more conventional mutagenesis methods can be used to introduce at least one mutation into the HD1-1 region of an NLR gene. These methods include both physical and chemical mutagenesis. A skilled person will appreciate that further approaches can be used to generate such mutants, and methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein.

In another embodiment, mutagenesis is physical mutagenesis, such as application of ultraviolet radiation, X-rays, gamma rays, fast or thermal neutrons or protons. The targeted population can then be screened to identify a substitution mutation in HD1-1 region of an NLR gene.

In another embodiment of the various aspects of the invention, the method comprises mutagenizing a plant population with a mutagen. The mutagen may be a fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz (a) anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9 [3-(ethyl-2-chloroethyl)aminopropylamino]acridine dihydrochloride (ICR-170) or formaldehyde. Again, the targeted population can then be screened to identify one of the above described mutations in the HD1-1 region of an NLR gene.

In another embodiment, the method used to create and analyse mutations is targeting induced local lesions in genomes (TILLING). In an alternative embodiment, the method used to create and analyse mutations is EcoTILLING. EcoTILLING is molecular technique that is similar to TILLING, except that its objective is to uncover natural variation in a given population as opposed to induced mutations. The first publication of the EcoTILLING method was described in Comai et al. 2004.

In another embodiment, the method uses oligonucleotide directed mutagenesis (ODM).

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that is not transformed or genetically altered as described herein.

In a further aspect of the invention there is provided a plant obtained or obtainable by the above-described methods. In a further aspect, there is provided a seed or other reproductive material obtained or obtainable from the plant. Also included in the scope of the invention is progeny plants obtained from the seed or other reproductive material and as well as seed or other reproductive material obtained from the progeny plants.

In another aspect of the invention, there is provided a method of screening a population of plants and identifying and/or selecting a plant that will exhibit pathogen resistance or improved pathogen resistance, the method comprising detecting in the plant or plant germplasm at least one polymorphism (or mutation, as described above) in a NRC2 and/or NRC3 gene, wherein preferably the polymorphism is in an N-terminal portion of the HD1 domain of the NRC2 and/or NRC3 gene; and selecting said plant.

Suitable tests for assessing the presence of a polymorphism would be well known to the skilled person, and include but are not limited to DNA sequencing (e.g. amplicon sequencing, RenSeq (resistance gene enrichment sequencing), or Sanger sequencing with a primer), Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In one embodiment, the method comprises
a) obtaining a nucleic acid sample from a plant and
b) carrying out nucleic acid amplification of NRC2 and/or NRC3 alleles using one or more primer pairs.

In a further embodiment, the method may further comprise introgressing or crossbreeding the chromosomal region comprising at least one of said NRC polymorphisms as described above into a second plant or plant germplasm to produce an introgressed plant or plant germplasm.

A “pathogen effector” as described herein may refer to any protein originating from the pathogen that operates in the host (plant) for the benefit of the pathogen. In particular, this includes any pathogen effector that is capable of binding to an NLR protein, and for example to the HD1-1 region of NRC2 and/or NRC3. Examples of such pathogen effectors include, SPRYSEC10 (SS10), SPRYSEC34 (SS34), SPRYSEC15 (SS15) from the potato cyst nematode pathogen, Globodera rostochiensis. Other examples include AVRcap1b and PITG-15278, which are RXLR-WY/LWY domain containing effectors from Phytophthora infestans. Preferably, the pathogen effector is SS15. Preferably the pathogen is a potato cyst nematode.

In one embodiment, the plant is a crop plant. By crop plant is meant any plant which is grown on a commercial scale for human or animal consumption or use. In another embodiment the plant is Arabidopsis, Nicotiana benthamiana, Nicotiana tabacum, Medicago truncatula, or other suitable model organisms used in plant research.

The plant may be a dicot or a monocot.

A dicot plant may be selected from the families including, but not limited to Asteraceae, Brassicaceae (eg Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species. In one embodiment, the plant is oilseed rape.

Also included are biofuel and bioenergy crops such as rape/canola, sugar cane, sweet sorghum, Panicum virgatum (switchgrass), linseed, lupin and willow, poplar, poplar hybrids, Miscanthus or gymnosperms, such as loblolly pine. Also included are crops for silage (maize), grazing or fodder (grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed) and for amenity purposes (e.g. turf grasses for golf courses), ornamentals for public and private gardens (e.g. snapdragon, petunia, roses, geranium, Nicotiana sp.) and plants and cut flowers for the home (African violets, Begonias, chrysanthemums, geraniums, Coleus spider plants, Dracaena, rubber plant).

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

Preferably, the plant is a crop plant. By crop plant is meant any plant which is grown on a commercial scale for human or animal consumption or use. Preferred plants are maize, wheat, rice, oilseed rape, sorghum, soybean, potato, tomato, grape, barley, pea, bean, field bean, lettuce, cotton, sugar cane, sugar beet, broccoli or other vegetable brassicas or poplar.

In a preferred embodiment, the plant is selected from the Solanaceae family. In one example, the plant may be selected from potato (Solanum tuberosum), eggplant (Solanum melongena), petunia (Petunia spp., e.g., Petunia x hybrida or Petunia hybrida), tomatillo (Physalis philadelphica), Cape gooseberry (Physalis peruviana), Physalis sp., woody nightshade (Solanum dulcamara), garden huckleberry (Solanum scabrum), gboma eggplant (Solanum macrocarpon), pepper (Capsicum spp.; e.g., Capsicum annuum, C. baccatum, C. chinense, C. frutescens, C. pubescens), tomato (Solanum lycopersicum or Lycopersicon esculentum), tobacco {Nicotiana spp., e.g. N. tabacum, N. benthamiana), Solanum americanum, Solanum demissum, Solanum stoloniferum, Solanum papita, Solanum bulbocastanum, Solanum edinense, Solanum schenckii, Solanum hjertingii, Solanum venturi, Solanum mochiquense, Solanum chacoense, and Solanum pimpinelli folium. Preferred solanaceous plants are solanaceous plants grown in agriculture including, but not limited to, potato, tomato, tomatillo, eggplant, pepper, tobacco, Cape gooseberry, and petunia.

The term “plant” as used herein encompasses whole plants and progeny of the plants and plant parts, including seeds, fruit, shoots, stems, leaves, roots (including tubers), flowers, tissues and organs, wherein each of the aforementioned mutations of the invention. The term “plant” also encompasses plant cells, suspension cultures, callus tissue, embryos, meristematic regions, gametophytes, sporophytes, pollen and microspores. The invention also extends to harvestable parts of a plant of the invention as described herein, but not limited to seeds, leaves, fruits, flowers, stems, roots, rhizomes, tubers and bulbs.

The term “reproductive material” encompasses seeds and other vegetative propagation material, such as tubers.

In a most preferred embodiment, the plant part or harvestable product is a seed or grain. Therefore, in a further aspect of the invention, there is provided a seed or grain produced from a genetically altered plant as described herein. Accordingly, in one aspect of the invention there is provided seed, wherein the seed comprises at least one of the above-described mutations in an NLR gene. Also provided is a progeny plant obtained from the seed as well as seed obtained from that progeny.

A control plant as used herein according to all of the aspects of the invention is a plant, which has not been modified according to the methods of the invention. Accordingly, in one embodiment the control plant does not have one or more mutations in an NLR gene as described herein. In one embodiment, the control plant is a wild type plant. The control plant is typically of the same plant species, preferably having the same genetic background as the modified plant. The control plant may also be the wild-type plant harbouring the same changes as the invention, minus the specific changes that are believed to provide the phenotype. e.g. a control plant comprises another copy of the WT gene, not the edited gene.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

Unless context dictates otherwise, the descriptions and definitions of the features set out above are not limited to any particular aspect or embodiment of the invention and apply equally to all aspects and embodiments, which are described.

The invention is now described in the following non-limiting examples.

### EXAMPLES

Pathogens suppress NLRs to counteract immunity. Here, we show that a cyst nematode effector inhibits resistosome formation of a helper NLR immune protein, NRC2, via direct binding, which in turn physically prevents intramolecular rearrangements required for activation. This results in suppression of immune signaling and disease resistance. By combining structural approaches and leveraging NLR diversity, we engineered a single amino acid point mutant of the helper NLR that evades parasite suppression. This engineered helper NLR resurrected the activity of a cyst nematode disease resistance protein. This represents a novel strategy for editing immune receptor genes to resurrect cryptic/defeated disease resistance in crop genomes.

We provide an approach to rationally design NLRs that evade suppression by pathogens. Relieving pathogen suppression of helper NLRs such as NRC2 allows for the “resurrection” of cryptic upstream sensors that are normally ineffective due to interference with their downstream signaling partners.

### SS15 Directly Inhibits NRC2 Resistosome Formation

NRC2 or NRC4 were transiently expressed with their upstream sensor Rx and the effector SS15 in leaves of nrc2/3/4 CRISPR KO Nicotiana benthamiana plants. BN-PAGE-based readouts were leveraged for NRC resistosome formation. For biochemical analyses, we used NRC2 and NRC4 variants with mutations in their N-terminal MADA motifs (NRC2^{EEE} and NRC4^{AAA}, respectively) which abolish cell death induction without compromising receptor activation, oligomerization or localization. We then activated the Rx-NRC system by co-expressing PVX CP (potato virus X coat protein)-GFP or co-expressed free GFP as an inactive control. In the absence of SS15, both NRC2 and NRC4 oligomerize upon effector-triggered activation mediated by their upstream sensor. However, when SS15 is present, Rx/CP-activated NRC2 is unable to oligomerize and appears as a band of ~240 kDa, which co-migrates with SS15. Inactive NRC2 co-expressed with SS15 also migrates as a band of ~240 kDa, which is slower-migrating relative to inactive NRC2 in the absence of SS15, indicative of in vivo NRC2-SS15 complex formation (FIG. 1B). We also observed that SS15 co-expression not only blocks NRC2 oligomerization but also prevents the previously reported shift of activated NRC2 from cytoplasm to plasma membrane (PM) as well as the formation of NRC2 PM-associated puncta upon Rx/CP activation. We conclude that SS15 can suppress immune signaling by acting as a direct proteinaceous inhibitor of NRC2, by directly binding to its NB-ARC domain in order to block the formation of a signal-competent oligomeric resistosome.

### The HD1-1 Region of the NB-ARC Domain Determines SS15 Association and Inhibition of NRCs

We generated a series of NRC2-NRC4 chimeric proteins in the NB-ARC domain to which SS15 binds (FIG. 2A-2B), which we subsequently assayed for gain or loss of SS15 association via in planta co-immunoprecipitation. We identified one chimeric variant of NRC4, NRC42HD1-1, carrying an N-terminal portion of the HD1 region of NRC2 (referred to herein as the “HD1-1” region), which gains association to SS15 (FIG. 2C). NRC42HD1-1 is susceptible to inhibition by SS15 and is unable to oligomerize and trigger cell death in the presence of SS15 (FIG. 2D, dashed black circles, FIG. 2E). We conclude that SS15 binds to the HD1-1 region and that binding to the HD1-1 is sufficient for this effector to act as a direct inhibitor of NRC resistosome formation and programmed cell death.

### Identification of SS15-NRC Binding Interface Enables Engineering of NRC2 to Evade Pathogen Suppression

We hypothesized that if we identified key residues mediating interaction with the HD1-1 region, we could mutate them to make NRC2 evade immune suppression by SS15. To this end, we produced crystals of SS15 in complex with the NB-ARC domain of the NRC2 homolog, NRC1, which is also suppressed by SS15, and obtained X-ray diffraction data which we solved to 4 Å resolution (FIG. 3A, FIG. 4). We determined that SS15 binds to a loop in HD1-1 which connects the NB domain to the HD1 and WHD domains, providing orthogonal evidence for SS15-NRC interactions being mediated by this region. This loop was previously shown to act as a “hinge”, allowing the NB domain to move relative to the HD1 and WHD domains (FIG. 4). By binding to, and immobilizing, this hinge, SS15 likely abolishes conformational changes that are critical for NLR activation.

Moreover, as SS15 is able to suppress NRC1, NRC2 and NRC3, we leveraged the high degree of conservation that is characteristic of plant NB-ARC domains to narrow down the list of residues within the binding interface that could underpin this interaction. We shortlisted residues within the HD1-1 region that were similar in NRC1, NRC2 and NRC3 (FIG. 3B). Combining information from the co-crystal structure and the alignments allowed us to select 13 candidate residues to test by mutagenesis in NRC2 (FIG. 3B). We mutated each of these residues to the corresponding amino acid found in NRC4 and screened these NRC2 variants for susceptibility to SS15 inhibition in cell death assays. This revealed at least two variants, NRC2^{E316P} and NRC2^{D317K} which triggered cell death when activated by Rx/CP and were no longer inhibited by SS15 (FIG. 3C). We also tested all 13 single amino acid mutants for association with SS15 by in planta co-immunoprecipitation and found that NRC2^{D317K} and NRC2^{E316P} exhibited reduced association with SS15 relative to NRC2 (FIG. 3D), which is in line with the observation that SS15 is not able to suppress this variant (FIG. 3C). We conclude that the E316 and D317 residues are critical for SS15-mediated inhibition of NRC2 and that, in particular, mutating the D317 residue allows Rx/CP-activated NRC2 to evade SS15 association and inhibition.

We next tested whether these two SS15-evading variants retained correct functionality with other previously characterized NRC2-dependent sensor NLRs. We tested this by performing complementation assays in nrc2/3/4 CRISPR KO N. benthamiana plants with NRC2^{E316P} and NRC2^{D317K}, activating these helpers with a panel of agronomically important sensor NLRs mediating resistance to diverse pathogens. The panel included the potato cyst nematode R gene and Rx paralog Gpa2, as well as various oomycete and bacterial resistance proteins. In the absence of SS15, both NRC2 variants mediate cell death upon activation by all NRC2-dependent sensors tested (FIG. 5A). In the presence of SS15, the NRC2^{E316P} variant could evade SS15 suppression when activated by Rx but not when activated by all other sensors tested. The NRC2^{D317K} variant, in contrast, was able to evade SS15 inhibition regardless of the sensor NLR used to activate it. We therefore selected NRC2^{D317K} for follow-up biochemical studies, using the BN-PAGE-based resistosome formation assays discussed previously. Unlike Rx/CP-activated NRC2, Rx/CP-activated NRC2^{D317K} oligomerized in the presence of SS15 and exhibited no in vivo complex formation with the inhibitor (FIG. 5B). We conclude that NRC2^{D317K} is able to fully evade SS15-mediated immune suppression, retaining the capacity to oligomerize and mediate cell death when activated by multiple agronomically important sensor NLRs.

### Discussion

Our study reveals how a parasite effector has evolved as an inhibitor of a helper NLR by directly binding the HD1 region of its NB-ARC domain to prevent resistosome formation. By binding and immobilizing a critical hinge loop in the NB-ARC domain of these NLRs, SS15 restricts movement of the NB domain relative to the HD1 and WHD domains, preventing immune receptor activation. Remarkably, while SS15 can bind and inhibit NRC2. We made chimeric NRC2-NRC4 variants which, together with structural information, helped us identify the binding interface. Mutational studies of this interface allowed us to generate a single amino acid NRC2 (NRC2^{D317K}) variant that evades SS15 inhibition without compromising receptor signaling capacity. This NRC2^{D317K} variant can now support signaling by any NRC2-dependent sensor even in the presence of SS15.

By the approach described herein, cryptic or defeated resistance proteins can be resurrected to enhance disease resistance. Moreover, single amino acid NRC2 variants can be generated in-locus using gene editing technologies in agronomically important crop species, making deployment of this technology viable in countries where transgenic approaches are not feasible. In this work, we have developed a novel approach to achieve robust immunity by engineering NLRs that avoid parasite suppression. This can be applied to other plant NLR immune receptors that are directly targeted by parasite effectors. This technology holds the potential to unlock a new era in disease resistance breeding.

SEQUENCE LISTING

SEQ ID NO: 1; Nicotiana benthamiana NRC2a HD1 domain (NbNRC2a-HD1), amino
acid; GenBank accession number: ALQ52761.
PHDLKFLTEDESWILLEKKVFHKDKCPPELELSGKSIAKKCNGLPLAIVVIAGALIGKGK
TSREWKQVDESVGEHLINK
SEQ ID NO: 2; Nicotiana benthamiana NRC2a HD1-1 domain (NbNRC2a-HD1-1),
amino acid; GenBank accession number: ALQ52761.
PHDLKFLTEDESWILLEKKVFHKDKCPPELELSGKSIAK
SEQ ID NO: 3; Nicotiana benthamiana NRC2a RNBS-C motif, amino acid; Genbank
accession number: ALQ52761.
HDLKFLTEDESWILLEKKVF
SEQ ID NO: 4; Nicotiana benthamiana NRC2b HD1 domain (NbNRC2b-HD1), amino
acid; GenBank accession number: ALQ52762.
PHDLKFLTEDESWILLEKKVFHKDKCPPELELSGKSIAKKCNGLPLAIVVIAGALIGKGK
TSREWKQVDESVGEHLINK
SEQ ID NO: 5; Nicotiana benthamiana NRC2b HD1-1 domain (NbNRC2b-HD1-1),
amino acid; GenBank accession number: ALQ52762.
PHDLKFLTEDESWILLEKKVFHKDKCPPELELSGKSIAK
SEQ ID NO: 6; Nicotiana benthamiana NRC2b RNBS-C motif, amino acid; GenBank
accession number: ALQ52762.
HDLKFLTEDESWILLEKKVF
SEQ ID NO: 7; Nicotiana benthamiana NRC3 HD1 domain (NbNRC3-HD1), amino
acid; GenBank accession number: QER78240.
PHDLKFLTENESWELLEKRVFHKEKCPFELELPGKSIAKKCRGLPLAIVVIAGALIGKGK
TTREWELVADSVGEHLINR
SEQ ID NO: 8; Nicotiana benthamiana NRC3 HD1-1 domain (NbNRC3-HD1-1),
amino acid; GenBank accession number: QER78240.
PHDLKFLTENESWELLEKRVFHKEKCPFELELPGKSIAK
SEQ ID NO: 9; Nicotiana benthamiana NRC3 RNBS-C motif, amino acid; Genbank
accession number: QER78240.
HDLKFLTENESWELLEKRVF
SEQ ID NO: 10; Nicotiana benthamiana, NRC4 HD1 domain (NbNRC4-HD1), amino
acid; GenBank accession number: QER78241.
PHDLKFLTPKESFELLVKRVFGKKPCPKDLVGHGESIAGKCGGVPLAVVVIAGALRGR
PNTSDWIRVERNVVQHLYTNS
SEQ ID NO: 11; Nicotiana benthamiana, NRC4 HD1-1 domain (NbNRC4-HD1),
amino acid; GenBank accession number: QER78241.
PHDLKFLTPKESFELLVKRVFGKKPCPKDLVGHGESIAG
SEQ ID NO: 12; Nicotiana benthamiana, NRC4 RNBS-C motif, amino acid; GenBank
accession number: QER78241.
HDLKFLTPKESFELLVKRVF
SEQ ID NO: 13: Solanum tuberosum NRC2 HD1 domain (StNRC2-HD1), amino acid;
NCBI Reference Sequence: XP_006359790.1.
PHDLKFLSEDESWILLEKKVFHKDKCPPELVVPSGKSIAKKCKGLPLAIVVIAGALIGKG
KTPREWKQVDDSVSEHLINR
SEQ ID NO: 14; Solanum tuberosum NRC2 HD1-1 domain (StNRC2-HD1-1), amino
acid; NCBI Reference Sequence: XP_006359790.1.
PHDLKFLSEDESWILLEKKVFHKDKCPPELVVPSGKSIAK
SEQ ID NO: 15; Solanum tuberosum NRC2 RNBS-C motif, amino acid; NCBI
Reference Sequence: XP_006359790.
HDLKFLSEDESWILLEKKVF
SEQ ID NO:16; Solanum tuberosum NRC3 HD1 domain (StNRC3-HD1), amino acid;
NCBI Reference Sequence: XM_006362512.2
PHDLKFLTEDESWELLEKKVFHKEKCPPELELPGKSIAEKCMGLPLAIVVIAGALIGKGK
TTREWELVAASVREHLINR
SEQ ID NO: 17; Solanum tuberosum NRC3 HD1-1 domain (StNRC3-HD1-1), amino
acid; NCBI Reference Sequence: XM_006362512.2
PHDLKFLTEDESWELLEKKVFHKEKCPPELELPGKSIAE
SEQ ID NO: 18; Solanum tuberosum NRC3 RNBS-C motif, amino acid; NCBI
Reference Sequence: XM_006362512.2
DLKFLTEDESWELLEKKVF
SEQ ID NO: 19; Solanum tuberosum NRC4 HD1 domain (StNRC4-HD1), amino acid;
NCBI Reference Sequence: XP_006367034.1
PHVLKFLKTEESFELLVMRVFGKGSCPNELVVTGKKIAEKCGGVPLVVVVIAGALRGR
SDKKDWERVDKNVVQHFGEHT
SEQ ID NO: 20; Solanum tuberosum NRC4 HD1-1 domain (StNRC4-HD1-1), amino
acid; NCBI Reference Sequence: XP_006367034.1
PHVLKFLKTEESFELLVMRVFGKGSCPNELVVTGKKIAE
SEQ ID NO: 21; Solanum tuberosum NRC4 RNBS-C motif, amino acid; NCBI
Reference Sequence: XP_006367034.1
HELKFLEKEESFELLVMRVF
SEQ ID NO: 22; Solanum lycopersicum, NRC1 HD1 domain (SINRC1-HD1), amino
acid; NCBI Reference Sequence: NP_001234202.
PHDLKFLTDEESWILLEKRAFHKAKCLPELETNGKSIARKCKGLPLAIVVIAGALIGKSK
TIKEWEQVDQSVGEHFINR
SEQ ID NO: 23; Solanum lycopersicum, NRC1 HD1-1 domain (SINRC1-HD1-1),
amino acid; NCBI Reference Sequence: NP_001234202.
PHDLKFLTDEESWILLEKRAFHKAKCLPELETNGKSIAR
SEQ ID NO: 24; Solanum lycopersicum, NRC1 RNBS-C motif, amino acid; NCBI
Reference Sequence: NP_001234202.
HDLKFLTDEESWILLEKRA
SEQ ID NO: 25; Solanum lycopersicum, NRC2 HD1 domain (SINRC2-HD1), amino
acid; NCBI Reference Sequence: XP_004248798.
PHDLKFLTEDESWILLEKKVFHKDKCPPELVLSGKSIAKKCKGLPLAIVVIAGALIGKGK
TPREWKQVDDSVSEHLINR
SEQ ID NO: 26; Solanum lycopersicum, NRC2 HD1-1 domain (SINRC2-HD1-1),
amino acid; NCBI Reference Sequence: XP_004248798.
PHDLKFLTEDESWILLEKKVFHKDKCPPELVLSGKSIAK
SEQ ID NO: 27; Solanum lycopersicum, NRC2 RNBS-C motif, amino acid; NCBI
Reference Sequence: XP_004248798.
HDLKFLTEDESWILLEKKVF
SEQ ID NO: 28; Solanum lycopersicum, NRC3 HD1 domain (SINRC3-HD1), amino
acid; NCBI Reference Sequence: XP_004238948.
PHDLKFLTKDESWELLEKKVFHKEKCPPELELPGISIAEKCMGLPLAIVVIAGALIGKGK
TTREWELVAASVGEHLINR
SEQ ID NO: 29; Solanum lycopersicum, NRC3 HD1-1 domain (SINRC3-HD1-1),
amino acid; NCBI Reference Sequence: XP_004238948.
PHDLKFLTKDESWELLEKKVFHKEKCPPELELPGISIAE
SEQ ID NO: 30; Solanum lycopersicum, NRC3 RNBS-C motif, amino acid; NCBI
Reference Sequence: XP_004238948.
HDLKFLTKDESWELLEKKVF
In SEQ ID NOs: 32, 34, 36, 41, 43, 47, 49, and 51, bold bases represent NB-ARC
(HD1) domain, underlined represents NB-ARC1 (HD1-1) subdomain, and italics
represents RNBS-C motif. In SEQ ID Nos: 31, 33, 35, 40, 42, 46, 48, and 50,
underlined represents NB-ARC1 (HD1-1) subdomain. In SEQ ID Nos: 37, 38, 39,
44, 45, and 52-59, mutated residues are underlined and highlighted in separate
sequences.
SEQ ID NO: 31; Nicotiana benthamiana, NbNRC2a cDNA; GenBank accession
number: KT936525.
ATGGCGAACGTTGCGGTGGAGTTTCTGGTGCAGAACTTGATGCAGCTGCTAAGAG
ACAATGCGGAGCTGATTGTTGGAGTTAAGGATTCGGCTGAGAGTCTGCTTCAAGA
TCTCAATGATTTCAACGCTTTTCTCAAGCAAACTGCTAAGTCCCGCACTGAGAACG
ATGTCCACAAAGAATTGGTGAAGAAAATTAAGACTGTGGTCAACTCTGCTGAAGAT
GCCATTGATAAGTTTGTGATTGAGGCTAAGCTTCACAAGGACAAAGGGGTTGGCA
GATTTGTGGATGTTAAGCATTATAAAAGAGTGTATGATGTAGCAGGGGAGATTAAA
ACTATTAGAGACAAAGTCAAAGAAATTCGTCTCAATAATGCACTTGACCTTCAGGC
CCTTCAAGATGAAGATCAATCTGCCAAAGGTGTCCAAGAAAGAAAGCCTCCGGTG
GTAGAGGAAGATGATGTGGTTGGATTTGAAGAGGAAGCAGATAAAGTAATCAACC
GTCTTCTGGGAGGATCGAGTGGACTAGAAGTTGTTCCAGTTGTTGGAATGCCTGG
TCTCGGCAAAACGACACTGGCAAATAAGATTTACAAGCATCCTGACATCGGGTAC
CAGTTTTTTACTCGCATTTGGGTTTATGTTTCTCAATCATACAGGAGAAGAGAATTA
TTTCTCAACATCATCAGCAAATTCACTCGCAATACCAAACAATACCACGATATGTGT
GAGGAGGATTTAGCTGATGAAATAGAAGATTTTTTGGGCAAGGGAGGGAAATACT
TGATTGTCTTGGATGATGTATGGTCTCCTGACGCTTGGGAACGTATCAGAATAGCT
TTCCCAAACAACAACAAATCCAATAGAATATTATTGACCACCCGAGATAGCAAAGT
TGCTAAGCAATGCAAGCAGTGCATTGGTATACCTCATGATTTAAAATTTCTGACTG
AAGATGAAAGTTGGATTTTACTGGAGAAGAAAGTTTTCCATAAAGATAAATGTCCT
CCTGAATTGGAACTATCTGGAAAGAGCATAGCCAAAAAATGTAATGGACTACCCCT
TGCAATTGTTGTTATTGCAGGAGCACTAATTGGGAAAGGTAAGACATCAAGAGAGT
GGAAACAAGTGGATGAGAGTGTGGGTGAACACCTCATAAATAAAGACCAGCCTGA
GAATTGTAACAAATTGGTGCAACTGAGTTATGATCGCTTGTCTTACGACTTGAAAG
CATGTTTTTTGTATTGTGGTGCATTTCCTGGAGGCTTTGAGATCCCTGCTTGGAAG
CTAATCCGTTTGTGGATCGCAGAAGGGTTCATACAGTATAAAGGCCACTTATCTCT
TGAGTGTAAAGCAGAGGATAACTTGAATGATCTAATTAACAGGAATCTAGTGATGG
TAATGCAAAGAACATCTGATGGTCAAATCAAAACATGTCGTCTTCATGACATGTTG
CACGAGTTTTGCAGACAAGAGGCTATGAAGGAAGAAAATCTTTTCCAAGAAATAAA
ACTAGGTGCTGAGCAATATTTCCCTGGAAAACGGGAACTAGCCACCTACCGTCGC
TTATGCATTCATTCCTCAGTTTTGGAATTTATCTCTACAAAGCCCTCAGGTGAACAT
GTCAGGTCATTCTTATCTTTTTCTTTAAAAAAGATTGAGATGCCATCTGTCGACATC
CCAACCATACCAAAAGGCTTTCCGTTGCTGAGGGTTTTTGATGTCGAGTCCATCAA
CTTCAGTCGCTTTTCCAAGGAGTTTTTCCAGTTATATCATTTGAGGTATATTGCTTT
CTCATCTGACACAATCAAGATCATTCCTAAACACATTGGAGAACTGTGGAACATCC
AAACACTCATAATTAACACGCAACAACGCTCTCTTGATATCCAAGCAAACATATGG
AATATGGAACGACTAAGGCATCTGCACACTAACTCTTCTGCTAAATTGCCTGTTCC
TGTGACCCCAAGAAGTAGTAAAGTTCCTTTGGTAAATCAAAGCCTGCAAACTCTCT
CCACCATTGCTCCCGAAAGCTGCACAGAAGAAGTGTTTGCAAGGACTCCAAACCT
GAAAAAACTGGGTATTCGTGGGAAAATAGCTGTGCTTCTTGAACCTAATAAGTCAT
TGTTAAAAAATGTGAAGAAACTAGAATCCCTTGAAAACTTGAAGCTGATAAATGATA
GTAGTCAAACAGGAAAAGGGTTACGCCTTCCTCCCTCATATATATTTCCCACGAAG
TTGAGGAAGCTATCTTTAGTAGATACCTGGCTGGAGTGGAATGATATGTCTATATT
GGGACAGATGGAACACCTTGAAGTCCTGAAGCTGAAAGAAAATGGGTTTATGGGA
GAATGCTGGGAGTCAGTTGGAGGTTTTTGTTCCCTACTGGTGCTGTGGATTGAAA
GGACAGATTTAGTTTCTTGGAAAGCATCAGCTGATCACTTTCCAAGACTTAAGCAT
CTTGTTCTCATCTGCTGCGATAAGCTTAAGGAAATCCCCATTGGCCTGGCTGATAT
ACGCAGCTTCCAAGTGATGGAGTTGCAAAACTCAACCAAAACAGCAGCAATATCT
GCACGGGGAATACGAGACAAAAAAGACAAGCAAACTCAAGAAGGGACTAACAATA
ATGGGTTCAAGCTCTCTATATTCCCTCCCGATCTCTAA
SEQ ID NO: 32; Nicotiana benthamiana, NbNRC2a amino acid; GenBank accession
number: ALQ52761.1.
MANVAVEFLVQNLMQLLRDNAELIVGVKDSAESLLQDLNDFNAFLKQTAKSRTENDV
HKELVKKIKTVVNSAEDAIDKFVIEAKLHKDKGVGRFVDVKHYKRVYDVAGEIKTIRDK
VKEIRLNNALDLQALQDEDQSAKGVQERKPPVVEEDDVVGFEEEADKVINRLLGGSS
GLEVVPVVGMPGLGKTTLANKIYKHPDIGYQFFTRIWVYVSQSYRRRELFLNIISKFTR
NTKQYHDMCEEDLADEIEDFLGKGGKYLIVLDDVWSPDAWERIRIAFPNNNKSNRILLT
TRDSKVAKQCKQCIGIPHDLKFLTEDESWILLEKKVFHKDKCPPELELSGKSIAKKCN
GLPLAIVVIAGALIGKGKTSREWKQVDESVGEHLINKDQPENCNKLVQLSYDRLSYDL
KACFLYCGAFPGGFEIPAWKLIRLWIAEGFIQYKGHLSLECKAEDNLNDLINRNLVMVM
QRTSDGQIKTCRLHDMLHEFCRQEAMKEENLFQEIKLGAEQYFPGKRELATYRRLCIH
SSVLEFISTKPSGEHVRSFLSFSLKKIEMPSVDIPTIPKGFPLLRVFDVESINFSRFSKEF
FQLYHLRYIAFSSDTIKIIPKHIGELWNIQTLIINTQQRSLDIQANIWNMERLRHLHTNSSA
KLPVPVTPRSSKVPLVNQSLQTLSTIAPESCTEEVFARTPNLKKLGIRGKIAVLLEPNKS
LLKNVKKLESLENLKLINDSSQTGKGLRLPPSYIFPTKLRKLSLVDTWLEWNDMSILGQ
MEHLEVLKLKENGFMGECWESVGGFCSLLVLWIERTDLVSWKASADHFPRLKHLVLI
CCDKLKEIPIGLADIRSFQVMELQNSTKTAAISARGIRDKKDKQTQEGTNNNGFKLSIFP
PDL
SEQ ID NO: 33; Nicotiana benthamiana, NbNRC2b cDNA; GenBank accession
number: KT936526.
ATGGCGAACGTTGCGGTGGAATTTTTGGTGCAGAACTTGATGCAGCTGCTGAGGG
ACAATGCGGAGCTGATTATTGGGGTTAAGGATTCGGCTGAGAGCCTGCTTCAAGA
TCTCAATGATTTCAACGCTTTTCTCAAGCAAGCTGCCAAGTCCCGCATTGAGAACG
ATGTCCACAAAGAATTGGTGAAGAAAATTAAGACAATGGTCAACTCTGCTGAAGAT
GCCATTGATAAGTTTGTAATTGAGGCTAAGCTTCACAAGGACAAAGGGGTTGGCA
GATTTGTAGATGTTAAGCATTATAAAAGAGTGTATGATGTAGCAGCCGAGATCAAA
GGTATCAGAGAAAAAGTGAAAGAAATCCGTCAGAATAATGCCCTTGACCTTCAAG
CCCTTCAAGATGAAGATCAATCTGCCAAAGGTGTCGAAGAAAGAAAGCCTCCAGT
GGTAGAGGAAGATGATGTGGTTGGATTTGAAGAGGAAGCAGATAAAGTAATCAAC
CGTCTTCTGGGAGGATTGAGTGGACTAGAAGTTGTTCCAGTTGTTGGAATGCCTG
GTCTCGGCAAAACGACGCTAGCAAATAAGATTTACAAGCATCCTGACATCGGGTA
CCAGTTTTTTACTCGCATTTGGGTTTATGTTTCTCAATCATACAGGAGAAGAGAATT
ATTTCTCAACATCATCAGCAAATTCACTCGAAATACCAAACAATACCATGATATGTG
TGAGGAGGATTTAGCTGATGAAATACAAGATTTTTTGGGCAAAGGAGGAAAATACT
TGATTGTCTTGGATGATGTATGGTCTCCTGACGCTTGGGAACACATCAAAATAGCT
TTCCCGAACAACAACAAATCCAATAGAATATTATTGACCACTCGAGATAGCAAAGT
TGCTAAGCAATGCAAGCAGTGCATTGGTATACCTCATGATTTAAAATTTCTGACTG
AAGATGAAAGTTGGATTTTACTGGAGAAGAAAGTTTTCCATAAAGATAAATGTCCT
CCTGAATTGGAACTATCTGGAAAGAGCATAGCCAAAAAATGTAATGGACTACCCCT
TGCGATTGTTGTTATTGCAGGAGCACTAATTGGGAAAGGTAAGACATCAAGAGAG
TGGAAACAAGTGGATGAGAGTGTGGGTGAACACCTCATAAATAAAGACCAGCCTG
AGAATTGTAACAAATTGGTGCAACTGAGTTATGATCGCTTGTCTTATGACTTGAAA
GCATGTTTTTTATATTGTGGTGCATTTCCCGGAGGCTTTGAGATCCCTGCTTGGAA
GTTAATCCGTTTGTGGATCGCAGAAGGGTTCATACAGTATAAAGGCCACTTATCTC
TTGAGTGTAAAGCAGAGGATAACTTGAATGATCTCATCAACAGGAATCTAGTGATG
GTAATGCAAAGGACATCTGATGGGCAAATCAAAACATGTCGTCTTCATGACATGTT
GCACGAGTTTTGCAGACAAGAGGCGATGAAGGAAGAAAATCTTTTCCAAGAAATA
AAATTAGGTGCTGAGCAATATTTCCCAGGAAAACGGGAACTAGCCACCTACCGTC
GCTTATGCATTCATTCCTCAGTTTTGGAATTTATCTCTACAAAGCCCTCAGGTGAA
CATGTCAGGTCATTCTTATCTTTTTCTTTAAAAAAGGTCGAGATGCCATCTGTCGAC
ATCCCAACCATACCAAAAGGCTTCCCGTTGCTGAGGGTTTTTGATGTCGAGTCCAT
CAACTTCAGTCGCTTTTCCAAGGAGTTTTTCCAGTTATATCATTTGAGGTATATTGC
TTTCTCATCTGACACAATCAAGATCATTCCTAAACACATTGGAGAACTGTGGAACA
TCCAAACACTCATAATTAACACGCAACAACACTCTCTTGATATCCAAGCAAACATAT
GGAATATGGCACGACTAAGGCATCTGCACACTAACTCTTCTGCTAAATTGCCTGTT
CCTGTGACCCCAAGAAGTAGTAAAGTTCCTTTGGTAAATCAAAGCCTGCAAACTCT
CTCCACCATTGCTCCCGAAAGCTGCACAGAAGAAGTGTTTGCAAGGACTCCAAAC
CTGAAAAAGTTGGGTATTCGTGGGAAAGTTGCTGTGCTTCTTGAACCTAATAAGTC
ATTGTTAAAAAATGTGAAGAAACTAGAATCCCTTGAAAACTTGAAGCTGATAAATGA
TAGTAGTCAAACAGGAAAAGGGTTACGCCTTCCACCCTCATATATATTTCCCACGA
AGTTGAGGAAGCTATCTTTAGTAGATACCTGGCTGGAGTGGAACGATATGTCTATA
TTGGGGCAGATGGAACACCTTGAAGTTCTGAAGCTGAAAGAAAATGGGTTTATGG
GAGAATGCTGGGAGTCAGTTGGAGGTTTTTGTTCCCTATTGGTGTTGTGGATTGA
AAGGACAGACTTAGTTTCTTGGAAAGCATCAGCTGATCACTTTCCAAGACTTAAGC
ATCTTGTTCTCATCTGCTGCGATAAGATTAAGGAAATCCCCATTGGCCTGGCTGAT
ATACACAGCTTCCAAGTGATGGAGTTGCAAAACTCCACCAAAACAGCAGCAATATC
TGCACGGGAAATACGAGACAAAAAAGACAAGCAAACTCAAGAGGGGACTAACAAC
AATGGGTTCAAGCTCTCTATATTCCCTCCCGATCTCTGA
SEQ ID NO: 34; Nicotiana benthamiana, NbNRC2b cDNA; GenBank accession
number: ALQ52762.
MANVAVEFLVQNLMQLLRDNAELIIGVKDSAESLLQDLNDFNAFLKQAAKSRIENDVH
KELVKKIKTMVNSAEDAIDKFVIEAKLHKDKGVGRFVDVKHYKRVYDVAAEIKGIREKV
KEIRQNNALDLQALQDEDQSAKGVEERKPPVVEEDDVVGFEEEADKVINRLLGGLSG
LEVVPVVGMPGLGKTTLANKIYKHPDIGYQFFTRIWVYVSQSYRRRELFLNIISKFTRN
TKQYHDMCEEDLADEIQDFLGKGGKYLIVLDDVWSPDAWEHIKIAFPNNNKSNRILLTT
RDSKVAKQCKQCIGIPHDLKFLTEDESWILLEKKVFHKDKCPPELELSGKSIAKKCNG
LPLAIVVIAGALIGKGKTSREWKQVDESVGEHLINKDQPENCNKLVQLSYDRLSYDLK
ACFLYCGAFPGGFEIPAWKLIRLWIAEGFIQYKGHLSLECKAEDNLNDLINRNLVMVMQ
RTSDGQIKTCRLHDMLHEFCRQEAMKEENLFQEIKLGAEQYFPGKRELATYRRLCIHS
SVLEFISTKPSGEHVRSFLSFSLKKVEMPSVDIPTIPKGFPLLRVFDVESINFSRFSKEF
FQLYHLRYIAFSSDTIKIIPKHIGELWNIQTLIINTQQHSLDIQANIWNMARLRHLHTNSSA
KLPVPVTPRSSKVPLVNQSLQTLSTIAPESCTEEVFARTPNLKKLGIRGKVAVLLEPNK
SLLKNVKKLESLENLKLINDSSQTGKGLRLPPSYIFPTKLRKLSLVDTWLEWNDMSILG
QMEHLEVLKLKENGFMGECWESVGGFCSLLVLWIERTDLVSWKASADHFPRLKHLVL
ICCDKIKEIPIGLADIHSFQVMELQNSTKTAAISAREIRDKKDKQTQEGTNNNGFKLSIFP
PDL
SEQ ID NO: 35; Nicotiana benthamiana, NbNRC3 cDNA; GenBank accession
number: MK692736.
ATGGCAGATGTAGCAGCAGATGTAGCAGTAAAATTCCTAGTAGAAAACTTGATGCA
ATTATTAATCGACAACGCTGACTTGATTATTGGTATAAAGGGTGAAGTTGAAAATTT
ACTACAAGATCTGAATGATTTCAATGCTTTTCTCAAACAAGCAGCTAAATCAAGGA
GAGACAATGAAGTCTTGAAATCACTAGTAAAGAAGATAAGGAAAGTGGTAAATGAT
GCTGAAGATTCAATTGATAAGTTTGTGATTGAAGCTAAAAGACATGACGATAAAAA
TAAATTTGCTCAGTGGTTTCATCTTACTCATGTTGCTAGAGCAAAAGGGGTTGCTG
ATGAGATTAAAACTATAAGGGAAAGGGTGAAGGAAATTCGCCAAAATGATGCTTAT
GGACTTCAAGCTATAACTTCTTATGATAATTTCAACCAAGGTGCTCAGGAGAGGAA
GGTCCCTGTAGTTGAGGAAGACGACGTGGTAGGCTTTGATGACGAAGCGAAAACT
GTAATTGATCGCCTCATTGGAGGATCAGATTATGTTGTGCCGGTCGTTGGTATGC
CTGGTCTTGGAAAAACAACTTTGGCATATAAGATTTTCAAGGATTCCACAGTTGAG
TATGAGTTTTTCAACCGCATATGGGTATATGTCTCTCAATCATTCAACAGAAGGGA
AATATTTCTCAACATCATCAGCAAATTCACTCGAAACACCAAACAATACCATGATAC
ACCAGAGGAGGAATTAGCAAATGAAATAAAGGAGCTGCTTGGGAAGGGTGGGAA
ATATCTTGTTGTTTTAGATGATGTGTGGACAAGAGAAGCTTGGGATCGCATTAAAA
TTGCTTTCCCCAATAATAATAAACGGAATAGAGTCTTGATGACTACTAGACAAAAC
AATGTGGCTAAGTCTTGCAACGATAAACCTCATGATCTAAAGTTTTTGACTGAAAAT
GAAAGTTGGGAGCTACTTGAGAAGAGAGTTTTTCACAAGGAAAAATGTCCATTTGA
GTTAGAATTACCTGGAAAAAGTATAGCCAAAAAATGTAGGGGCTTACCACTTGCAA
TAGTTGTCATTGCTGGAGCTTTAATAGGCAAAGGAAAGACGACAAGGGAGTGGGA
GCTGGTGGCTGACAGTGTGGGTGAGCACCTCATAAATAGAGATCCAGAGAACTG
CAAGAAATTGGTGCAGATGAGTTATGACCGTTTGCCTTATGATTTAAAGGCGTGCT
TCTTATATTGTGGGGCTTTTCCTGGCGGTTCTGAAATTTCAGCTCAGAAGTTGATT
TGTTTATGGATTGCGGAAGGATTCATACAATACCAAGGACCATTGACTCTAGAGGA
TATAGCAGAGGACCACTTGAATGATCTTGTCAATAGGAACTTAGTGATGGTAATGA
AAAGGAGTTCTAGCGGTCAAATCAAAACATGTCGTGTTCATGACATGTTGCATGAG
TTCTGCAGACATGAAGCTATGATGGAGGAAAATCTATTCCAAGAAATCAAACGAGG
GCAAGAACATTCTTTTCCAGAGAAACAAGAGTTAGCCTCTTATCGTCGCTTGTGCA
TTCATTCGTCTGTTTCTGAATTCTTGTCAACAAAACCCTTTGCTGAACATGTTAGGT
CGTTCTTATGCTTTGCCTCGAAAAAATTTGAGATGCCTTTAGGTGAAATACCAGCC
ATCCCCAGAGCCTTTCCCTTGCTTAGGGTACTCGATGCTGAATCAATCAAGTTCAG
TCGTTTTTCTAGAGAGTTCTTCAAATTATTCCATTTGAGGTACATTGCTTTCTCAAC
TGACTCGATTATGACCATTCCTACAAACATTGGGAATCTTTGGAACGTACAAACAC
TTATAATTGAGACACAACAGGGTACTCTTGACATTAAAGCAGACATTTGGAATATG
ACAAGATTAAGGCATGTGTGCATAAACGCCTCTGCTACATTGCCTTCCCCTAAGC
GCCCCAAGAGTAGCAAGGACAACTTGGTGAATCGTTGCCTACAAACACTTTCTAC
CATAGCACCTGAATGTTGCACGGCAGAAGTTTTTACTAGAACTCCTAATCTCAAGA
AATTAGGTGTTCGTGGAAAAATAGATGCACTTCTAGAAACCAGTAAGGACGGGTC
CAGTTCTGGTTTGTTCAGCAACATAGGAAAGTTAGATTGCCTCGAAAAGCTGAAGT
TGGTAAATGATACTCGTCAAAGTAGAAAACAGCTACACCTTCCTCCAGCATATATT
TTTCCTCAGAAGCTAAAAAAACTCACTCTGATAGATACGTGGTTCGAATGGAAAGA
TATGTCTATATTAGGTCTATTAGAGTACCTTGAAGTGCTGAAGTTGAAGGAAAATG
CGTTTAGGGGACAGTCGTGGGAACCAGAGGATAGTGGTTTTCCTCGTCTGCAGGT
CCTATGGATTGAAAGGACAGATCTATCTTCTTGGAAGGCCTCATCTGGAAACTTTC
CACGACTAAAATGCCTTGTTCTTATAGCATGTGATAATCTTAAGGAACTACCAGCT
GAACTGGCTGATGTGGAAAACCTCCAGTTAATGGAACTCCAGAGTACCAGTGTGT
CTGCTGCTAAATCTGCACGAGCAATACTAAAGAAGAAACAACAAAAAGTTGGCAG
CGGATTCAAGCTTTCTGTATTTCCTCCAGATCTCGGATTGTAA
SEQ ID NO: 36; Nicotiana benthamiana, NbNRC3 amino acid; GenBank accession
number: QER78240.
MADVAADVAVKFLVENLMQLLIDNADLIIGIKGEVENLLQDLNDFNAFLKQAAKSRRDN
EVLKSLVKKIRKVVNDAEDSIDKFVIEAKRHDDKNKFAQWFHLTHVARAKGVADEIKTI
RERVKEIRQNDAYGLQAITSYDNFNQGAQERKVPVVEEDDVVGFDDEAKTVIDRLIGG
SDYVVPVVGMPGLGKTTLAYKIFKDSTVEYEFFNRIWVYVSQSFNRREIFLNIISKFTRN
TKQYHDTPEEELANEIKELLGKGGKYLVVLDDVWTREAWDRIKIAFPNNNKRNRVLMT
TRQNNVAKSCNDKPHDLKFLTENESWELLEKRVFHKEKCPFELELPGKSIAKKCRG
LPLAIVVIAGALIGKGKTTREWELVADSVGEHLINRDPENCKKLVQMSYDRLPYDLKA
CFLYCGAFPGGSEISAQKLICLWIAEGFIQYQGPLTLEDIAEDHLNDLVNRNLVMVMKR
SSSGQIKTCRVHDMLHEFCRHEAMMEENLFQEIKRGQEHSFPEKQELASYRRLCIHS
SVSEFLSTKPFAEHVRSFLCFASKKFEMPLGEIPAIPRAFPLLRVLDAESIKFSRFSREF
FKLFHLRYIAFSTDSIMTIPTNIGNLWNVQTLIIETQQGTLDIKADIWNMTRLRHVCINAS
ATLPSPKRPKSSKDNLVNRCLQTLSTIAPECCTAEVFTRTPNLKKLGVRGKIDALLETS
KDGSSSGLFSNIGKLDCLEKLKLVNDTRQSRKQLHLPPAYIFPQKLKKLTLIDTWFEWK
DMSILGLLEYLEVLKLKENAFRGQSWEPEDSGFPRLQVLWIERTDLSSWKASSGNFP
RLKCLVLIACDNLKELPAELADVENLQLMELQSTSVSAAKSARAILKKKQQKVGSGFKL
SVFPPDLGL
SEQ ID NO: 37; Nicotiana benthamiana, NRC2a^{D317K} amino acid.
MANVAVEFLVQNLMQLLRDNAELIVGVKDSAESLLQDLNDFNAFLKQTAKSRTENDV
HKELVKKIKTVVNSAEDAIDKFVIEAKLHKDKGVGRFVDVKHYKRVYDVAGEIKTIRDK
VKEIRLNNALDLQALQDEDQSAKGVQERKPPVVEEDDVVGFEEEADKVINRLLGGSS
GLEVVPVVGMPGLGKTTLANKIYKHPDIGYQFFTRIWVYVSQSYRRRELFLNIISKFTR
NTKQYHDMCEEDLADEIEDFLGKGGKYLIVLDDVWSPDAWERIRIAFPNNNKSNRILLT
TRDSKVAKQCKQCIGIPHDLKFLTEKESWILLEKKVFHKDKCPPELELSGKSIAKKCNG
LPLAIVVIAGALIGKGKTSREWKQVDESVGEHLINKDQPENCNKLVQLSYDRLSYDLKA
CFLYCGAFPGGFEIPAWKLIRLWIAEGFIQYKGHLSLECKAEDNLNDLINRNLVMVMQ
RTSDGQIKTCRLHDMLHEFCRQEAMKEENLFQEIKLGAEQYFPGKRELATYRRLCIHS
SVLEFISTKPSGEHVRSFLSFSLKKIEMPSVDIPTIPKGFPLLRVFDVESINFSRFSKEFF
QLYHLRYIAFSSDTIKIIPKHIGELWNIQTLIINTQQRSLDIQANIWNMERLRHLHTNSSAK
LPVPVTPRSSKVPLVNQSLQTLSTIAPESCTEEVFARTPNLKKLGIRGKIAVLLEPNKSL
LKNVKKLESLENLKLINDSSQTGKGLRLPPSYIFPTKLRKLSLVDTWLEWNDMSILGQM
EHLEVLKLKENGFMGECWESVGGFCSLLVLWIERTDLVSWKASADHFPRLKHLVLIC
CDKLKEIPIGLADIRSFQVMELQNSTKTAAISARGIRDKKDKQTQEGTNNNGFKLSIFPP
DL
SEQ ID NO: 38; Nicotiana benthamiana, NRC2b^{D317K} amino acid.
MANVAVEFLVQNLMQLLRDNAELIIGVKDSAESLLQDLNDFNAFLKQAAKSRIENDVH
KELVKKIKTMVNSAEDAIDKFVIEAKLHKDKGVGRFVDVKHYKRVYDVAAEIKGIREKV
KEIRQNNALDLQALQDEDQSAKGVEERKPPVVEEDDVVGFEEEADKVINRLLGGLSG
LEVVPVVGMPGLGKTTLANKIYKHPDIGYQFFTRIWVYVSQSYRRRELFLNIISKFTRN
TKQYHDMCEEDLADEIQDFLGKGGKYLIVLDDVWSPDAWEHIKIAFPNNNKSNRILLTT
RDSKVAKQCKQCIGIPHDLKFLTEKESWILLEKKVFHKDKCPPELELSGKSIAKKCNGL
PLAIVVIAGALIGKGKTSREWKQVDESVGEHLINKDQPENCNKLVQLSYDRLSYDLKA
CFLYCGAFPGGFEIPAWKLIRLWIAEGFIQYKGHLSLECKAEDNLNDLINRNLVMVMQ
RTSDGQIKTCRLHDMLHEFCRQEAMKEENLFQEIKLGAEQYFPGKRELATYRRLCIHS
SVLEFISTKPSGEHVRSFLSFSLKKVEMPSVDIPTIPKGFPLLRVFDVESINFSRFSKEF
FQLYHLRYIAFSSDTIKIIPKHIGELWNIQTLIINTQQHSLDIQANIWNMARLRHLHTNSSA
KLPVPVTPRSSKVPLVNQSLQTLSTIAPESCTEEVFARTPNLKKLGIRGKVAVLLEPNK
SLLKNVKKLESLENLKLINDSSQTGKGLRLPPSYIFPTKLRKLSLVDTWLEWNDMSILG
QMEHLEVLKLKENGFMGECWESVGGFCSLLVLWIERTDLVSWKASADHFPRLKHLVL
ICCDKIKEIPIGLADIHSFQVMELQNSTKTAAISAREIRDKKDKQTQEGTNNNGFKLSIFP
PDL
SEQ ID NO: 39; Nicotiana benthamiana, NRC3^{N317K} amino acid.
MADVAADVAVKFLVENLMQLLIDNADLIIGIKGEVENLLQDLNDFNAFLKQAAKSRRDN
EVLKSLVKKIRKVVNDAEDSIDKFVIEAKRHDDKNKFAQWFHLTHVARAKGVADEIKTI
RERVKEIRQNDAYGLQAITSYDNFNQGAQERKVPVVEEDDVVGFDDEAKTVIDRLIGG
SDYVVPVVGMPGLGKTTLAYKIFKDSTVEYEFFNRIWVYVSQSFNRREIFLNIISKFTRN
TKQYHDTPEEELANEIKELLGKGGKYLVVLDDVWTREAWDRIKIAFPNNNKRNRVLMT
TRQNNVAKSCNDKPHDLKFLTEKESWELLEKRVFHKEKCPFELELPGKSIAKKCRGLP
LAIVVIAGALIGKGKTTREWELVADSVGEHLINRDPENCKKLVQMSYDRLPYDLKACFL
YCGAFPGGSEISAQKLICLWIAEGFIQYQGPLTLEDIAEDHLNDLVNRNLVMVMKRSSS
GQIKTCRVHDMLHEFCRHEAMMEENLFQEIKRGQEHSFPEKQELASYRRLCIHSSVS
EFLSTKPFAEHVRSFLCFASKKFEMPLGEIPAIPRAFPLLRVLDAESIKFSRFSREFFKL
FHLRYIAFSTDSIMTIPTNIGNLWNVQTLIIETQQGTLDIKADIWNMTRLRHVCINASATL
PSPKRPKSSKDNLVNRCLQTLSTIAPECCTAEVFTRTPNLKKLGVRGKIDALLETSKDG
SSSGLFSNIGKLDCLEKLKLVNDTRQSRKQLHLPPAYIFPQKLKKLTLIDTWFEWKDMS
ILGLLEYLEVLKLKENAFRGQSWEPEDSGFPRLQVLWIERTDLSSWKASSGNFPRLKC
LVLIACDNLKELPAELADVENLQLMELQSTSVSAAKSARAILKKKQQKVGSGFKLSVFP
PDLGL
SEQ ID NO: 40; Solanum tuberosum StNRC2, cDNA. NCBI Reference Sequence:
XM_006359728.2
ATGGCGAACGTAGCAGTAGAATTTCTCGTTGAGAACTTGATGCAGCTGCTGCGGG
ACAACGCAGAGCTAATTAGTGGAGTTAAAGAGGCTGCTGAGAGTCTACTTCAAGA
TCTAAATGATTTCAACGCCTTTCTTAAGCAAGCTGCCAAGTGCCACATCAACGAGA
ACGAAGTTCTGAGAGAACTCGTCAAGAAAATCAGAACAGTGGTTAACTCTGCTGA
AGATGCTATTGATAAATTTGTGATTGAAGCTAAGCTACACAAGGATAAGGGTATGA
CCAGAGTATTAGACCTTCCCCATTATAAAAGAGTCAGGGAGGTAGCTGGTGAGAT
CAAAGCTATACGAAACAAAGTCAAAGAGATCAGACAGAATGATGCCATTGGACTTC
AGGCCCTTCAAGATGATGATTCATCTGCCAGAGGTTTCGAAGAAAGAAAGCCTCC
AGTGGTAGAGGAAGATGATGTGGTTGGATTTGACGAAGAAGCAGATATTGTAATC
AAACGCCTTCTTGGAGAATCAAATCGTCTAGAAGTTGTTCCAGTTGTTGGTATGCC
TGGTCTCGGCAAAACGACCCTAGCAAATAAAATATACAAGCATCCTAAAATCGGGT
ATGAATTTTTTACTCGTATTTGGGTTTATGTATCTCAATCATACAGGAGAAGAGAAT
TATTTCTCAACATCATCAGCAAATTCACTCGAAATACGAAACAATATCATGGGATGT
GTGAGGAGGATTTGGCTGATGAAATACAAGAATTCTTGGGAAAGGGAGGAAAATA
CTTGGTTGTCTTGGATGATGTATGGTCGGATGAAGCTTGGGAACGTATCAAGATA
GCTTTCCCAAATAACAACAAACCGAATCGAGTATTGTTGACCACTCGAGATTCCAA
AGTGGCTAAACAATGCACTCCCATACCTCATGATTTAAAATTTCTGAGTGAAGATG
AAAGTTGGATATTACTGGAGAAGAAGGTTTTTCACAAGGATAAATGTCCTCCTGAA
TTGGTGGTACCATCTGGGAAGAGCATAGCAAAAAAATGTAAGGGACTACCCCTTG
CGATTGTTGTTATCGCAGGAGCACTAATAGGGAAAGGCAAGACACCAAGAGAGTG
GAAACAAGTGGATGATAGTGTGAGTGAACACCTCATAAATAGAGACCACCCTGAG
AATTGTAACAAACTGGTGCAAATGAGTTATGATCGTTTACCTTACGACTTGAAAGC
GTGCTTTTTATATTGTAGTGCATTTCCCGGAGGCTTTCAAATCCCTGCTTGGAAGT
TAATCCGTTTGTGGATTGCAGAAGGTTTCATACAGTATAAAGGCCACTTATCTCTT
GAGTGTAAAGGAGAGGACAACTTGAATGATCTCATCAACAGGAATCTAGTGATGG
TAATGGAAAGAACGTCTGATGGTCAAATCAAAACATGTCGTCTTCATGACATGTTG
CACGAGTTTTGCAGGCAAGAAGCTATGAAGGAAGAAAATCTTTTCCAAGAAATAAA
GCTAGGATCTGAGCAATATTTTCCTGGAAAACGAGAACTATCCACGTACCGTCGC
TTATGCATTCATTCCTCAGTTTTGGATTTTATCTCTACGAAACCTTCAGCTGAACAT
GTGAGGTCATTCTTATCTTTTTCTTCAAAAAAGATTGAGATGCCATCTGCTGACATC
CCAACCATACCAAAAGGCTTTCCGTTGCTAAGAGTTTTGGATGTAGAATCCATCAA
CTTCAGTCGCTTTTCCAAGGAGTTTTACCAGTTATATCATTTGAGGTATGTTGCTTT
TTCATCTGACTCAATCAAGATTCTTCCTAAACTCATGGGAGAACTTTGGAACATCC
AAACCATCATAATTAACACGCAACAACGCACTCTTGATATTCAAGCAAATATATGG
AATATGGAACGACTAAGGCATCTGCACACTAACTCTTCTGCTAAATTGCCTGTCCC
TGTTGCTCCAAAAAATAGTAAAGTTACATTGGTAAATCAAAGCCTGCAAACTCTCT
CCACAATAGCTCCCGAAAGCTGCACTGAAGAAGTGTTTGCAAGGACTCCAAACCT
GAAAAAGCTGGGCATCCGTGGGAAAATAGCTGTGCTTCTTGATAATAAGTCTGCT
GTGTCGTTAAAAAATGTGAAGAGGCTAGAATACCTTGAAAACTTGAAGCTGATAAA
TGATAGTAGCATTCAAACAGGAAAGTTACGCCTTCCACCTGCATACATATTTCCAA
CAAAGTTGAGGAAGCTGACTTTATTAGACACCTGGTTGGAGTGGAAAGATATGTCT
ATATTGGGTCAGTTGGAACACCTTGAAGTCCTGAAGATGAAAGAAAATGGGTTTAC
GGGAGAGTCCTGGGAGTCTACTGGTGGTTTTTGTTCCCTACTGGTGTTGTGGATT
GAAAGGACAAACTTAGTTACTTGGAAAGCATCAGCTGATGACTTTCCAAGACTTAA
GCATCTTGTTCTCATCTGCTGTGATTACCTTAAGGAAGTCCCCATCGCCCTAGCTG
ATATACGCAGTTTCCAAGTCATGATGTTGCAAAATTCCACCAAAACAGCAGCAATA
TCTGCACGTCAAATACAAGCCAAAAAAGACAATCAAACTCAACAAGGGACTAAAAA
CATTGCCTTCAAGCTTTCCATATTCCCTCCTGATCTCTGA
SEQ ID NO: 41; Solanum tuberosum StNRC2, amino acid; NCBI Reference
Sequence: XP_006359790.1
MANVAVEFLVENLMQLLRDNAELISGVKEAAESLLQDLNDFNAFLKQAAKCHINENEV
LRELVKKIRTVVNSAEDAIDKFVIEAKLHKDKGMTRVLDLPHYKRVREVAGEIKAIRNKV
KEIRQNDAIGLQALQDDDSSARGFEERKPPVVEEDDVVGFDEEADIVIKRLLGESNRL
EVVPVVGMPGLGKTTLANKIYKHPKIGYEFFTRIWVYVSQSYRRRELFLNIISKFTRNTK
QYHGMCEEDLADEIQEFLGKGGKYLVVLDDVWSDEAWERIKIAFPNNNKPNRVLLTT
RDSKVAKQCTPIPHDLKFLSEDESWILLEKKVFHKDKCPPELVVPSGKSIAKKCKGL
PLAIVVIAGALIGKGKTPREWKQVDDSVSEHLINRDHPENCNKLVQMSYDRLPYDLK
ACFLYCSAFPGGFQIPAWKLIRLWIAEGFIQYKGHLSLECKGEDNLNDLINRNLVMVME
RTSDGQIKTCRLHDMLHEFCRQEAMKEENLFQEIKLGSEQYFPGKRELSTYRRLCIHS
SVLDFISTKPSAEHVRSFLSFSSKKIEMPSADIPTIPKGFPLLRVLDVESINFSRFSKEFY
QLYHLRYVAFSSDSIKILPKLMGELWNIQTIIINTQQRTLDIQANIWNMERLRHLHTNSS
AKLPVPVAPKNSKVTLVNQSLQTLSTIAPESCTEEVFARTPNLKKLGIRGKIAVLLDNKS
AVSLKNVKRLEYLENLKLINDSSIQTGKLRLPPAYIFPTKLRKLTLLDTWLEWKDMSILG
QLEHLEVLKMKENGFTGESWESTGGFCSLLVLWIERTNLVTWKASADDFPRLKHLVLI
CCDYLKEVPIALADIRSFQVMMLQNSTKTAAISARQIQAKKDNQTQQGTKNIAFKLSIFP
PDL
SEQ ID NO: 42; Solanum tuberosum StNRC3, cDNA; NCBI Reference Sequence:
XM_006362512.2
ATGGCGGATGTAGCAGTAAAGTTTTTAGTAGAAAATTTGATGCAATTACTAATCGA
CAACGCCGATTTGATTCTCGGAATCAAAGGCGAAGTTGAAAATCTACTCCGAGAT
CTCAATGACTTCAATGCTTTCCTCAAACAAGCTGCTAAATCCCGCAGGGAAAATGA
GGTTTTGAAAGAAATGGTGAAGAAAATCAGAAAAGTGGTGAATGATGCTGAGGAT
TCAATTGATAAATTTGTGATTGAAGCTAAGAGACATGATGATAAAAACAAATTTGCT
CAATGGTTTCATATTACTCATGTGGCTAGAGCAAAAGGGGTAGCAGATGAGATCA
AAAGTATAAAGGAAAGAGTGAAGGAAATTAGAGAAAATGACGCTTATGGCCTTCAA
GCGATAACTTTAGATGATAATTTCAATAGAGGTGATGAAGAGAGGAAGGCCCCTG
TAGTTGAGGAAGATGATGTGGTTGGTTTTGATGATGAAGCAAAAATTGTAATTGAT
CGTCTCATTGGAGGATCAGATTATGTTGAGGTTGTGCCAGTTGTTGGTATGCCTG
GTCTTGGTAAAACAACTTTGGCATATAAGATTTACAAGGATCCGAAGGTTGAGTAT
GAGTTCTTCACCCGCGTTTGGGTATATGTCTCTCAAACGTTCAAGAGAAGGGAAAT
ATTTCTCAACATTATCAGCAAGTTCACTCGAAACACCAAACAATATCATGATACACC
AGAGGATGACTTAGCAAATGAAGTGAAGGAGCTTCTTGGAAAAGGTGGAAAATAT
CTTATTGTTTTGGATGATGTGTGGACGATGGAAGCTTGGGATCGTATCAAAATTGC
TTTCCCTAATAATGGTAAACGGAATAGAGTGTTGATGACCACGAGAGAATCAAATG
TGGCTAAGTGTTGTAATGATAAACCTCATGATCTTAAGTTTTTAACTGAAGATGAAA
GTTGGGAGCTACTTGAGAAGAAGGTTTTTCACAAGGAAAAGTGTCCACCTGAGTT
AGAATTGCCCGGGAAAAGTATAGCTGAAAAATGTATGGGCTTACCTCTTGCAATAG
TGGTTATCGCCGGAGCTTTGATAGGCAAAGGAAAGACAACAAGGGAGTGGGAGT
TGGTGGCTGCCAGTGTCCGTGAGCACCTCATAAATCGAGATCCAGAGAACTGCAA
GAAATTGGTTCAAATGAGTTATGATCGTTTGCCTTATGACCTAAAGGCGTGTTTTTT
ATACTGTGGGGCATTTCCAGGAGGTTCTCAAATTCCTGCTAAGAAGTTGATTCGTT
TATGGATCGCGGAAGGATTCATACAATACCAAGGACCATTGGCTCTAGAGGATGT
AGCAGAGGACCACTTGAATGATCTTGTCAATAGGAACTTAGTGATGGTAATGCAAA
GGAGTTGCAGTGGTCAAATCAAAATATGTCGCGTTCATGACATGTTGCATGAGTTC
TGCAGACACGAGGCTATGACAGAGGAGGATCTTTTTCAAGAAATCAAACAAGGGC
AAGAACGATCTTTTCCAGGGAAACAAGAGTTAGCCACTTATCGTCGTTTGTGTATT
CATTCCGGGGTTCCTGAATTCTTATCAACAAAGCCCTCCGGTGAGCATGTCAGGT
CATTCTTATGTGTTGGCTCGAAGAAAATTGACATGCCTCCAAATGAAATACCATCC
ATCCCGAAAGCCTTTCCATTGCTTAGGGTACTAGATGCTGAATCAATCAAGTTCAA
TCGATTTTCTCGAGAGTTCTTCAAATTATTTCATTTGAGGTACATTGCTCTCTCAAC
TGACAAGATTAAGACCATTCCCGTGGATTTTGGGAATCTTTGGAACGTACAAACAC
TTATCGTTGAGACGCAAGAGGCTACTCTTGACATCAAAGCAGACATTTGGAATATG
ACAAGATTAAGGCATGTGTGCACAAACGCCTCTGCTACATTGCCTTCCACTAAGC
GCCCCAAGAGCAGCAAGGGCAACTTGGTGAATCGTTGCTTACAAACACTTTCAAC
TATAGCACCTGAATGTTGCACAGCAGAAGTTTTTACTAGAACTCCTAATCTCAAGA
AATTAGGCGTTCGTGGGAAAATAGATGCACTTTTAGAAACCAGTAAAGACGGGTC
TGGTTCTGTCTTGTTCAGTAACATAGGTAAGTTAGCTTGCCTCGAATATCTGAAGT
TGGTAAATGATACTCGTATAAGTAGTAAACCGCTACACCTTCCTCCAGCATATATTT
TCCCTCAGAAGCTAAAAAAGCTCTCTCTGGTAGACACTTGGTTTGAATGGAAAGAC
ATGTCTATATTAGGTCTATTACCAGACCTTGAGGTGCTGAAGTTGAAAGAAAATGC
GTTTAAAGGACAGTCGTGGGAGCAAGAGGACGGTGGTTTTCCTCGTCTGCAGGT
CTTATGGATTGAAAGGACAGATTTAACTTCTTGGAAAGCCTCATCAGGGAACTTTC
CGCGACTAAAACACCTCGCACTCATATCATGTGATAAGCTTGAGGAACTCCCAGC
TGAACTGGCTGATGTGAAAAACCTCCAGCTAATTGAACTCCAGAGTAGCAGTGAA
TCTGCAGCTAGATCTGCACGAGCGATACTAAAAAGGAATCAAGAAAAAGAACAGG
AAGGCGATAAAGGCACTGGATTCAAGCTTTCTATATTCCCTCATGATCTTGGATTG
TAA
SEQ ID NO: 43; Solanum tuberosum StNRC3, amino acid; NCBI Reference
Sequence: XP_006362574.1
MADVAVKFLVENLMQLLIDNADLILGIKGEVENLLRDLNDFNAFLKQAAKSRRENEVLK
EMVKKIRKVVNDAEDSIDKFVIEAKRHDDKNKFAQWFHITHVARAKGVADEIKSIKERV
KEIRENDAYGLQAITLDDNFNRGDEERKAPVVEEDDVVGFDDEAKIVIDRLIGGSDYVE
VVPVVGMPGLGKTTLAYKIYKDPKVEYEFFTRVWVYVSQTFKRREIFLNIISKFTRNTK
QYHDTPEDDLANEVKELLGKGGKYLIVLDDVWTMEAWDRIKIAFPNNGKRNRVLMTT
RESNVAKCCNDKPHDLKFLTEDESWELLEKKVFHKEKCPPELELPGKSIAEKCMGL
PLAIVVIAGALIGKGKTTREWELVAASVREHLINRDPENCKKLVQMSYDRLPYDLKAC
FLYCGAFPGGSQIPAKKLIRLWIAEGFIQYQGPLALEDVAEDHLNDLVNRNLVMVMQR
SCSGQIKICRVHDMLHEFCRHEAMTEEDLFQEIKQGQERSFPGKQELATYRRLCIHSG
VPEFLSTKPSGEHVRSFLCVGSKKIDMPPNEIPSIPKAFPLLRVLDAESIKFNRFSREFF
KLFHLRYIALSTDKIKTIPVDFGNLWNVQTLIVETQEATLDIKADIWNMTRLRHVCTNAS
ATLPSTKRPKSSKGNLVNRCLQTLSTIAPECCTAEVFTRTPNLKKLGVRGKIDALLETS
KDGSGSVLFSNIGKLACLEYLKLVNDTRISSKPLHLPPAYIFPQKLKKLSLVDTWFEWK
DMSILGLLPDLEVLKLKENAFKGQSWEQEDGGFPRLQVLWIERTDLTSWKASSGNFP
RLKHLALISCDKLEELPAELADVKNLQLIELQSSSESAARSARAILKRNQEKEQEGDKG
TGFKLSIFPHDLGL
SEQ ID NO: 44; Solanum tuberosum NRC2^{D315K}, amino acid.
MANVAVEFLVENLMQLLRDNAELISGVKEAAESLLQDLNDFNAFLKQAAKCHINENEV
LRELVKKIRTVVNSAEDAIDKFVIEAKLHKDKGMTRVLDLPHYKRVREVAGEIKAIRNKV
KEIRQNDAIGLQALQDDDSSARGFEERKPPVVEEDDVVGFDEEADIVIKRLLGESNRL
EVVPVVGMPGLGKTTLANKIYKHPKIGYEFFTRIWVYVSQSYRRRELFLNIISKFTRNTK
QYHGMCEEDLADEIQEFLGKGGKYLVVLDDVWSDEAWERIKIAFPNNNKPNRVLLTT
RDSKVAKQCTPIPHDLKFLSEKESWILLEKKVFHKDKCPPELVVPSGKSIAKKCKGLPL
AIVVIAGALIGKGKTPREWKQVDDSVSEHLINRDHPENCNKLVQMSYDRLPYDLKACF
LYCSAFPGGFQIPAWKLIRLWIAEGFIQYKGHLSLECKGEDNLNDLINRNLVMVMERTS
DGQIKTCRLHDMLHEFCRQEAMKEENLFQEIKLGSEQYFPGKRELSTYRRLCIHSSVL
DFISTKPSAEHVRSFLSFSSKKIEMPSADIPTIPKGFPLLRVLDVESINFSRFSKEFYQLY
HLRYVAFSSDSIKILPKLMGELWNIQTIIINTQQRTLDIQANIWNMERLRHLHTNSSAKLP
VPVAPKNSKVTLVNQSLQTLSTIAPESCTEEVFARTPNLKKLGIRGKIAVLLDNKSAVSL
KNVKRLEYLENLKLINDSSIQTGKLRLPPAYIFPTKLRKLTLLDTWLEWKDMSILGQLEH
LEVLKMKENGFTGESWESTGGFCSLLVLWIERTNLVTWKASADDFPRLKHLVLICCDY
LKEVPIALADIRSFQVMMLQNSTKTAAISARQIQAKKDNQTQQGTKNIAFKLSIFPPDL
SEQ ID NO: 45; Solanum tuberosum NRC3^{D315K}, amino acid.
MADVAVKFLVENLMQLLIDNADLILGIKGEVENLLRDLNDFNAFLKQAAKSRRENEVLK
EMVKKIRKVVNDAEDSIDKFVIEAKRHDDKNKFAQWFHITHVARAKGVADEIKSIKERV
KEIRENDAYGLQAITLDDNFNRGDEERKAPVVEEDDVVGFDDEAKIVIDRLIGGSDYVE
VVPVVGMPGLGKTTLAYKIYKDPKVEYEFFTRVWVYVSQTFKRREIFLNIISKFTRNTK
QYHDTPEDDLANEVKELLGKGGKYLIVLDDVWTMEAWDRIKIAFPNNGKRNRVLMTT
RESNVAKCCNDKPHDLKFLTEKESWELLEKKVFHKEKCPPELELPGKSIAEKCMGLPL
AIVVIAGALIGKGKTTREWELVAASVREHLINRDPENCKKLVQMSYDRLPYDLKACFLY
CGAFPGGSQIPAKKLIRLWIAEGFIQYQGPLALEDVAEDHLNDLVNRNLVMVMQRSCS
GQIKICRVHDMLHEFCRHEAMTEEDLFQEIKQGQERSFPGKQELATYRRLCIHSGVPE
FLSTKPSGEHVRSFLCVGSKKIDMPPNEIPSIPKAFPLLRVLDAESIKFNRFSREFFKLF
HLRYIALSTDKIKTIPVDFGNLWNVQTLIVETQEATLDIKADIWNMTRLRHVCTNASATL
PSTKRPKSSKGNLVNRCLQTLSTIAPECCTAEVFTRTPNLKKLGVRGKIDALLETSKDG
SGSVLFSNIGKLACLEYLKLVNDTRISSKPLHLPPAYIFPQKLKKLSLVDTWFEWKDMSI
LGLLPDLEVLKLKENAFKGQSWEQEDGGFPRLQVLWIERTDLTSWKASSGNFPRLKH
LALISCDKLEELPAELADVKNLQLIELQSSSESAARSARAILKRNQEKEQEGDKGTGFK
LSIFPHDLGL
SEQ ID NO: 46; Solanum lycopersicum, SINRC1 cDNA; NCBI Reference Sequence:
NM_001247273.
ATGGTTGATGTAGGGGTTGAATTTCTGTTAGAGAACTTGAAGCAATTGGTACTGGA
CAATGTGGAGTTAATCGGAGGAGCTAAAGATGAAATCGAGAATCTGCGTGATGAT
TTGAGTGAATTCAATGCCTTTCTCAAGCAAGCTGCAATGGTCCGCAGCGAAAACC
CAGTTCTCAAAGAACTAGTGAGGAGTATCAGAAAAGTGGTGAATCGTGCTGAAGA
TGCTGTTGATAAATTTGTAATTGAAGCTAAAGTTCATAAAGACAAAGGGTTTAAAG
GGGTTTTCGATAAACCTGGACATTATAGAAGAGTGAGGGATGCAGCTGTGGAGAT
TAAAGGTATCAGAGATAAAATGAGAGAAATTCGGCAAAATAAGGCACATGGCCTTC
AGGCTCTACTTCAAGATCATGATGATTCAATCAGCAGAGGTGGAGAAGAGAGACA
GCCTCCTGTGGTTGAGGAAGATGATGTGGTGGGCTTTGACGATGAGGCGCAGAC
GGTAATCGACCGTCTTCTTGAAGGATCAGGTGATTTAGAGGTTATTCCAGTAGTTG
GAATGCCTGGTCTTGGCAAAACTACACTAGCCACTAAGATCTTCAAGCATCCGAA
GATTGAGTACGAGTTCTTTACTAGACTTTGGCTTTACGTTTCCCAATCATACAAGA
CAAGAGAATTATATCTTAACATCATCAGTAAATTCACCGGAAACACCAAACATTGC
CGTGATATGTCTGAAAAGGATTTAGCTCTTAAGGTACAAGAGATTTTGGAAGAAGG
AGGAAAATACTTGATTGTCTTGGATGATGTCTGGTCGACAGATGCTTGGGATCGTA
TCAAGATTGCTTTCCCGAAAAATGACAAGGGCAATAGAGTATTGTTGACTACTCGA
GACCACCGTGTTGCAAGATATTGCAATAGGAGTCCACATGATTTAAAATTTCTGAC
TGATGAAGAGAGTTGGATTTTACTGGAGAAAAGAGCTTTTCACAAAGCTAAATGTC
TCCCCGAATTGGAAACAAACGGAAAAAGCATAGCCAGGAAGTGTAAAGGACTACC
CCTTGCTATTGTGGTGATTGCAGGAGCTCTAATTGGGAAAAGCAAAACAATAAAG
GAATGGGAGCAAGTGGATCAGAGTGTGGGCGAACATTTCATAAATAGAGATCAGC
CAAATAGTTGTGATAAATTGGTACGGATGAGTTATGATGTTTTGCCTTATGACTGG
AAAGCTTGCTTTTTATACTTCGGTACATTCCCCAGAGGCTATTTAATCCCTGCCAG
GAAATTGATCCGCTTATGGATCGCGGAAGGGTTTATCCAGTACAGAGGGGACTTA
TCCCCTGAGTGTAAAGCAGAGGAGTACTTGAATGAACTCGTAAATAGAAACTTAGT
GATGGTAATGCAAAGGACGGTTGATGGACAAATCAAAACTTGTCGTGTTCATGAC
ATGTTGTATGAGTTTTGCTGGCAAGAGGCTACGACAGAGGAAAATCTTTTCCATGA
AGTAAAATTCGGTGGTGAGCAATCTGTTCGTGAAGTATCCACTCATCGTCGCTTGT
GCATTCATTCCTCTGTTGTGGAGTTCATTTCTAAGAAGCCCTCTGGTGAGCATGTT
AGGTCGTTCCTATGTTTTTCTCCAGAAAAAATTGACACTCCCCCAACTGTCAGTGC
AAACATATCAAAAGCCTTTCCATTGCTAAGGGTGTTTGATACTGAATCCATCAAAAT
CAATCGCTTTTGCAAGGAGTTCTTTCAATTGTATCATCTGAGGTATATTGCTTTCTC
ATTTGACTCGATTAAAGTCATTCCGAAACATGTTGGGGAACTTTGGAACGTACAAA
CCCTCATTGTCAACACACAACAGATCAACCTTGATATTCAAGCAGACATATTGAAC
ATGCCCCGGCTGAGGCATCTGCTCACCAACACGTCTGCTAAATTGCCTGCGCTTG
CTAACCCCAAAACAAGTAAGACTACCTTGGTAAATCAAAGCCTGCAAACCCTCTCC
ACAATTGCACCAGAAAGCTGCACTGAGTATGTTCTCTCGAGGGCTCCAAACTTGA
AAAAACTGGGCATTCGTGGAAAAATAGCTAAGCTAATGGAACCAAGTCAGTCTGTA
TTGTTGAACAATGTTAAGAGGCTGCAATTTCTTGAGAACTTGAAGCTGATAAATGT
TGGTCAGATTGATCAGACACAATTACGCCTTCCTCCAGCATCTATATTTCCAACAA
AGTTGAGGAAGCTGACTTTATTAGATACCTGGTTGGAGTGGGATGATATGTCTGTA
TTGAAACAGCTGGAGAACCTTCAAGTCTTGAAGCTGAAGGACAATGCATTTAAGG
GAGAGAACTGGGAACTAAATGATGGAGGTTTTCCTTTCCTACAAGTGTTATGCATT
GAAAGGGCAAACTTAGTTTCTTGGAATGCTTCAGGTGATCACTTCCCGAGACTTAA
ACATCTTCACATATCATGTGATAAACTTGAGAAGATCCCCATTGGCCTGGCTGATA
TATGCAGCCTCCAAGTGATGGATTTGCGAAATTCCACTAAATCAGCAGCAAAATCT
GCCAGAGAGATACAAGCCAAAAAAAACAAGCTGCAACCTGCTAAATCCCAGAAGT
TCGAGCTTTCTGTATTCCCTCCTGATTCTGATGTACAGACAGCTTCTTAG
SEQ ID NO: 47; Solanum lycopersicum, SINRC1 amino acid; NCBI Reference
Sequence: NP_001234202.
MVDVGVEFLLENLKQLVLDNVELIGGAKDEIENLRDDLSEFNAFLKQAAMVRSENPVL
KELVRSIRKVVNRAEDAVDKFVIEAKVHKDKGFKGVFDKPGHYRRVRDAAVEIKGIRD
KMREIRQNKAHGLQALLQDHDDSISRGGEERQPPVVEEDDVVGFDDEAQTVIDRLLE
GSGDLEVIPVVGMPGLGKTTLATKIFKHPKIEYEFFTRLWLYVSQSYKTRELYLNIISKF
TGNTKHCRDMSEKDLALKVQEILEEGGKYLIVLDDVWSTDAWDRIKIAFPKNDKGNRV
LLTTRDHRVARYCNRSPHDLKFLTDEESWILLEKRAFHKAKCLPELETNGKSIARKC
KGLPLAIVVIAGALIGKSKTIKEWEQVDQSVGEHFINRDQPNSCDKLVRMSYDVLPYD
WKACFLYFGTFPRGYLIPARKLIRLWIAEGFIQYRGDLSPECKAEEYLNELVNRNLVMV
MQRTVDGQIKTCRVHDMLYEFCWQEATTEENLFHEVKFGGEQSVREVSTHRRLCIH
SSVVEFISKKPSGEHVRSFLCFSPEKIDTPPTVSANISKAFPLLRVFDTESIKINRFCKEF
FQLYHLRYIAFSFDSIKVIPKHVGELWNVQTLIVNTQQINLDIQADILNMPRLRHLLTNTS
AKLPALANPKTSKTTLVNQSLQTLSTIAPESCTEYVLSRAPNLKKLGIRGKIAKLMEPSQ
SVLLNNVKRLQFLENLKLINVGQIDQTQLRLPPASIFPTKLRKLTLLDTWLEWDDMSVL
KQLENLQVLKLKDNAFKGENWELNDGGFPFLQVLCIERANLVSWNASGDHFPRLKHL
HISCDKLEKIPIGLADICSLQVMDLRNSTKSAAKSAREIQAKKNKLQPAKSQKFELSVFP
PDSDVQTAS
SEQ ID NO: 48; Solanum lycopersicum, SINRC2 cDNA; NCBI Reference Sequence:
XM_004248750.
ATGGCGAACGTAGCAGTGGAATTTCTGGTTGAGAACTTGATGCAGTTGCTGCGGG
ACAACGTAGAGCTAATTAGTGGAGTTAAAGAGGCTGCTGAGAGTCTACTTCAAGA
TCTAAATGATTTCAATGCTTTTCTTAAGCAAGCTGCCAAGTGTCACATCAACGAGA
ACGAAGTCCTCAGAGAACTTGTTAAGAAAATAAGAACAGTCGTTAACTCTGCTGAA
GATGCTATTGATAAATTTGTTATTGAAGCTAAGCTACACAAGGATAAGGGTGTCAC
CAGAGTATTAGATCTTCCCCATTATAAAAGAGTCAAAGAGGTAGCTGGTGAGATCA
AAGCTATACGAAACAAAGTCAGAGAGATCCGACAGACTGATGCCATTGGACTTCA
GGCCCTTCAAGATGATGATTTATCTGCTAGAGGTTCCGAAGAAAGAAAGCCTCCA
GTGGTAGAGGAAGATGACGTGGTTGGATTTGACGAAGAAGCAGATATTGTAATCA
ACCGCCTTCTTGGAGAATCGAATCATCTAGAAGTTGTTCCAGTTGTTGGTATGCCT
GGTCTCGGCAAAACGACCCTAGCAAATAAGATTTACAAGCATCCTAAAATCGGGT
ATGAGTTTTTTACTCGTATTTGGGTTTATGTGTCTCAATCATACAGGAGAAGAGAAT
TATTTCTCAACATCATCAGCAAATTCACTCGAAATACAAAACAATATCATGGGATGT
GTGAGGAGGATTTGGCTGATGAAATACAAGAATTCTTGGGAAAGGGAGGAAAATA
CTTGGTTGTGTTGGATGACGTATGGTCGGATGAAGCTTGGGAACGTATCAAGATA
GCATTCCCAAATAACAACAAACCGAATCGAGTATTGTTGACCACTCGAGATTCCAA
AGTGGCTAAACAATGCAATCCCATACCTCATGATCTAAAATTTCTGACTGAAGATG
AAAGTTGGATATTACTGGAGAAGAAGGTTTTTCACAAGGACAAATGTCCTCCTGAA
TTGGTACTATCTGGAAAGAGCATAGCAAAAAAATGCAAGGGACTACCACTTGCGA
TTGTTGTTATTGCAGGAGCACTAATAGGGAAAGGCAAGACACCAAGAGAGTGGAA
ACAAGTGGATGATAGTGTTAGTGAACACCTCATAAATAGAGACCACCCTGAGAATT
GTAACAAACTGGTGCAAATGAGTTATGATCGTTTACCTTACGACTTGAAAGCATGC
TTTTTATATTGTAGTGCATTTCCTGGAGGCTTTCAAATCCCTGCTTGGAAGTTAATC
CGTTTGTGGATTGCGGAAGGATTCATACAGTATAAAGGCCATTTATCTCTTGAGTG
TAAAGGAGAGGACAACTTGAATGATCTCATCAATAGGAATCTAGTGATGGTAATGG
AAAGAACTTCTGATGGTCAAATCAAAACATGTCGTCTTCATGACATGTTGCACGAG
TTTTGCAGGCAAGAAGCTATGAAGGAAGAAAATCTTTTCCAAGAAATAAAGTTAGG
ATCTGAGCAATATTTTCCTGGAAAACGAGAACTATCCACCTACCGTCGCTTATGCA
TCCATTCCTCAGTTTTGGATTTTTTCTCTACGAAACCTTCAGCTGAACATGTCAGGT
CATTCTTATCTTTTTCTTCGAAAAAGATTGAGATGCCATCTGCTGACATCCCAACCA
TACCCAAAGGCTTTCCGTTGCTAAGGGTTTTGGATGTAGAATCAATCAACTTCAGT
CGCTTTTCCAGGGAGTTTTACCAGTTATATCATTTGAGGTATGTTGCTTTTTCATCT
GACTCAATCAAGATTCTTCCTAAACTCATGGGAGAACTTTGGAACATCCAAACCAT
CATAATTAACACGCAACAACGCACTCTTGATATTCAAGCAAACATATGGAATATGG
AACGACTAAGGCATCTGCACACTAACTCTTCCGCTAAGTTGCCTGTCCCTGTTGCT
CCAAAAAATAGTAAGGTTACATTGGTAAATCAAAGCCTGCAAACTCTCTCCACCAT
AGCTCCCGAAAGCTGCACCGAGGAAGTGTTTGCGAGGACTCCAAACCTGAAAAA
GCTGGGCATCCGTGGGAAAATATCAGTGCTTCTTGATAATAAGTCTGCTGCGTCG
TTAAAAAATGTGAAGAGGCTAGAATACCTTGAAAACTTGAAGCTGATAAATGATAG
TAGCATTCAAACAAGTAAGTTACGCCTTCCACCTGCATACATATTTCCAACAAAGTT
GAGGAAGCTGACTTTATTAGACACGTGGTTGGAGTGGAAAGATATGTCTATATTG
GGTCAGTTGGAACACCTTGAAGTCCTGAAGATGAAAGAAAATGGGTTTTCGGGAG
AGTCCTGGGAGTCTACTGGTGGTTTTTGTTCCCTACTGGTGTTGTGGATTGAAAG
GACAAACTTAGTTTCATGGAAAGCATCAGCTGATGATTTTCCAAGACTTAAGCATC
TCGTTCTCATCTGCTGTGATAATCTTAAGGAAGTCCCCATCGCCCTAGCTGATATA
CGCAGTTTCCAAGTCATGATGTTGCAAAATTCCACCAAAACAGCAGCAATATCTGC
ACGTCAAATACAAGCCAAAAAAGACAATCAAACTCAACAAGGGACTAAAAACATTG
CCTTCAAGCTTTCCATATTCCCTCCTGATCTCTGA
SEQ ID NO: 49; Solanum lycopersicum, SINRC2 amino acid; NCBI Reference
Sequence: XP_004248798.
MANVAVEFLVENLMQLLRDNVELISGVKEAAESLLQDLNDFNAFLKQAAKCHINENEV
LRELVKKIRTVVNSAEDAIDKFVIEAKLHKDKGVTRVLDLPHYKRVKEVAGEIKAIRNKV
REIRQTDAIGLQALQDDDLSARGSEERKPPVVEEDDVVGFDEEADIVINRLLGESNHL
EVVPVVGMPGLGKTTLANKIYKHPKIGYEFFTRIWVYVSQSYRRRELFLNIISKFTRNTK
QYHGMCEEDLADEIQEFLGKGGKYLVVLDDVWSDEAWERIKIAFPNNNKPNRVLLTT
RDSKVAKQCNPIPHDLKFLTEDESWILLEKKVFHKDKCPPELVLSGKSIAKKCKGLP
LAIVVIAGALIGKGKTPREWKQVDDSVSEHLINRDHPENCNKLVQMSYDRLPYDLKA
CFLYCSAFPGGFQIPAWKLIRLWIAEGFIQYKGHLSLECKGEDNLNDLINRNLVMVME
RTSDGQIKTCRLHDMLHEFCRQEAMKEENLFQEIKLGSEQYFPGKRELSTYRRLCIHS
SVLDFFSTKPSAEHVRSFLSFSSKKIEMPSADIPTIPKGFPLLRVLDVESINFSRFSREF
YQLYHLRYVAFSSDSIKILPKLMGELWNIQTIIINTQQRTLDIQANIWNMERLRHLHTNS
SAKLPVPVAPKNSKVTLVNQSLQTLSTIAPESCTEEVFARTPNLKKLGIRGKISVLLDNK
SAASLKNVKRLEYLENLKLINDSSIQTSKLRLPPAYIFPTKLRKLTLLDTWLEWKDMSIL
GQLEHLEVLKMKENGFSGESWESTGGFCSLLVLWIERTNLVSWKASADDFPRLKHLV
LICCDNLKEVPIALADIRSFQVMMLQNSTKTAAISARQIQAKKDNQTQQGTKNIAFKLSI
FPPDL
SEQ ID NO: 50; Solanum lycopersicum, SINRC3, cDNA; NCBI Reference
Sequence: XM_004238900.4.
ATGGCGGATGTAGCAGTAAAGTTCTTATTAGAAAATTTGACGCAGTTACTAATCGA
CAACGCCGATTTGATTCTCGGAATCCAAGGCGAAGTTGAAAATCTACTCACAGATC
TCAATTACTTTAATGCTTTCCTCAAAGAAGCTGCTAAATCCCGCAGGGAAAATGAG
GTTTTGAAAGAATTGGTGAAGAAAATCAGAAAAGTTGTGAACGATGCTGAAGATTC
GATTGATAAATTTGTGGTTGAAGCTAAGAGACATGATGATAAAAACAAATTTGCTC
AATGGTTTCATATTACTCATGTGGCTAGAGCCAAAGGGGTAGCGGATGAGATCAA
AAGTATAAGGGAAAGAGTGAAGGAAATTAGAGATAATGACGCTTATGGCCTTCAA
GCTATAACTTTGGATGATAATTTCAACAGAGGTGACGAAGAGAGGAAGGCCCCTG
TAGTTGAGGAAGATGATGTGGTTGGTTTTGATGATGAAGCAAAAACTGTAATTGAT
CGTCTCATCGGAGGATCAGACTATGTTGAGGTTGTGCCAGTTGTTGGTATGCCTG
GTCTTGGTAAAACAACTTTGGCATATAAGATTTACAAGGATCCAAAGGTTGAGTAT
GAGTTCTTCACCCGCGTTTGGGTATATGTCTCTCAAACATTCAAGAGAAGGGAAAT
ATTTCTCAACATTATCAGCAAGTTTACTCGAAACACCAAACAATATGATGATACACC
AGAGGATGACTTAGCAAATGAAGTGAAGGAGCTTCTTGGAAAAGGTGGAAAATAT
CTTATTGTTTTGGATGATGTGTGGACGATGGAAGCTTGGGATCGTATCAAAATTGC
TTTCCCTAATAATGGTAAACGAAATAGAGTGTTGATGACCACGAGACAATCAAATG
TGGCGAAGCGTTGTAATGATAAACCTCATGATCTTAAGTTTTTAACAAAAGATGAA
AGTTGGGAGCTACTTGAGAAGAAGGTTTTTCACAAGGAAAAGTGTCCACCTGAGT
TAGAATTACCCGGGATAAGTATAGCCGAAAAATGTATGGGCTTACCTCTAGCTATA
GTGGTTATTGCCGGAGCTTTGATAGGCAAAGGAAAGACAACAAGGGAGTGGGAG
TTGGTGGCTGCCAGTGTCGGTGAGCACCTCATAAATCGAGATCCAGAGAACTGCA
AGAAATTGGTTCAAATGAGTTATGATCGTTTGCCTTATGACCTAAAGGCGTGTTTTT
TATACTGTGGGGCATTTCCAGGAGGTTCTCAAATTCCAGCTAAGAAGTTGATTCGT
TTATGGATCGCGGAAGGATTCATACAATACCAAGGACCATTGGCTCTGGAGGATG
TAGCAGAGGACCACTTAAATGATCTTGTCAATAGGAACTTAGTGATGGTAACGCAA
AGGAGTTGCAGTGGTCAAATCAAAACATGTCGTGTTCATGACATGTTGCATGAGTT
CTGCAGACACGAGGCCATGATGGAGGAGAATCTTTTTCAAGAAATCAAACAAGGG
CAAGAACGATCTTTTCCAGGGAAACAAGAGTTAGCCACTTATCGTCGTTTGTGTAT
TCAATCCTTGATTCCTGAATTCTTATCAATGAAGCCCTCTGGTGAGCATGTAAGGT
CATTCTTATGTGTTGGCTCGAAGAAAATTGACATGCCTCCAAATGAAATACCGTCC
ATCCCGAAAGCCTTTCCATTGCTTAGGGTACTAGATGCTGAATCAATCAAGTTCAG
TCGATTTTCTCGTGAGTTCTTCAAATTATTTCATTTGAGGTACATTGCTCTCTCAAC
TGACAAGATTAAGACCATTCCCGCGGATTTTGGGAATCTTTGGAACATACAAACAC
TTATCGTTGAGACGCAACAGGCTACTCTTGACATCAAGGCAGACATTTGGAATATG
ACAAGACTAAGGCATGTGTGCACAAACGCCTCAGCTACATTGCCTTCTACTAAGC
GCCCCAAGAGCAGCAAGGACAACTTGGTGAATCGATGCTTACAAACACTTTCAAC
TATAGCACCAGAGTGTTGCACAGCAGAGGTTTTTACTAGAACTCCTAATCTCAAGA
AATTAGGCGTTCGTGGGAAAATAGATGCACTTTTAGAATCCAGTAAAGACGGGTCT
GGTTCTGGCTTGTTCAGTAACATAGGTAAGTTAGGTTGCCTCGAATATCTGAAGTT
AGTAAATGATACTCGTCTAAGTAGTAAACCTCTACACCTTCCTCCTGCATATATTTT
CCCTCAGAAGCTAAAAAAGCTCTCTCTGGTAGACACTTGGTTTGAATGGAAAGATA
TGTCTATATTAGGTCTATTACCAGAACTTGAGGTGCTGAAGTTGAAAGAAAATGCG
TTTAAAGGACAGTCGTGGGAGCAAGAGGACGGTGGTTTTCCTCGTCTGCAGGTCT
TATGGATTGAAAGGACTGATCTAACTTCTTGGAAAGCCTCATCAGGGAACTTTCCG
CGACTAAAACACCTCGCACTCATATCATGTGATAAGCTTGAGGAACTCCCAGCTG
AACTGGCTGATGTGAAAAACCTCCAGCTAATCGAACTCCAGAGTAGCAGTGAGTC
TGCAGCTAGATCTGCACGAGCGATACTAAAAAGGAATCAAGAAAAAGAACAGGAT
GGCGATAAAGGCACTGGATTCAAGCTTTCTATATTCCCTCATGATCTTGGATTGTA
A
SEQ ID NO: 51; Solanum lycopersicum, SINRC3, amino acid; NCBI Reference
Sequence: XP_004238948.
MADVAVKFLLENLTQLLIDNADLILGIQGEVENLLTDLNYFNAFLKEAAKSRRENEVLKE
LVKKIRKVVNDAEDSIDKFVVEAKRHDDKNKFAQWFHITHVARAKGVADEIKSIRERVK
EIRDNDAYGLQAITLDDNFNRGDEERKAPVVEEDDVVGFDDEAKTVIDRLIGGSDYVE
VVPVVGMPGLGKTTLAYKIYKDPKVEYEFFTRVWVYVSQTFKRREIFLNIISKFTRNTK
QYDDTPEDDLANEVKELLGKGGKYLIVLDDVWTMEAWDRIKIAFPNNGKRNRVLMTT
RQSNVAKRCNDKPHDLKFLTKDESWELLEKKVFHKEKCPPELELPGISIAEKCMGLP
LAIVVIAGALIGKGKTTREWELVAASVGEHLINRDPENCKKLVQMSYDRLPYDLKACF
LYCGAFPGGSQIPAKKLIRLWIAEGFIQYQGPLALEDVAEDHLNDLVNRNLVMVTQRS
CSGQIKTCRVHDMLHEFCRHEAMMEENLFQEIKQGQERSFPGKQELATYRRLCIQSLI
PEFLSMKPSGEHVRSFLCVGSKKIDMPPNEIPSIPKAFPLLRVLDAESIKFSRFSREFFK
LFHLRYIALSTDKIKTIPADFGNLWNIQTLIVETQQATLDIKADIWNMTRLRHVCTNASAT
LPSTKRPKSSKDNLVNRCLQTLSTIAPECCTAEVFTRTPNLKKLGVRGKIDALLESSKD
GSGSGLFSNIGKLGCLEYLKLVNDTRLSSKPLHLPPAYIFPQKLKKLSLVDTWFEWKD
MSILGLLPELEVLKLKENAFKGQSWEQEDGGFPRLQVLWIERTDLTSWKASSGNFPR
LKHLALISCDKLEELPAELADVKNLQLIELQSSSESAARSARAILKRNQEKEQDGDKGT
GFKLSIFPHDLGL
SEQ ID NO: 52; Solanum lycopersicum NRC1^{D316K}, amino acid.
MVDVGVEFLLENLKQLVLDNVELIGGAKDEIENLRDDLSEFNAFLKQAAMVRSENPVL
KELVRSIRKVVNRAEDAVDKFVIEAKVHKDKGFKGVFDKPGHYRRVRDAAVEIKGIRD
KMREIRQNKAHGLQALLQDHDDSISRGGEERQPPVVEEDDVVGFDDEAQTVIDRLLE
GSGDLEVIPVVGMPGLGKTTLATKIFKHPKIEYEFFTRLWLYVSQSYKTRELYLNIISKF
TGNTKHCRDMSEKDLALKVQEILEEGGKYLIVLDDVWSTDAWDRIKIAFPKNDKGNRV
LLTTRDHRVARYCNRSPHDLKFLTKEESWILLEKRAFHKAKCLPELETNGKSIARKCK
GLPLAIVVIAGALIGKSKTIKEWEQVDQSVGEHFINRDQPNSCDKLVRMSYDVLPYDW
KACFLYFGTFPRGYLIPARKLIRLWIAEGFIQYRGDLSPECKAEEYLNELVNRNLVMVM
QRTVDGQIKTCRVHDMLYEFCWQEATTEENLFHEVKFGGEQSVREVSTHRRLCIHSS
VVEFISKKPSGEHVRSFLCFSPEKIDTPPTVSANISKAFPLLRVFDTESIKINRFCKEFFQ
LYHLRYIAFSFDSIKVIPKHVGELWNVQTLIVNTQQINLDIQADILNMPRLRHLLTNTSAK
LPALANPKTSKTTLVNQSLQTLSTIAPESCTEYVLSRAPNLKKLGIRGKIAKLMEPSQSV
LLNNVKRLQFLENLKLINVGQIDQTQLRLPPASIFPTKLRKLTLLDTWLEWDDMSVLKQ
LENLQVLKLKDNAFKGENWELNDGGFPFLQVLCIERANLVSWNASGDHFPRLKHLHIS
CDKLEKIPIGLADICSLQVMDLRNSTKSAAKSAREIQAKKNKLQPAKSQKFELSVFPPD
SDVQTAS
SEQ ID NO: 53; Solanum lycopersicum NRC2^{D315K}, amino acid
MANVAVEFLVENLMQLLRDNVELISGVKEAAESLLQDLNDFNAFLKQAAKCHINENEV
LRELVKKIRTVVNSAEDAIDKFVIEAKLHKDKGVTRVLDLPHYKRVKEVAGEIKAIRNKV
REIRQTDAIGLQALQDDDLSARGSEERKPPVVEEDDVVGFDEEADIVINRLLGESNHL
EVVPVVGMPGLGKTTLANKIYKHPKIGYEFFTRIWVYVSQSYRRRELFLNIISKFTRNTK
QYHGMCEEDLADEIQEFLGKGGKYLVVLDDVWSDEAWERIKIAFPNNNKPNRVLLTT
RDSKVAKQCNPIPHDLKFLTEKESWILLEKKVFHKDKCPPELVLSGKSIAKKCKGLPLAI
VVIAGALIGKGKTPREWKQVDDSVSEHLINRDHPENCNKLVQMSYDRLPYDLKACFLY
CSAFPGGFQIPAWKLIRLWIAEGFIQYKGHLSLECKGEDNLNDLINRNLVMVMERTSD
GQIKTCRLHDMLHEFCRQEAMKEENLFQEIKLGSEQYFPGKRELSTYRRLCIHSSVLD
FFSTKPSAEHVRSFLSFSSKKIEMPSADIPTIPKGFPLLRVLDVESINFSRFSREFYQLY
HLRYVAFSSDSIKILPKLMGELWNIQTIIINTQQRTLDIQANIWNMERLRHLHTNSSAKLP
VPVAPKNSKVTLVNQSLQTLSTIAPESCTEEVFARTPNLKKLGIRGKISVLLDNKSAASL
KNVKRLEYLENLKLINDSSIQTSKLRLPPAYIFPTKLRKLTLLDTWLEWKDMSILGQLEH
LEVLKMKENGFSGESWESTGGFCSLLVLWIERTNLVSWKASADDFPRLKHLVLICCD
NLKEVPIALADIRSFQVMMLQNSTKTAAISARQIQAKKDNQTQQGTKNIAFKLSIFPPDL
SEQ ID NO: 54; Solanum lycopersicum NRC3^{D315K}, amino acid
MADVAVKFLLENLTQLLIDNADLILGIQGEVENLLTDLNYFNAFLKEAAKSRRENEVLKE
LVKKIRKVVNDAEDSIDKFVVEAKRHDDKNKFAQWFHITHVARAKGVADEIKSIRERVK
EIRDNDAYGLQAITLDDNFNRGDEERKAPVVEEDDVVGFDDEAKTVIDRLIGGSDYVE
VVPVVGMPGLGKTTLAYKIYKDPKVEYEFFTRVWVYVSQTFKRREIFLNIISKFTRNTK
QYDDTPEDDLANEVKELLGKGGKYLIVLDDVWTMEAWDRIKIAFPNNGKRNRVLMTT
RQSNVAKRCNDKPHDLKFLTKDESWELLEKKVFHKEKCPPELELPGISIAEKCMGLPL
AIVVIAGALIGKGKTTREWELVAASVGEHLINRDPENCKKLVQMSYDRLPYDLKACFLY
CGAFPGGSQIPAKKLIRLWIAEGFIQYQGPLALEDVAEDHLNDLVNRNLVMVTQRSCS
GQIKTCRVHDMLHEFCRHEAMMEENLFQEIKQGQERSFPGKQELATYRRLCIQSLIPE
FLSMKPSGEHVRSFLCVGSKKIDMPPNEIPSIPKAFPLLRVLDAESIKFSRFSREFFKLF
HLRYIALSTDKIKTIPADFGNLWNIQTLIVETQQATLDIKADIWNMTRLRHVCTNASATLP
STKRPKSSKDNLVNRCLQTLSTIAPECCTAEVFTRTPNLKKLGVRGKIDALLESSKDGS
GSGLFSNIGKLGCLEYLKLVNDTRLSSKPLHLPPAYIFPQKLKKLSLVDTWFEWKDMSI
LGLLPELEVLKLKENAFKGQSWEQEDGGFPRLQVLWIERTDLTSWKASSGNFPRLKH
LALISCDKLEELPAELADVKNLQLIELQSSSESAARSARAILKRNQEKEQDGDKGTGFK
LSIFPHDLGL
SEQ ID NO: 55; Solanum tuberosum NRC2^{S317K}, amino acid.
MANVAVEFLVENLMQLLRDNAELISGVKEAAESLLQDLNDFNAFLKQAAKCHINENEV
LRELVKKIRTVVNSAEDAIDKFVIEAKLHKDKGMTRVLDLPHYKRVREVAGEIKAIRNKV
KEIRQNDAIGLQALQDDDSSARGFEERKPPVVEEDDVVGFDEEADIVIKRLLGESNRL
EVVPVVGMPGLGKTTLANKIYKHPKIGYEFFTRIWVYVSQSYRRRELFLNIISKFTRNTK
QYHGMCEEDLADEIQEFLGKGGKYLVVLDDVWSDEAWERIKIAFPNNNKPNRVLLTT
RDSKVAKQCTPIPHDLKFLSEDEKWILLEKKVFHKDKCPPELVVPSGKSIAKKCKGLPL
AIVVIAGALIGKGKTPREWKQVDDSVSEHLINRDHPENCNKLVQMSYDRLPYDLKACF
LYCSAFPGGFQIPAWKLIRLWIAEGFIQYKGHLSLECKGEDNLNDLINRNLVMVMERTS
DGQIKTCRLHDMLHEFCRQEAMKEENLFQEIKLGSEQYFPGKRELSTYRRLCIHSSVL
DFISTKPSAEHVRSFLSFSSKKIEMPSADIPTIPKGFPLLRVLDVESINFSRFSKEFYQLY
HLRYVAFSSDSIKILPKLMGELWNIQTIIINTQQRTLDIQANIWNMERLRHLHTNSSAKLP
VPVAPKNSKVTLVNQSLQTLSTIAPESCTEEVFARTPNLKKLGIRGKIAVLLDNKSAVSL
KNVKRLEYLENLKLINDSSIQTGKLRLPPAYIFPTKLRKLTLLDTWLEWKDMSILGQLEH
LEVLKMKENGFTGESWESTGGFCSLLVLWIERTNLVTWKASADDFPRLKHLVLICCDY
LKEVPIALADIRSFQVMMLQNSTKTAAISARQIQAKKDNQTQQGTKNIAFKLSIFPPDL
SEQ ID NO: 56; Solanum tuberosum NRC3^{S317K}, amino acid.
MADVAVKFLVENLMQLLIDNADLILGIKGEVENLLRDLNDFNAFLKQAAKSRRENEVLK
EMVKKIRKVVNDAEDSIDKFVIEAKRHDDKNKFAQWFHITHVARAKGVADEIKSIKERV
KEIRENDAYGLQAITLDDNFNRGDEERKAPVVEEDDVVGFDDEAKIVIDRLIGGSDYVE
VVPVVGMPGLGKTTLAYKIYKDPKVEYEFFTRVWVYVSQTFKRREIFLNIISKFTRNTK
QYHDTPEDDLANEVKELLGKGGKYLIVLDDVWTMEAWDRIKIAFPNNGKRNRVLMTT
RESNVAKCCNDKPHDLKFLTEDEKWELLEKKVFHKEKCPPELELPGKSIAEKCMGLPL
AIVVIAGALIGKGKTTREWELVAASVREHLINRDPENCKKLVQMSYDRLPYDLKACFLY
CGAFPGGSQIPAKKLIRLWIAEGFIQYQGPLALEDVAEDHLNDLVNRNLVMVMQRSCS
GQIKICRVHDMLHEFCRHEAMTEEDLFQEIKQGQERSFPGKQELATYRRLCIHSGVPE
FLSTKPSGEHVRSFLCVGSKKIDMPPNEIPSIPKAFPLLRVLDAESIKFNRFSREFFKLF
HLRYIALSTDKIKTIPVDFGNLWNVQTLIVETQEATLDIKADIWNMTRLRHVCTNASATL
PSTKRPKSSKGNLVNRCLQTLSTIAPECCTAEVFTRTPNLKKLGVRGKIDALLETSKDG
SGSVLFSNIGKLACLEYLKLVNDTRISSKPLHLPPAYIFPQKLKKLSLVDTWFEWKDMSI
LGLLPDLEVLKLKENAFKGQSWEQEDGGFPRLQVLWIERTDLTSWKASSGNFPRLKH
LALISCDKLEELPAELADVKNLQLIELQSSSESAARSARAILKRNQEKEQEGDKGTGFK
LSIFPHDLGL
SEQ ID NO: 57; Solanum lycopersicum NRC1^{E317K}, amino acid.
MVDVGVEFLLENLKQLVLDNVELIGGAKDEIENLRDDLSEFNAFLKQAAMVRSENPVL
KELVRSIRKVVNRAEDAVDKFVIEAKVHKDKGFKGVFDKPGHYRRVRDAAVEIKGIRD
KMREIRQNKAHGLQALLQDHDDSISRGGEERQPPVVEEDDVVGFDDEAQTVIDRLLE
GSGDLEVIPVVGMPGLGKTTLATKIFKHPKIEYEFFTRLWLYVSQSYKTRELYLNIISKF
TGNTKHCRDMSEKDLALKVQEILEEGGKYLIVLDDVWSTDAWDRIKIAFPKNDKGNRV
LLTTRDHRVARYCNRSPHDLKFLTDKESWILLEKRAFHKAKCLPELETNGKSIARKCK
GLPLAIVVIAGALIGKSKTIKEWEQVDQSVGEHFINRDQPNSCDKLVRMSYDVLPYDW
KACFLYFGTFPRGYLIPARKLIRLWIAEGFIQYRGDLSPECKAEEYLNELVNRNLVMVM
QRTVDGQIKTCRVHDMLYEFCWQEATTEENLFHEVKFGGEQSVREVSTHRRLCIHSS
VVEFISKKPSGEHVRSFLCFSPEKIDTPPTVSANISKAFPLLRVFDTESIKINRFCKEFFQ
LYHLRYIAFSFDSIKVIPKHVGELWNVQTLIVNTQQINLDIQADILNMPRLRHLLTNTSAK
LPALANPKTSKTTLVNQSLQTLSTIAPESCTEYVLSRAPNLKKLGIRGKIAKLMEPSQSV
LLNNVKRLQFLENLKLINVGQIDQTQLRLPPASIFPTKLRKLTLLDTWLEWDDMSVLKQ
LENLQVLKLKDNAFKGENWELNDGGFPFLQVLCIERANLVSWNASGDHFPRLKHLHIS
CDKLEKIPIGLADICSLQVMDLRNSTKSAAKSAREIQAKKNKLQPAKSQKFELSVFPPD
SDVQTAS
SEQ ID NO: 58; Solanum lycopersicum NRC2^{S317K}, amino acid
MANVAVEFLVENLMQLLRDNVELISGVKEAAESLLQDLNDFNAFLKQAAKCHINENEV
LRELVKKIRTVVNSAEDAIDKFVIEAKLHKDKGVTRVLDLPHYKRVKEVAGEIKAIRNKV
REIRQTDAIGLQALQDDDLSARGSEERKPPVVEEDDVVGFDEEADIVINRLLGESNHL
EVVPVVGMPGLGKTTLANKIYKHPKIGYEFFTRIWVYVSQSYRRRELFLNIISKFTRNTK
QYHGMCEEDLADEIQEFLGKGGKYLVVLDDVWSDEAWERIKIAFPNNNKPNRVLLTT
RDSKVAKQCNPIPHDLKFLTEDEKWILLEKKVFHKDKCPPELVLSGKSIAKKCKGLPLA
IVVIAGALIGKGKTPREWKQVDDSVSEHLINRDHPENCNKLVQMSYDRLPYDLKACFL
YCSAFPGGFQIPAWKLIRLWIAEGFIQYKGHLSLECKGEDNLNDLINRNLVMVMERTS
DGQIKTCRLHDMLHEFCRQEAMKEENLFQEIKLGSEQYFPGKRELSTYRRLCIHSSVL
DFFSTKPSAEHVRSFLSFSSKKIEMPSADIPTIPKGFPLLRVLDVESINFSRFSREFYQL
YHLRYVAFSSDSIKILPKLMGELWNIQTIIINTQQRTLDIQANIWNMERLRHLHTNSSAKL
PVPVAPKNSKVTLVNQSLQTLSTIAPESCTEEVFARTPNLKKLGIRGKISVLLDNKSAAS
LKNVKRLEYLENLKLINDSSIQTSKLRLPPAYIFPTKLRKLTLLDTWLEWKDMSILGQLE
HLEVLKMKENGFSGESWESTGGFCSLLVLWIERTNLVSWKASADDFPRLKHLVLICC
DNLKEVPIALADIRSFQVMMLQNSTKTAAISARQIQAKKDNQTQQGTKNIAFKLSIFPPD
L
SEQ ID NO: 59; Solanum lycopersicum NRC3^{S317K}, amino acid
MADVAVKFLLENLTQLLIDNADLILGIQGEVENLLTDLNYFNAFLKEAAKSRRENEVLKE
LVKKIRKVVNDAEDSIDKFVVEAKRHDDKNKFAQWFHITHVARAKGVADEIKSIRERVK
EIRDNDAYGLQAITLDDNFNRGDEERKAPVVEEDDVVGFDDEAKTVIDRLIGGSDYVE
VVPVVGMPGLGKTTLAYKIYKDPKVEYEFFTRVWVYVSQTFKRREIFLNIISKFTRNTK
QYDDTPEDDLANEVKELLGKGGKYLIVLDDVWTMEAWDRIKIAFPNNGKRNRVLMTT
RQSNVAKRCNDKPHDLKFLTKDEKWELLEKKVFHKEKCPPELELPGISIAEKCMGLPL
AIVVIAGALIGKGKTTREWELVAASVGEHLINRDPENCKKLVQMSYDRLPYDLKACFLY
CGAFPGGSQIPAKKLIRLWIAEGFIQYQGPLALEDVAEDHLNDLVNRNLVMVTQRSCS
GQIKTCRVHDMLHEFCRHEAMMEENLFQEIKQGQERSFPGKQELATYRRLCIQSLIPE
FLSMKPSGEHVRSFLCVGSKKIDMPPNEIPSIPKAFPLLRVLDAESIKFSRFSREFFKLF
HLRYIALSTDKIKTIPADFGNLWNIQTLIVETQQATLDIKADIWNMTRLRHVCTNASATLP
STKRPKSSKDNLVNRCLQTLSTIAPECCTAEVFTRTPNLKKLGVRGKIDALLESSKDGS
GSGLFSNIGKLGCLEYLKLVNDTRLSSKPLHLPPAYIFPQKLKKLSLVDTWFEWKDMSI
LGLLPELEVLKLKENAFKGQSWEQEDGGFPRLQVLWIERTDLTSWKASSGNFPRLKH
LALISCDKLEELPAELADVKNLQLIELQSSSESAARSARAILKRNQEKEQDGDKGTGFK
LSIFPHDLGL

1. A genetically altered plant, plant part thereof or plant cell, wherein the plant, part thereof or plant cell comprises at least one mutation in at least one nucleic acid sequence encoding a nucleotide-binding domain and leucine-rich repeat-containing (NLR) protein, wherein the NLR protein comprises a HD1 domain, and wherein the at least one mutation is a mutation of one or more amino acids in an N-terminal portion of the HD1 domain.

2-4. (canceled)

5. A genetically altered plant, plant part thereof or plant cell, wherein the plant, part thereof or plant cell expresses a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

6. The genetically altered plant, plant part thereof or plant cell of claim 1, wherein the NLR protein is a nucleotide-binding domain and leucine-rich repeat-containing required for cell-death (NRC) protein-, wherein optionally the NLR protein is NRC1 and/or NRC2 and/or NRC3.

7-8. (canceled)

9. The genetically altered plant, plant part thereof or plant cell of claim 1, wherein the N-terminal portion of the HD1 domain comprises no more than the N-terminal 42 amino acids of the HD1 domain, more preferably no more than the N-terminal 38 amino acids of the HD1 domain, optionally wherein the N-terminal portion of the HD1 domain comprises a sequence as defined in SEQ ID NO: 2, 5, 8, 14, 17, 23, 26 or 29 or a variant or fragment thereof.

10-11. (canceled)

12. The genetically altered plant, plant part thereof or plant cell of claim 1, wherein the at least one mutation is in a RNBS-C motif of the HD1 domain-, wherein optionally the RNBS-C motif comprises a sequence as defined in SEQ ID NO: 3, 6, 9, 15, 18, 24, 27 or 30 or variant or fragment thereof.

13-14. (canceled)

15. The genetically altered plant, plant part thereof or plant cell of claim 1, wherein the mutation is a substitution-, wherein optionally the substitution is at one or more positions in the NLR amino acid sequence, where the position is selected from 315, 316 or 317 (or a homologous position in a homologous sequence), of any of SEQ ID NO: 32, 34, 36, 41, 43, 47, 49 or 51 or a homolog or functional variant thereof, optionally wherein the substitution is a substitution to a hydrophilic amino acid and/or a positively charged amino acid; and optionally wherein the substitution is at position D317 or a homologous position in a homologous sequence, wherein preferably the substitution is D317K.

16-18. (canceled)

19. The genetically altered plant, plant part thereof or plant cell of claim 9, wherein the mutation is a substitution of all or a proportion of the N-terminal portion of the HD1 domain for a corresponding portion in a second NLR protein, wherein the second NLR protein is not inhibited by the pathogen effector.

20. The genetically altered plant, plant part thereof or plant cell of claim 12, wherein the mutation is a substitution of all or a significant proportion of the RNBS-C motif for a corresponding RNBS-C motif in a second NLR protein, wherein the second NLR protein is not inhibited by the pathogen effector.

21. The genetically altered plant, plant part thereof or plant cell of claim 5, wherein the nucleic acid construct expresses a nucleic acid sequence that encodes a NLR protein, wherein the NLR protein is selected from SEQ ID NO: 37, 38, 39, 44, 45, 52, 53, 54, 55, 56, 57, 58 or 59 and wherein preferably the regulatory sequence is operably linked to a regulatory sequence.

22-24. (canceled)

25. The genetically altered plant, plant part thereof or plant cell of claim 1, wherein the plant is a crop plant preferably a solanaceous plant.

26-27. (canceled)

28. A method of providing or improving pathogen resistance in a plant, the method comprising introducing at least one mutation into at least one nucleic acid sequence encoding a nucleotide-binding domain and leucine-rich repeat-containing (NLR) protein, wherein the NLR protein comprises a HD1 domain; and wherein the at least one mutation is a mutation of one or more amino acids in an N-terminal portion of the HD1 domain; or introducing and expressing a nucleic acid construct comprising a nucleic acid sequence encoding a mutated NLR protein, wherein the NLR protein comprises a HD1 domain, and wherein the NLR protein comprises a mutation of one or more amino acids in an N-terminal portion of the HD1 domain.

29-34. (canceled)

35. The method of claim 28, wherein the N-terminal portion of the HD1 domain comprises no more than the N-terminal 42 amino acids of the HD1 domain, more preferably no more than the N-terminal 38 amino acids of the HD1 domain, wherein preferably the N-terminal portion of the HD1 domain comprises a sequence as defined in SEQ ID NO: 2, 5, 8, 14, 17, 23, 26 or 29 or a variant or fragment thereof.

36-37. (canceled)

38. The method of claim 28, wherein the at least one mutation is in a RNBS-C motif of the HD1 domain-, wherein optionally the RNBS-C motif comprises a sequence as defined in SEQ ID NO: 3, 6, 9, 15, 18, 24, 27 or 30 or variant or fragment thereof.

39-40. (canceled)

41. The method of claim 28, wherein the mutation is a substitution-, wherein optionally wherein optionally the substitution is at one or more positions in the NLR amino acid sequence, where the position is selected from 315, 316 or 317 (or a homologous position in a homologous sequence), of any of SEQ ID NO: 32, 34, 36, 41, 43, 47, 49 or 51 or a homolog or functional variant thereof, optionally wherein the substitution is a substitution to a hydrophilic amino acid and/or a positively charged amino acid; and optionally wherein the substitution is at position D317 or a homologous position in a homologous sequence, wherein preferably the substitution is D317K.

42-43. (canceled)

44. The method of claim 35, wherein the mutation is a substitution of all or a significant proportion of the N-terminal portion of the HD1 domain for a corresponding portion of the HD1 domain in a second NLR protein, wherein the second NLR protein is not inhibited by the pathogen effector.

45. The method of claim 38, wherein the mutation is a substitution of all or a significant proportion of the RNBS-C motif for a corresponding RNBS-C motif in a second NLR protein, wherein the second NLR protein is not inhibited by the pathogen effector.

46. The method of claim 28, wherein the nucleic acid construct expresses a nucleic acid sequence that encodes a NLR protein, wherein the NLR protein is selected from SEQ ID NO: 37, 38, 39, 44, 45, 52, 53, 54, 55, 56, 57, 58 or 59 or a functional variant or homologue therein and wherein preferably the regulatory sequence is operably linked to a regulatory sequence.

47. The method of claim 28, wherein the pathogen is a potato cyst nematode.

48-50. (canceled)

51. The method of claim 28, wherein the NLR protein is NRC1 and/or NRC2 and/or NRC3.

52-55. (canceled)
